SEQUENCE LISTING (69 sequences)

SEQ ID NO: 1 - DNA, 1293 bp, Streptomyces coelicolor
  source 1..1293: mol_type = genomic DNA
ttgaccgcgcttgctgacaccacggaaccgacctacgacttcgtgggcatcgggctgggcccgttcaacctggggctggcctgcctgaccgagcccgtcgacgaactcgccggggtcttcctggagtccaagccggacttcgcgtggcacgcggggatgttcctcgacggcgcgcacctgcagacgccgttcatgtcggacctggtcaccctcgccgacccgacctccccctactccttcctcaactacctgaaggagaagggccggctgtactcgttctacatccgggagaacttctacccgctgcgcgtcgagtacgacgactactgccgctgggccgccgcacagctgagcagcatccgcttcggcaccaccgtcaccgaggtcacgtgggacgagacggccgaggtgtacgtggtggccacctccaccggtacgacctaccgcggccggcacctggtgctcggcaccggcaccgtgccgttcgtgcccgaggcgtgccgggggctggagggcgacctcttccacaacgcgcagtacatgcggcgcaaggcggagctgctggagaagaagtcggtcacggtcgtgggcagcgggcagtccgccgccgagatctactacgagctgctctccgggatcgacgcccacggctatcagctgaactgggtcacccgctccccgcggttcttcccgctggagtacaccaagctgaccctggagatgacgtccccggactacatcgactacttccgcgcgctgcccgaggacacccgctaccgcctggagaagcagcagaagggcctgttcaagggcatcaactcggacctgatcgacgcgatcttcgacctgctgtaccagaagaacgtggagagcggcggccgcggcgtgcccacccggctgctcaccaactcctcgctcaccaccgcccggtaccaggacggcacctacacgctcggcttccgccaggacgagcagggcaaggacttcgagatcgccaccgagggcctggtgctggccaccggctaccactacgagcagcccgccttcctggagccggtgcgtgaccggctgcgcttcgacggccacggccggttcgacgtcgcccgcaactacgcgatcgacgtcaccgggcgcggcgtgttcctgcagaacgcgggcgtgcacacgcacagcatcaccagccccgacctgggcatgggcgcctaccgcaacgcctcgatcatccgcgagctgctgggcaccgagtactacccggtcgagaagagcatcgccttccaggagttcgccgtatga

SEQ ID NO: 2 - DNA, 543 bp, Streptomyces coelicolor
  source 1..543: mol_type = genomic DNA
atgagcaccatcggtacgttcacgatccgcccgctcgacccggagaaggacgcggagctgctgcaccgctgggtgacgcatcccaaggcggcgttctggatgatgcaggacgcgaggctcgaggacgtcgagcgcgagtacatgcgcatcgccgcccacgagcaccaccacgcctacctgggcctgcacgacggcgagcccgccttcctgatggagaagtacgacccccggcacgtcgaactcgtcgggctctacgagcccgagcccggtgacgtcggcatgcacttcctggtcgcccccaccgaccgccccgtccacggcttcacccgcgccgtcatcaccgccgtgatggaggagctgttcgccgacccggccacccgccgggtcgtggtggagccggacgtcggcaacaaggccgtgcacgccctgaacgaggccgtgggttttgaacccgagcgcgagatcgacaagccggagaagcgcgcgctgctgagcttctgcacgcgtgaacagttcgaggccgcccgaggagcttccgcatga

SEQ ID NO: 3 - DNA, 1773 bp, Streptomyces coelicolor
  source 1..1773: mol_type = genomic DNA
atgaccctgtccgacgccgtggcgcacctgtccccccaccgctgggcgcgggccaaccgtctgctgatccgcaaggcgctcgccgagttcgcgcacgagcggctcgtcacgccggagccggccggcgacggccggtacgtcgtccgcagcgacgacggcgagacccgctacacgttcaccgcgaagctgcgcggcctggaccactggcagatcgacgccggctcgatcacccgtcatcgcgacggcgtcgaactcgagctcgccgcgctggacttcttcatcgagctgcagaagtccctggggctgagcgacgagatcctgccggtgtacctggaggagatctcctccaccctctccggcacctgctacaagctgaccaagccggaggtcacctcggccgagctggtcgacgccgggttccaggcgatcgagaccggcatgaccgagggccacccctgcttcgtcgccaacaacgggcggctcggcttcggcgtccacgagtaccggtggtacgccccggaggccgcgaacccggtgcggctggtgtggctggccgcccaccactcgcgggccgcgttcaccgcgggtgcgggcatcgagtacgagtcgttcgtccgccaggagctgggcgaggagaccgtcgaccggttccacgacgtcctgcgcgcgcaggacctggaccccgccgactacctgctggtccccgtccacccctggcagtggtggaacaagctgtccgtcaccttcgccgccgaggtcgcccgccggcacctggtgttgctgggcgagggcgacgacgagtacctggcgcagcagtccatacgtaccttcttcaaccgctcccatccggagaagcactatgtgaagacggcgctgtccgtcctcaacatgggcttcatgaggggcctgtcggccgcctacatggaggccactccggccatcaacgactggctggcccgcctgatcgacaacgacccggtgctgcggtccacgggactgtcgatcatccgggagcgcgccgccgtggggtaccggcacctggagtacgagcaggccaccgaccgctactcgccgtaccgcaagatgctggcggcgctgtggcgggagagcccggtggcgtccctggaggacggcgagtcgctcgccaccatggcgtccctggtccacgtcgaccacgagggccggtccttcgccgccgccctgatcgagcgctccgggctcaccccggccgagtggctgcggcgctacctgcgggcctactacgtcccgctgctgcacagcttctacgcctacgacctggtgtacatgccccacggcgagaacgtcatcctggtgctggaggacggcgccgtgaagcgcgcggtctacaaggacatcgccgaggagatcgcggtgatggacccggacgcggtactgccgccggaggtgtcccggatccgcgtcgaggtccccgacgacaccaagctgctgtccatcttcacggacgtcttcgactgcttcttccgcttcctcgcggcgaacctcgccgaggagggcgttctcgacgaggacggcttctggcggacggtcgcggaggtcacccgcgagtaccaggcgtcggtgccggagctgacggacaagttcgcccggtacgacctgttcgccccggagttcgcgctgtcctgcctgaaccgcctccagttgcgcaacaacaagcagatggtcgacctggcggacccctcgggcgccctccagctcgtgggcaccctgaggaaccccctggcggccttctga

SEQ ID NO: 4 - protein, 430 aa, Streptomyces coelicolor
LTALADTTEPTYDFVGIGLGPFNLGLACLTEPVDELAGVFLESKPDFAWHAGMFLDGAHLQTPFMSDLVTLADPTSPYSFLNYLKEKGRLYSFYIRENFYPLRVEYDDYCRWAAAQLSSIRFGTTVTEVTWDETAEVYVVATSTGTTYRGRHLVLGTGTVPFVPEACRGLEGDLFHNAQYMRRKAELLEKKSVTVVGSGQSAAEIYYELLSGIDAHGYQLNWVTRSPRFFPLEYTKLTLEMTSPDYIDYFRALPEDTRYRLEKQQKGLFKGINSDLIDAIFDLLYQKNVESGGRGVPTRLLTNSSLTTARYQDGTYTLGFRQDEQGKDFEIATEGLVLATGYHYEQPAFLEPVRDRLRFDGHGRFDVARNYAIDVTGRGVFLQNAGVHTHSITSPDLGMGAYRNASIIRELLGTEYYPVEKSIAFQEFAV

SEQ ID NO: 5 - protein, 180 aa, Streptomyces coelicolor
MSTIGTFTIRPLDPEKDAELLHRWVTHPKAAFWMMQDARLEDVEREYMRIAAHEHHHAYLGLHDGEPAFLMEKYDPRHVELVGLYEPEPGDVGMHFLVAPTDRPVHGFTRAVITAVMEELFADPATRRVVVEPDVGNKAVHALNEAVGFEPEREIDKPEKRALLSFCTREQFEAARGASA

SEQ ID NO: 6 - protein, 590 aa, Streptomyces coelicolor
MTLSDAVAHLSPHRWARANRLLIRKALAEFAHERLVTPEPAGDGRYVVRSDDGETRYTFTAKLRGLDHWQIDAGSITRHRDGVELELAALDFFIELQKSLGLSDEILPVYLEEISSTLSGTCYKLTKPEVTSAELVDAGFQAIETGMTEGHPCFVANNGRLGFGVHEYRWYAPEAANPVRLVWLAAHHSRAAFTAGAGIEYESFVRQELGEETVDRFHDVLRAQDLDPADYLLVPVHPWQWWNKLSVTFAAEVARRHLVLLGEGDDEYLAQQSIRTFFNRSHPEKHYVKTALSVLNMGFMRGLSAAYMEATPAINDWLARLIDNDPVLRSTGLSIIRERAAVGYRHLEYEQATDRYSPYRKMLAALWRESPVASLEDGESLATMASLVHVDHEGRSFAAALIERSGLTPAEWLRRYLRAYYVPLLHSFYAYDLVYMPHGENVILVLEDGAVKRAVYKDIAEEIAVMDPDAVLPPEVSRIRVEVPDDTKLLSIFTDVFDCFFRFLAANLAEEGVLDEDGFWRTVAEVTREYQASVPELTDKFARYDLFAPEFALSCLNRLQLRNNKQMVDLADPSGALQLVGTLRNPLAAF

SEQ ID NO: 7 - DNA, 3732 bp, synthetic construct
  misc_feature 1..3732: note = vector construct
  source 1..3732: mol_type = other DNA
aagcttccgtcctcgacctgatcgccggccacgccgagcagtacctgggagagtcccttgaccgcgcttgctgacaccacggaaccgacctacgacttcgtgggcatcgggctgggcccgttcaacctggggctggcctgcctgaccgagcccgtcgacgaactcgccggggtcttcctggagtccaagccggacttcgcgtggcacgcggggatgttcctcgacggcgcgcacctgcagacgccgttcatgtcggacctggtcaccctcgccgacccgacctccccctactccttcctcaactacctgaaggagaagggccggctgtactcgttctacatccgggagaacttctacccgctgcgcgtcgagtacgacgactactgccgctgggccgccgcacagctgagcagcatccgcttcggcaccaccgtcaccgaggtcacgtgggacgagacggccgaggtgtacgtggtggccacctccaccggtacgacctaccgcggccggcacctggtgctcggcaccggcaccgtgccgttcgtgcccgaggcgtgccgggggctggagggcgacctcttccacaacgcgcagtacatgcggcgcaaggcggagctgctggagaagaagtcggtcacggtcgtgggcagcgggcagtccgccgccgagatctactacgagctgctctccgggatcgacgcccacggctatcagctgaactgggtcacccgctccccgcggttcttcccgctggagtacaccaagctgaccctggagatgacgtccccggactacatcgactacttccgcgcgctgcccgaggacacccgctaccgcctggagaagcagcagaagggcctgttcaagggcatcaactcggacctgatcgacgcgatcttcgacctgctgtaccagaagaacgtggagagcggcggccgcggcgtgcccacccggctgctcaccaactcctcgctcaccaccgcccggtaccaggacggcacctacacgctcggcttccgccaggacgagcagggcaaggacttcgagatcgccaccgagggcctggtgctggccaccggctaccactacgagcagcccgccttcctggagccggtgcgtgaccggctgcgcttcgacggccacggccggttcgacgtcgcccgcaactacgcgatcgacgtcaccgggcgcggcgtgttcctgcagaacgcgggcgtgcacacgcacagcatcaccagccccgacctgggcatgggcgcctaccgcaacgcctcgatcatccgcgagctgctgggcaccgagtactacccggtcgagaagagcatcgccttccaggagttcgccgtatgagcaccatcggtacgttcacgatccgcccgctcgacccggagaaggacgcggagctgctgcaccgctgggtgacgcatcccaaggcggcgttctggatgatgcaggacgcgaggctcgaggacgtcgagcgcgagtacatgcgcatcgccgcccacgagcaccaccacgcctacctgggcctgcacgacggcgagcccgccttcctgatggagaagtacgacccccggcacgtcgaactcgtcgggctctacgagcccgagcccggtgacgtcggcatgcacttcctggtcgcccccaccgaccgccccgtccacggcttcacccgcgccgtcatcaccgccgtgatggaggagctgttcgccgacccggccacccgccgggtcgtggtggagccggacgtcggcaacaaggccgtgcacgccctgaacgaggccgtgggttttgaacccgagcgcgagatcgacaagccggagaagcgcgcgctgctgagcttctgcacgcgtgaacagttcgaggccgcccgaggagcttccgcatgaccctgtccgacgccgtggcgcacctgtccccccaccgctgggcgcgggccaaccgtctgctgatccgcaaggcgctcgccgagttcgcgcacgagcggctcgtcacgccggagccggccggcgacggccggtacgtcgtccgcagcgacgacggcgagacccgctacacgttcaccgcgaagctgcgcggcctggaccactggcagatcgacgccggctcgatcacccgtcatcgcgacggcgtcgaactcgagctcgccgcgctggacttcttcatcgagctgcagaagtccctggggctgagcgacgagatcctgccggtgtacctggaggagatctcctccaccctctccggcacctgctacaagctgaccaagccggaggtcacctcggccgagctggtcgacgccgggttccaggcgatcgagaccggcatgaccgagggccacccctgcttcgtcgccaacaacgggcggctcggcttcggcgtccacgagtaccggtggtacgccccggaggccgcgaacccggtgcggctggtgtggctggccgcccaccactcgcgggccgcgttcaccgcgggtgcgggcatcgagtacgagtcgttcgtccgccaggagctgggcgaggagaccgtcgaccggttccacgacgtcctgcgcgcgcaggacctggaccccgccgactacctgctggtccccgtccacccctggcagtggtggaacaagctgtccgtcaccttcgccgccgaggtcgcccgccggcacctggtgttgctgggcgagggcgacgacgagtacctggcgcagcagtccatacgtaccttcttcaaccgctcccatccggagaagcactatgtgaagacggcgctgtccgtcctcaacatgggcttcatgaggggcctgtcggccgcctacatggaggccactccggccatcaacgactggctggcccgcctgatcgacaacgacccggtgctgcggtccacgggactgtcgatcatccgggagcgcgccgccgtggggtaccggcacctggagtacgagcaggccaccgaccgctactcgccgtaccgcaagatgctggcggcgctgtggcgggagagcccggtggcgtccctggaggacggcgagtcgctcgccaccatggcgtccctggtccacgtcgaccacgagggccggtccttcgccgccgccctgatcgagcgctccgggctcaccccggccgagtggctgcggcgctacctgcgggcctactacgtcccgctgctgcacagcttctacgcctacgacctggtgtacatgccccacggcgagaacgtcatcctggtgctggaggacggcgccgtgaagcgcgcggtctacaaggacatcgccgaggagatcgcggtgatggacccggacgcggtactgccgccggaggtgtcccggatccgcgtcgaggtccccgacgacaccaagctgctgtccatcttcacggacgtcttcgactgcttcttccgcttcctcgcggcgaacctcgccgaggagggcgttctcgacgaggacggcttctggcggacggtcgcggaggtcacccgcgagtaccaggcgtcggtgccggagctgacggacaagttcgcccggtacgacctgttcgccccggagttcgcgctgtcctgcctgaaccgcctccagttgcgcaacaacaagcagatggtcgacctggcggacccctcgggcgccctccagctcgtgggcaccctgaggaaccccctggcggccttctgaccgcatcggacggacgagcccccggagtacggtcctccgggggcttcgtcacaccctcaggcgcgacgaagctt

SEQ ID NO: 8 - DNA, 10317 bp, synthetic construct
  misc_feature 1..10317: note = Vector construct
  source 1..10317: mol_type = other DNA
aattaccgtcctcgacctgatcgccggccacgccgagcagtacctgggagagtcccttgaccgcgcttgctgacaccacggaaccgacctacgacttcgtgggcatcgggctgggcccgttcaacctggggctggcctgcctgaccgagcccgtcgacgaactcgccggggtcttcctggagtccaagccggacttcgcgtggcacgcggggatgttcctcgacggcgcgcacctgcagacgccgttcatgtcggacctggtcaccctcgccgacccgacctccccctactccttcctcaactacctgaaggagaagggccggctgtactcgttctacatccgggagaacttctacccgctgcgcgtcgagtacgacgactactgccgctgggccgccgcacagctgagcagcatccgcttcggcaccaccgtcaccgaggtcacgtgggacgagacggccgaggtgtacgtggtggccacctccaccggtacgacctaccgcggccggcacctggtgctcggcaccggcaccgtgccgttcgtgcccgaggcgtgccgggggctggagggcgacctcttccacaacgcgcagtacatgcggcgcaaggcggagctgctggagaagaagtcggtcacggtcgtgggcagcgggcagtccgccgccgagatctactacgagctgctctccgggatcgacgcccacggctatcagctgaactgggtcacccgctccccgcggttcttcccgctggagtacaccaagctgaccctggagatgacgtccccggactacatcgactacttccgcgcgctgcccgaggacacccgctaccgcctggagaagcagcagaagggcctgttcaagggcatcaactcggacctgatcgacgcgatcttcgacctgctgtaccagaagaacgtggagagcggcggccgcggcgtgcccacccggctgctcaccaactcctcgctcaccaccgcccggtaccaggacggcacctacacgctcggcttccgccaggacgagcagggcaaggacttcgagatcgccaccgagggcctggtgctggccaccggctaccactacgagcagcccgccttcctggagccggtgcgtgaccggctgcgcttcgacggccacggccggttcgacgtcgcccgcaactacgcgatcgacgtcaccgggcgcggcgtgttcctgcagaacgcgggcgtgcacacgcacagcatcaccagccccgacctgggcatgggcgcctaccgcaacgcctcgatcatccgcgagctgctgggcaccgagtactacccggtcgagaagagcatcgccttccaggagttcgccgtatgagcaccatcggtacgttcacgatccgcccgctcgacccggagaaggacgcggagctgctgcaccgctgggtgacgcatcccaaggcggcgttctggatgatgcaggacgcgaggctcgaggacgtcgagcgcgagtacatgcgcatcgccgcccacgagcaccaccacgcctacctgggcctgcacgacggcgagcccgccttcctgatggagaagtacgacccccggcacgtcgaactcgtcgggctctacgagcccgagcccggtgacgtcggcatgcacttcctggtcgcccccaccgaccgccccgtccacggcttcacccgcgccgtcatcaccgccgtgatggaggagctgttcgccgacccggccacccgccgggtcgtggtggagccggacgtcggcaacaaggccgtgcacgccctgaacgaggccgtgggttttgaacccgagcgcgagatcgacaagccggagaagcgcgcgctgctgagcttctgcacgcgtgaacagttcgaggccgcccgaggagcttccgcatgaccctgtccgacgccgtggcgcacctgtccccccaccgctgggcgcgggccaaccgtctgctgatccgcaaggcgctcgccgagttcgcgcacgagcggctcgtcacgccggagccggccggcgacggccggtacgtcgtccgcagcgacgacggcgagacccgctacacgttcaccgcgaagctgcgcggcctggaccactggcagatcgacgccggctcgatcacccgtcatcgcgacggcgtcgaactcgagctcgccgcgctggacttcttcatcgagctgcagaagtccctggggctgagcgacgagatcctgccggtgtacctggaggagatctcctccaccctctccggcacctgctacaagctgaccaagccggaggtcacctcggccgagctggtcgacgccgggttccaggcgatcgagaccggcatgaccgagggccacccctgcttcgtcgccaacaacgggcggctcggcttcggcgtccacgagtaccggtggtacgccccggaggccgcgaacccggtgcggctggtgtggctggccgcccaccactcgcgggccgcgttcaccgcgggtgcgggcatcgagtacgagtcgttcgtccgccaggagctgggcgaggagaccgtcgaccggttccacgacgtcctgcgcgcgcaggacctggaccccgccgactacctgctggtccccgtccacccctggcagtggtggaacaagctgtccgtcaccttcgccgccgaggtcgcccgccggcacctggtgttgctgggcgagggcgacgacgagtacctggcgcagcagtccatacgtaccttcttcaaccgctcccatccggagaagcactatgtgaagacggcgctgtccgtcctcaacatgggcttcatgaggggcctgtcggccgcctacatggaggccactccggccatcaacgactggctggcccgcctgatcgacaacgacccggtgctgcggtccacgggactgtcgatcatccgggagcgcgccgccgtggggtaccggcacctggagtacgagcaggccaccgaccgctactcgccgtaccgcaagatgctggcggcgctgtggcgggagagcccggtggcgtccctggaggacggcgagtcgctcgccaccatggcgtccctggtccacgtcgaccacgagggccggtccttcgccgccgccctgatcgagcgctccgggctcaccccggccgagtggctgcggcgctacctgcgggcctactacgtcccgctgctgcacagcttctacgcctacgacctggtgtacatgccccacggcgagaacgtcatcctggtgctggaggacggcgccgtgaagcgcgcggtctacaaggacatcgccgaggagatcgcggtgatggacccggacgcggtactgccgccggaggtgtcccggatccgcgtcgaggtccccgacgacaccaagctgctgtccatcttcacggacgtcttcgactgcttcttccgcttcctcgcggcgaacctcgccgaggagggcgttctcgacgaggacggcttctggcggacggtcgcggaggtcacccgcgagtaccaggcgtcggtgccggagctgacggacaagttcgcccggtacgacctgttcgccccggagttcgcgctgtcctgcctgaaccgcctccagttgcgcaacaacaagcagatggtcgacctggcggacccctcgggcgccctccagctcgtgggcaccctgaggaaccccctggcggccttctgaccgcatcggacggacgagcccccggagtacggtcctccgggggcttcgtcacaccctcaggcgcgacgtgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 9 - DNA, 3779 bp, synthetic construct
  misc_feature 1..3779: note = Vector construct
  source 1..3779: mol_type = other DNA
aagcttccgtcctcgacctgatcgccggccacgccgtgcaggaaggagatatagatatgaccgctcttgctgacaccaccgagccaacctacgacttcgtcggcatcggtcttggacctttcaacctgggtctggcatgtctcaccgagccagtcgacgagctagcaggtgttttcctggaatccaagccagacttcgcttggcacgcgggaatgttcctggacggcgcccacctgcagaccccattcatgtccgacctggtcaccctggctgaccctaccagcccttacagctttctgaactaccttaaggagaagggacgtctttacagcttctacatccgcgaaaacttttacccactgcgtgtggaatacgacgattactgccgttgggcagctgcacagttatcctccatccgcttcggtaccacagtcaccgaagtcacctgggatgaaaccgctgaagtgtacgtcgtcgcgaccagcaccggtaccacgtaccgcggtcgtcatctggttctgggaaccggcaccgtcccgttcgtccctgaggcatgccgcggattggaaggcgaccttttccacaacgcacagtacatgcgtcgcaaggcagaactcctcgagaagaagtcggttaccgttgttggctccggccagtccgctgctgaaatctactacgagctcctttccggtatcgacgcacacggctaccaactaaactgggtcacccgctccccccgcttcttccctctcgaatacaccaagctcaccctcgaaatgacctcccccgactacattgattactttcgcgctctgcctgaagacacccgctaccgcctggaaaagcaacagaagggcctgttcaagggcatcaattcagacctcatcgacgcaatcttcgacctcctgtaccagaagaacgtcgaatccggtggccgcggcgtccccactcgcctgctcaccaactcctccctgaccaccgcccgctaccaggacggcacctacacacttggcttccgccaagacgaacagggaaaggatttcgaaatcgctactgagggcctggtcctcgccaccggctaccactacgaacagccagccttcctagagccagtccgcgatcgcctccgcttcgacggtcacggccgttttgatgtagcacgcaactacgctatcgatgttacaggccgcggcgtgttcctccagaacgcaggcgttcacacccactccatcacctccccagacctcggcatgggcgcataccgcaatgccagcattatccgcgaactgctgggcaccgaatactacccagtcgagaaatccatcgcattccaagaattcgccgtctgatgcaggaaggagatatagatatgtccaccatcggtaccttcaccatccgccctcttgacccagagaaggacgcggagttgctgcaccgctgggtgacccacccaaaggccgcattctggatgatgcaggacgcacgcctagaagacgtagagcgcgaatacatgcgcatcgctgctcacgagcaccaccacgcctacctgggcctgcacgacggagagcctgctttcctcatggaaaaatacgacccacgccacgtcgaactggtgggcctgtacgaaccagaaccaggagacgttggcatgcacttcctcgtagcgccaaccgaccgcccagttcacggcttcacccgcgccgtcatcaccgccgttatggaagaactgttcgctgatcctgcgacccgccgcgtcgtcgttgagccagacgtcggcaacaaggctgtgcacgccctcaacgaagcagttggcttcgaacccgaacgcgaaatcgacaagccagagaagcgcgcactgctttccttctgcacccgcgaacagttcgaagcagctcgcggcgcctccgcctgatgcaggaaggagatatagatatgaccctgtccgacgccgtggctcacctgtccccacaccgctgggctcgcgcaaaccgtctgctcatccgcaaagctctggcagagttcgctcacgaacgtctcgttacccctgaacccgcaggcgacggtcgctacgttgttcgctccgacgacggcgagacccgctacactttcaccgctaagctccgcggcctagaccactggcagatcgacgcaggctccattacccgccaccgcgatggcgttgagctcgagctggctgcactggacttcttcatcgaactccagaaatccctcgggctctctgacgagatcctgccagtctatctggaggagatctcctcgaccctttctggtacctgctacaagctgaccaaacccgaagtcacctccgcagagctggtcgacgcaggcttccaggctatcgaaactggtatgaccgaaggtcacccctgcttcgttgcaaacaatggccgtctgggcttcggcgttcacgaataccgctggtacgctccagaggccgcaaacccagttcgtctagtttggctcgcagcccaccactcccgcgcagctttcaccgcaggcgctggaatcgagtacgaatccttcgttcgccaggagcttggcgaagagaccgtcgatcgtttccacgacgttctgcgcgcccaagacctcgaccctgctgattacctgctcgtcccagttcatccatggcaatggtggaacaagctgagcgtcacctttgcagcggaggtcgcccgtcgccacctcgtcctgctgggcgaaggtgatgacgaataccttgcacagcaaagcattcgcacctttttcaaccgttcccaccctgaaaagcactacgtcaagactgccctctcagtactcaatatgggcttcatgcgcggcctctccgcagcatacatggaggccaccccagcaatcaacgactggcttgctcgccttatcgataatgacccagttctccgttccaccggcctgtccattatccgcgaacgtgctgcagttggctaccggcaccttgaatacgaacaggcaaccgaccgctactccccataccgcaaaatgttggcagcactttggcgcgaatccccagtagcatccctcgaggacggcgagtccctggccaccatggcttcactcgttcacgtggaccacgaaggccgctccttcgctgctgctctgatcgagcgctctggtctcaccccagctgaatggctgcgccgctaccttcgtgcttactacgtgccattgctgcactcattctacgcatacgatctcgtctacatgccacacggcgagaacgtcatcttggtgctcgaggatggcgcggtgaagcgcgcagtctacaaggacatcgctgaagagatcgcagtgatggacccagacgcagttctgccacctgaggtttcccgcatccgcgtcgaagttccagacgacaccaagctcctgagcatcttcaccgatgtcttcgactgcttcttccgtttcctggctgctaatctcgcggaagaaggcgtcctcgatgaggacggtttctggcgtaccgtcgcagaggtgacacgcgaataccaagcatcagttccagagctcaccgacaagttcgcacgttacgatcttttcgcacccgaattcgctctgtcctgcctgaaccgcctccagctccgcaacaacaagcagatggttgatctcgcagacccttccggcgccctgcagctggttggaaccctccgcaaccctctcgcggcattctgaccgcatcggacggacgagcccccggagtacggtcctccgggggcttcgtcacaccctcaggcgcgacgaagctt

SEQ ID NO: 10 - DNA, 10414 bp, synthetic construct
  misc_feature 1..10414: note = vector construct
  source 1..10414: mol_type = other DNA
aattattcacacaggaaacagaattaattaccgtcctcgacctgatcgccggccacgccgtgcaggaaggagatatagatatgaccgctcttgctgacaccaccgagccaacctacgacttcgtcggcatcggtcttggacctttcaacctgggtctggcatgtctcaccgagccagtcgacgagctagcaggtgttttcctggaatccaagccagacttcgcttggcacgcgggaatgttcctggacggcgcccacctgcagaccccattcatgtccgacctggtcaccctggctgaccctaccagcccttacagctttctgaactaccttaaggagaagggacgtctttacagcttctacatccgcgaaaacttttacccactgcgtgtggaatacgacgattactgccgttgggcagctgcacagttatcctccatccgcttcggtaccacagtcaccgaagtcacctgggatgaaaccgctgaagtgtacgtcgtcgcgaccagcaccggtaccacgtaccgcggtcgtcatctggttctgggaaccggcaccgtcccgttcgtccctgaggcatgccgcggattggaaggcgaccttttccacaacgcacagtacatgcgtcgcaaggcagaactcctcgagaagaagtcggttaccgttgttggctccggccagtccgctgctgaaatctactacgagctcctttccggtatcgacgcacacggctaccaactaaactgggtcacccgctccccccgcttcttccctctcgaatacaccaagctcaccctcgaaatgacctcccccgactacattgattactttcgcgctctgcctgaagacacccgctaccgcctggaaaagcaacagaagggcctgttcaagggcatcaattcagacctcatcgacgcaatcttcgacctcctgtaccagaagaacgtcgaatccggtggccgcggcgtccccactcgcctgctcaccaactcctccctgaccaccgcccgctaccaggacggcacctacacacttggcttccgccaagacgaacagggaaaggatttcgaaatcgctactgagggcctggtcctcgccaccggctaccactacgaacagccagccttcctagagccagtccgcgatcgcctccgcttcgacggtcacggccgttttgatgtagcacgcaactacgctatcgatgttacaggccgcggcgtgttcctccagaacgcaggcgttcacacccactccatcacctccccagacctcggcatgggcgcataccgcaatgccagcattatccgcgaactgctgggcaccgaatactacccagtcgagaaatccatcgcattccaagaattcgccgtctgatgcaggaaggagatatagatatgtccaccatcggtaccttcaccatccgccctcttgacccagagaaggacgcggagttgctgcaccgctgggtgacccacccaaaggccgcattctggatgatgcaggacgcacgcctagaagacgtagagcgcgaatacatgcgcatcgctgctcacgagcaccaccacgcctacctgggcctgcacgacggagagcctgctttcctcatggaaaaatacgacccacgccacgtcgaactggtgggcctgtacgaaccagaaccaggagacgttggcatgcacttcctcgtagcgccaaccgaccgcccagttcacggcttcacccgcgccgtcatcaccgccgttatggaagaactgttcgctgatcctgcgacccgccgcgtcgtcgttgagccagacgtcggcaacaaggctgtgcacgccctcaacgaagcagttggcttcgaacccgaacgcgaaatcgacaagccagagaagcgcgcactgctttccttctgcacccgcgaacagttcgaagcagctcgcggcgcctccgcctgatgcaggaaggagatatagatatgaccctgtccgacgccgtggctcacctgtccccacaccgctgggctcgcgcaaaccgtctgctcatccgcaaagctctggcagagttcgctcacgaacgtctcgttacccctgaacccgcaggcgacggtcgctacgttgttcgctccgacgacggcgagacccgctacactttcaccgctaagctccgcggcctagaccactggcagatcgacgcaggctccattacccgccaccgcgatggcgttgagctcgagctggctgcactggacttcttcatcgaactccagaaatccctcgggctctctgacgagatcctgccagtctatctggaggagatctcctcgaccctttctggtacctgctacaagctgaccaaacccgaagtcacctccgcagagctggtcgacgcaggcttccaggctatcgaaactggtatgaccgaaggtcacccctgcttcgttgcaaacaatggccgtctgggcttcggcgttcacgaataccgctggtacgctccagaggccgcaaacccagttcgtctagtttggctcgcagcccaccactcccgcgcagctttcaccgcaggcgctggaatcgagtacgaatccttcgttcgccaggagcttggcgaagagaccgtcgatcgtttccacgacgttctgcgcgcccaagacctcgaccctgctgattacctgctcgtcccagttcatccatggcaatggtggaacaagctgagcgtcacctttgcagcggaggtcgcccgtcgccacctcgtcctgctgggcgaaggtgatgacgaataccttgcacagcaaagcattcgcacctttttcaaccgttcccaccctgaaaagcactacgtcaagactgccctctcagtactcaatatgggcttcatgcgcggcctctccgcagcatacatggaggccaccccagcaatcaacgactggcttgctcgccttatcgataatgacccagttctccgttccaccggcctgtccattatccgcgaacgtgctgcagttggctaccggcaccttgaatacgaacaggcaaccgaccgctactccccataccgcaaaatgttggcagcactttggcgcgaatccccagtagcatccctcgaggacggcgagtccctggccaccatggcttcactcgttcacgtggaccacgaaggccgctccttcgctgctgctctgatcgagcgctctggtctcaccccagctgaatggctgcgccgctaccttcgtgcttactacgtgccattgctgcactcattctacgcatacgatctcgtctacatgccacacggcgagaacgtcatcttggtgctcgaggatggcgcggtgaagcgcgcagtctacaaggacatcgctgaagagatcgcagtgatggacccagacgcagttctgccacctgaggtttcccgcatccgcgtcgaagttccagacgacaccaagctcctgagcatcttcaccgatgtcttcgactgcttcttccgtttcctggctgctaatctcgcggaagaaggcgtcctcgatgaggacggtttctggcgtaccgtcgcagaggtgacacgcgaataccaagcatcagttccagagctcaccgacaagttcgcacgttacgatcttttcgcacccgaattcgctctgtcctgcctgaaccgcctccagctccgcaacaacaagcagatggttgatctcgcagacccttccggcgccctgcagctggttggaaccctccgcaaccctctcgcggcattctgaccgcatcggacggacgagcccccggagtacggtcctccgggggcttcgtcacaccctcaggcgcgacgtgcatgcctgcaggtcgactctagatgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 11 - DNA, 1443 bp, Streptomyces violaceoruber
  source 1..1443: mol_type = genomic DNA
atgcgctcgcacctgctcaacgacaccaccgcggagcagtaccgccgttccgtgaccgaaggagtcgagcgggtggccgccaaactcgccaccaccgaccgcccgttcaccggcgtcacggtcgacgccctctccccccgcatcgacgcgatcgacctcgacgagccgctgcacgacaccgccgcggtcctcgacgagctggaggacgtctacctccgcgacgccgtctacttccaccacccccgctacctcgcccacctcaactgcccggtcgtcataccggcgctgctcggtgaagcggtcctgtccgccgtcaactcctccctggacacctgggaccagtcggccggcggcacgctcatcgagcggaagctgatcgactggacctgcgcccgcatcggcctcggcccggcggccgacggcgtgttcacctccggcggcacccagtccaacctccaggcgctgctcctcgcccgcgaggaggcgaaggccgaggacttcgccgacctgcggatcttcgcctccgaggccagccacttcagcgtcaggaagtccgcgaaactgctcggcctcggccccgacgccgtcgtgtcgatcccggtcgaccgcgacaagcggatgcagaccgtcgccctcgcccgcgagctggagcgctgcgcgcgggacggcctggtccccatggccgtcgtcgccaccggcggcaccaccgacttcggctcgatcgacccgctgccggagatcgccgggctgtgcgagcagtacggcgtgtggatgcacgtcgacgcggcctacggctgcgggctgctcgcctccctgaagtaccgggaccgcatcaccggcatcgagcgggccgactcggtcaccgtggactaccacaagtccttcttccagccggtgagttcgtcggccgtgctggtccgggacgcggccaccctgcgccacgccacctaccacgcggagtacctcaacccgcgccgcatggtgcaggaacgtatccccaaccaggtggacaagtccctccagaccacccgccgcttcgacgcgctcaagctgtggatgacgctgcgcgtgatgggcgccgacggcatcggcgtcctcttcgacgaggtgtgcgacctggccgccgagggctggaaactgctcgccgccgacccgcgcttcgacgtcgtggtccagccgtcgctgtccacgctggtcttccgccacatcccggcggacgtcaccgatcccgccgagatcgaccgcgccaacctgtacgcccgcaaggccctgttcgcgtccggcgacgccgtggtcgcgggcaccaaggtcgccggtcgccactacctgaagttcaccctgctcaaccccgagacgaccccggccgacatcgccgccgtcctcgacctgatcgccggccacgccgagcagtacctgggagactcccttgaccgcgcttcctga

SEQ ID NO: 12 - DNA, 1281 bp, Streptomyces violaceoruber
  source 1..1281: mol_type = genomic DNA
ttgaccgcgcttcctgaagccagcgccccgtacgacttcgtgggcatcggcctcggccccttcaacctcggcctcgcctgcctcaccgagcccgtcgccgagctgaacggcgtcttcctggagtccaagccggacttcgagtggcacgccgggatgttcctggacggcgcccacctccagaccccgttcatgtcggacctggtgacgctcgccgacccgacctcgccgtactccttcctcaactacctgaaggagcagggccggctgtactccttctacatccgggagaacttctacccgctgcgggtcgagtacgacgactactgccgctgggccgcgcgcaagctgagcagcgtccgcttctccaccacggtcaccgaggtcacgtacgacgagcgcgaggagctgtacgccgtcgccaccacgtccggcgacacctaccgcgcccgccgcctcgtcctcggcaccggcaccccgccgcacatcccggacgcctgccggggcctggccggcgacttcctccacaactcccggtacgtgcggcaccgggcggagctggtgaagaagaagtcgatcacgctggtcggcagcggccagtccgccgccgagatctaccaggacctgctgagcgagatcgacgtccacggctacgggctgaactgggtgacccgctccccgcggttcttcccgctcgaatacaccaagctcaccctggagatgacgtccccggagtacgtggactactaccacgcgctgcccgaggacacccgctaccgcctcacggccgagcagaagggcctgttcaagggcatcgacggcgacctgatcaacgagatcttcgacctgctctaccagaaacggctcggcggccccgtccccacccgcctgctcaccaactcggcgctgaccagcgcccggtatgcggacggcacctacacgctcgggttccgccaggaggagcagggcacggacttcgagatcgagaccgagggcctggtcctcgccaccggctaccggtacaccgagccggagttcctcaagcccgtccgggaccggctgcgctacgactcccgcggcaacttcgacatcggccgcaactacgccgtcgacgtcacgggaggcggcgtgttcctccagaacgcgggggtccacgcgcacagcgtcaccagccccgacctgggcatgggcgcctaccgcaacagctgcatcgtccgggagctgctcggacgcgagtactacccggtcgagcagtcgatcgcgttccaggagttcgccgtatga

SEQ ID NO: 13 - DNA, 555 bp, Streptomyces violaceoruber
  source 1..555: mol_type = genomic DNA
atgagccgcttgagcaccaccacccccgtcggggcactgaccctgcgccccgtcgacccgctgacggacgccgtactgctgcacggctggctcacccaccccaagtccgcgttctggatgatgcaggacgcccggctggtggacgtcgagcgggcctacatggagctggccgccgacgagcaccagcaggcccacctcggcctgcacgacggggtcccggccttcctgacggagcgctacgaccccgcccaccgcgaactggtcgggctgtacgagcccgagccgggcgacgtcggcatgcacttcctggtcgcgcccaccgaccggcccgtgcacggcttcacccgcgccgtgatcaccaccgtgatgacggagctgttcgccgacccggcgacccggcgggtcgtcgtcgaaccggacgtcaccaacaccgccgtgcacgccctgaacgcagccgtcggattcgtgcccgagcgcgagatccagaagccggagaagaaggccttgctgagcttctgcacccgcgagcagttcgcgaaggcggtgtccgcatga

SEQ ID NO: 14 - DNA, 1788 bp, Streptomyces violaceoruber
  source 1..1788: mol_type = genomic DNA
atgagcctcgccgacgccgtcgcccacctgacccccgaacgctgggaggaggccaaccgcctcctggtccgcaaggcgctggccgagttcacccacgagcggctgctgacccccgagcgggagccggacgacgggggcgggcagacgtacgtcgtccgcagcgacgacggccagaccgcgtaccgcttcaccgccaccgtccgcgccctggaccactggcaggtggacgccgcctccgtcacccgccaccgcgacggcgcggaactcccgctcgccgcgctggacttcttcatcgagctgaagcagaccctgggcctgagcgacgagatcctcccggtctacctggaggagatctcctccaccctctccggcacctgctacaaactgaccaagccgcagctcagctccgccgagctggcccggagcggagacttccaggccgtcgagacgggcatgaccgagggccacccgtgcttcgtcgccaacaacgggcgcctcggcttcggcatccacgagtacctgtcgtacgcccccgagaccgcgagcccggtccggctggtgtggctggccgcgcaccgctcgcgggcggcgttcacggcgggcgtgggcatcgagtacgagtccttcgtccgggacgagctgggcgcggccaccgtcgaccgcttccacggcgtgctgcgcgggcgcggcctggacccggccgactacctgctcatcccggtccacccctggcagtggtggaacaagctcaccgtcaccttcgccgccgaggtcgcccgcgggcacctggtgtgcctgggcgagggcgacgacgagtacctggcccagcagtccatccgcaccttcttcaacgcctcgcaccccgggaagcactacgtgaagactgccctgtccgtcctcaacatgggcttcatgcgcggtctgtcggcggcgtacatggaggccactccggccatcaacgactggctcgcccggctgatcgagggcgacccggtgctgaaggagacggggctgagcatcatccgggagcgggcggccgtcggctaccggcacctggagtacgagcaggccaccgaccgctactcgccctaccgcaagatgctggcggcgctgtggcgggagagcccggtgccgtccatccgggagggcgagacgctcgccaccatggcctccctggtccaccaggaccacgagggcgcctccttcgcgggcgcgctgatcgagcggtccggactcacgcccaccgagtggctgcggcactacctgcgggcctactacgtcccgctgctgcacagcttctacgcctacgacctggtgtacatgccgcacggcgagaacgtgatcctggtgctggcggacggggtggtgcggcgggcggtctacaaggacatcgccgaggagatcgcggtgatggacccggacgcggtgctgccgccggaggtctcccgcatcgcggtggacgtgccggacgacaagaagctcctgtcgatcttcacggacgtcttcgactgcttcttccgcttcctggccgcgaacctggcggaggaggggatcgtcacggaggacgccttctggcggacggtcgcggaggtcacccgggagtaccaggagtcggtgccggagctggccgacaagttcgagcggtacgacatgttcgcgcccgagttcgccctgtcctgcctcaaccggctccagctgcgcgacaaccggcagatggtggacctggccgacccgtccggcgcgctccagctcgtcggcaccctgaagaaccccctggcaggccggtag

SEQ ID NO: 15 - protein, 480 aa, Streptomyces violaceoruber
MRSHLLNDTTAEQYRRSVTEGVERVAAKLATTDRPFTGVTVDALSPRIDAIDLDEPLHDTAAVLDELEDVYLRDAVYFHHPRYLAHLNCPVVIPALLGEAVLSAVNSSLDTWDQSAGGTLIERKLIDWTCARIGLGPAADGVFTSGGTQSNLQALLLAREEAKAEDFADLRIFASEASHFSVRKSAKLLGLGPDAVVSIPVDRDKRMQTVALARELERCARDGLVPMAVVATGGTTDFGSIDPLPEIAGLCEQYGVWMHVDAAYGCGLLASLKYRDRITGIERADSVTVDYHKSFFQPVSSSAVLVRDAATLRHATYHAEYLNPRRMVQERIPNQVDKSLQTTRRFDALKLWMTLRVMGADGIGVLFDEVCDLAAEGWKLLAADPRFDVVVQPSLSTLVFRHIPADVTDPAEIDRANLYARKALFASGDAVVAGTKVAGRHYLKFTLLNPETTPADIAAVLDLIAGHAEQYLGDSLDRAS

SEQ ID NO: 16 - protein, 420 aa, Streptomyces violaceoruber
LTALPEASAPYDFVGIGLGPFNLGLACLTEPVAELNGVFLESKPDFEWHAGMFLDGAHLQTPFMSDLVTLADPTSPYSFLNYLKEQGRLYSFYIRENFYPLRVEYDDYCRWAARKLSSVRFSTTVTEVTYDEREELYAVATTSGDTYRARRLVLGTGTPPHIPDACRGLAGDFLHNSRYVRHRAELVKKKSITLVGSGQSAAEIYQDLLSEIDVHGYGLNWVTRSPRFFPLEYTKLTLEMTSPEYVDYYHALPEDTRYRLTAEQKGLFKGIDGDLINEIFDLLYQKRLGGPVPTRLLTNSALTSARYADGTYTLGFRQEEQGTDFEIETEGLVLATGYRYTEPEFLKPVRDRLRYDSRGNFDIGRNYAVDVTGGGVFLQNAGVHAHSVTSPDLGMGAYRNSCIVRELLGREYYPVEQSIA

SEQ ID NO: 17 - protein, 184 aa, Streptomyces violaceoruber
MSRLSTTTPVGALTLRPVDPLTDAVLLHGWLTHPKSAFWMMQDARLVDVERAYMELAADEHQQAHLGLHDGVPAFLTERYDPAHRELVGLYEPEPGDVGMHFLVAPTDRPVHGFTRAVITTVMTELFADPATRRVVVEPDVTNTAVHALNAAVGFVPEREIQKPEKKALLSFCTREQFAKAVSA

SEQ ID NO: 18 - protein, 595 aa, Streptomyces violaceoruber
MSLADAVAHLTPERWEEANRLLVRKALAEFTHERLLTPEREPDDGGGQTYVVRSDDGQTAYRFTATVRALDHWQVDAASVTRHRDGAELPLAALDFFIELKQTLGLSDEILPVYLEEISSTLSGTCYKLTKPQLSSAELARSGDFQAVETGMTEGHPCFVANNGRLGFGIHEYLSYAPETASPVRLVWLAAHRSRAAFTAGVGIEYESFVRDELGAATVDRFHGVLRGRGLDPADYLLIPVHPWQWWNKLTVTFAAEVARGHLVCLGEGDDEYLAQQSIRTFFNASHPGKHYVKTALSVLNMGFMRGLSAAYMEATPAINDWLARLIEGDPVLKETGLSIIRERAAVGYRHLEYEQATDRYSPYRKMLAALWRESPVPSIREGETLATMASLVHQDHEGASFAGALIERSGLTPTEWLRHYLRAYYVPLLHSFYAYDLVYMPHGENVILVLADGVVRRAVYKDIAEEIAVMDPDAVLPPEVSRIAVDVPDDKKLLSIFTDVFDCFFRFLAANLAEEGIVTEDAFWRTVAEVTREYQESVPELADKFERYDMFAPEFALSCLNRLQLRDNRQMVDLADPSGALQLVGTLKNPLAGR

SEQ ID NO: 19 - DNA, 5120 bp, synthetic construct
  misc_feature 1..5120: note = Vector Construct
  source 1..5120: mol_type = other DNA
acaggaaacagaattaattaagcttccccaccggaggacccccccatgcgctcgcacctgctcaacgacaccaccgcggagcagtaccgccgttccgtgaccgaaggagtcgagcgggtggccgccaaactcgccaccaccgaccgcccgttcaccggcgtcacggtcgacgccctctccccccgcatcgacgcgatcgacctcgacgagccgctgcacgacaccgccgcggtcctcgacgagctggaggacgtctacctccgcgacgccgtctacttccaccacccccgctacctcgcccacctcaactgcccggtcgtcataccggcgctgctcggtgaagcggtcctgtccgccgtcaactcctccctggacacctgggaccagtcggccggcggcacgctcatcgagcggaagctgatcgactggacctgcgcccgcatcggcctcggcccggcggccgacggcgtgttcacctccggcggcacccagtccaacctccaggcgctgctcctcgcccgcgaggaggcgaaggccgaggacttcgccgacctgcggatcttcgcctccgaggccagccacttcagcgtcaggaagtccgcgaaactgctcggcctcggccccgacgccgtcgtgtcgatcccggtcgaccgcgacaagcggatgcagaccgtcgccctcgcccgcgagctggagcgctgcgcgcgggacggcctggtccccatggccgtcgtcgccaccggcggcaccaccgacttcggctcgatcgacccgctgccggagatcgccgggctgtgcgagcagtacggcgtgtggatgcacgtcgacgcggcctacggctgcgggctgctcgcctccctgaagtaccgggaccgcatcaccggcatcgagcgggccgactcggtcaccgtggactaccacaagtccttcttccagccggtgagttcgtcggccgtgctggtccgggacgcggccaccctgcgccacgccacctaccacgcggagtacctcaacccgcgccgcatggtgcaggaacgtatccccaaccaggtggacaagtccctccagaccacccgccgcttcgacgcgctcaagctgtggatgacgctgcgcgtgatgggcgccgacggcatcggcgtcctcttcgacgaggtgtgcgacctggccgccgagggctggaaactgctcgccgccgacccgcgcttcgacgtcgtggtccagccgtcgctgtccacgctggtcttccgccacatcccggcggacgtcaccgatcccgccgagatcgaccgcgccaacctgtacgcccgcaaggccctgttcgcgtccggcgacgccgtggtcgcgggcaccaaggtcgccggtcgccactacctgaagttcaccctgctcaaccccgagacgaccccggccgacatcgccgccgtcctcgacctgatcgccggccacgccgagcagtacctgggagactcccttgaccgcgcttcctgaagccagcgccccgtacgacttcgtgggcatcggcctcggccccttcaacctcggcctcgcctgcctcaccgagcccgtcgccgagctgaacggcgtcttcctggagtccaagccggacttcgagtggcacgccgggatgttcctggacggcgcccacctccagaccccgttcatgtcggacctggtgacgctcgccgacccgacctcgccgtactccttcctcaactacctgaaggagcagggccggctgtactccttctacatccgggagaacttctacccgctgcgggtcgagtacgacgactactgccgctgggccgcgcgcaagctgagcagcgtccgcttctccaccacggtcaccgaggtcacgtacgacgagcgcgaggagctgtacgccgtcgccaccacgtccggcgacacctaccgcgcccgccgcctcgtcctcggcaccggcaccccgccgcacatcccggacgcctgccggggcctggccggcgacttcctccacaactcccggtacgtgcggcaccgggcggagctggtgaagaagaagtcgatcacgctggtcggcagcggccagtccgccgccgagatctaccaggacctgctgagcgagatcgacgtccacggctacgggctgaactgggtgacccgctccccgcggttcttcccgctcgaatacaccaagctcaccctggagatgacgtccccggagtacgtggactactaccacgcgctgcccgaggacacccgctaccgcctcacggccgagcagaagggcctgttcaagggcatcgacggcgacctgatcaacgagatcttcgacctgctctaccagaaacggctcggcggccccgtccccacccgcctgctcaccaactcggcgctgaccagcgcccggtatgcggacggcacctacacgctcgggttccgccaggaggagcagggcacggacttcgagatcgagaccgagggcctggtcctcgccaccggctaccggtacaccgagccggagttcctcaagcccgtccgggaccggctgcgctacgactcccgcggcaacttcgacatcggccgcaactacgccgtcgacgtcacgggaggcggcgtgttcctccagaacgcgggggtccacgcgcacagcgtcaccagccccgacctgggcatgggcgcctaccgcaacagctgcatcgtccgggagctgctcggacgcgagtactacccggtcgagcagtcgatcgcgttccaggagttcgccgtatgagccgcttgagcaccaccacccccgtcggggcactgaccctgcgccccgtcgacccgctgacggacgccgtactgctgcacggctggctcacccaccccaagtccgcgttctggatgatgcaggacgcccggctggtggacgtcgagcgggcctacatggagctggccgccgacgagcaccagcaggcccacctcggcctgcacgacggggtcccggccttcctgacggagcgctacgaccccgcccaccgcgaactggtcgggctgtacgagcccgagccgggcgacgtcggcatgcacttcctggtcgcgcccaccgaccggcccgtgcacggcttcacccgcgccgtgatcaccaccgtgatgacggagctgttcgccgacccggcgacccggcgggtcgtcgtcgaaccggacgtcaccaacaccgccgtgcacgccctgaacgcagccgtcggattcgtgcccgagcgcgagatccagaagccggagaagaaggccttgctgagcttctgcacccgcgagcagttcgcgaaggcggtgtccgcatgagcctcgccgacgccgtcgcccacctgacccccgaacgctgggaggaggccaaccgcctcctggtccgcaaggcgctggccgagttcacccacgagcggctgctgacccccgagcgggagccggacgacgggggcgggcagacgtacgtcgtccgcagcgacgacggccagaccgcgtaccgcttcaccgccaccgtccgcgccctggaccactggcaggtggacgccgcctccgtcacccgccaccgcgacggcgcggaactcccgctcgccgcgctggacttcttcatcgagctgaagcagaccctgggcctgagcgacgagatcctcccggtctacctggaggagatctcctccaccctctccggcacctgctacaaactgaccaagccgcagctcagctccgccgagctggcccggagcggagacttccaggccgtcgagacgggcatgaccgagggccacccgtgcttcgtcgccaacaacgggcgcctcggcttcggcatccacgagtacctgtcgtacgcccccgagaccgcgagcccggtccggctggtgtggctggccgcgcaccgctcgcgggcggcgttcacggcgggcgtgggcatcgagtacgagtccttcgtccgggacgagctgggcgcggccaccgtcgaccgcttccacggcgtgctgcgcgggcgcggcctggacccggccgactacctgctcatcccggtccacccctggcagtggtggaacaagctcaccgtcaccttcgccgccgaggtcgcccgcgggcacctggtgtgcctgggcgagggcgacgacgagtacctggcccagcagtccatccgcaccttcttcaacgcctcgcaccccgggaagcactacgtgaagactgccctgtccgtcctcaacatgggcttcatgcgcggtctgtcggcggcgtacatggaggccactccggccatcaacgactggctcgcccggctgatcgagggcgacccggtgctgaaggagacggggctgagcatcatccgggagcgggcggccgtcggctaccggcacctggagtacgagcaggccaccgaccgctactcgccctaccgcaagatgctggcggcgctgtggcgggagagcccggtgccgtccatccgggagggcgagacgctcgccaccatggcctccctggtccaccaggaccacgagggcgcctccttcgcgggcgcgctgatcgagcggtccggactcacgcccaccgagtggctgcggcactacctgcgggcctactacgtcccgctgctgcacagcttctacgcctacgacctggtgtacatgccgcacggcgagaacgtgatcctggtgctggcggacggggtggtgcggcgggcggtctacaaggacatcgccgaggagatcgcggtgatggacccggacgcggtgctgccgccggaggtctcccgcatcgcggtggacgtgccggacgacaagaagctcctgtcgatcttcacggacgtcttcgactgcttcttccgcttcctggccgcgaacctggcggaggaggggatcgtcacggaggacgccttctggcggacggtcgcggaggtcacccgggagtaccaggagtcggtgccggagctggccgacaagttcgagcggtacgacatgttcgcgcccgagttcgccctgtcctgcctcaaccggctccagctgcgcgacaaccggcagatggtggacctggccgacccgtccggcgcgctccagctcgtcggcaccctgaagaaccccctggcaggccggtagccaaccggaagcttgcatgcctgcaggtcgact

SEQ ID NO: 20 - DNA, 11677 bp, synthetic construct
  misc_feature 1..11677: note = Vector Construct
  source 1..11677: mol_type = other DNA
aattaagcttccccaccggaggacccccccatgcgctcgcacctgctcaacgacaccaccgcggagcagtaccgccgttccgtgaccgaaggagtcgagcgggtggccgccaaactcgccaccaccgaccgcccgttcaccggcgtcacggtcgacgccctctccccccgcatcgacgcgatcgacctcgacgagccgctgcacgacaccgccgcggtcctcgacgagctggaggacgtctacctccgcgacgccgtctacttccaccacccccgctacctcgcccacctcaactgcccggtcgtcataccggcgctgctcggtgaagcggtcctgtccgccgtcaactcctccctggacacctgggaccagtcggccggcggcacgctcatcgagcggaagctgatcgactggacctgcgcccgcatcggcctcggcccggcggccgacggcgtgttcacctccggcggcacccagtccaacctccaggcgctgctcctcgcccgcgaggaggcgaaggccgaggacttcgccgacctgcggatcttcgcctccgaggccagccacttcagcgtcaggaagtccgcgaaactgctcggcctcggccccgacgccgtcgtgtcgatcccggtcgaccgcgacaagcggatgcagaccgtcgccctcgcccgcgagctggagcgctgcgcgcgggacggcctggtccccatggccgtcgtcgccaccggcggcaccaccgacttcggctcgatcgacccgctgccggagatcgccgggctgtgcgagcagtacggcgtgtggatgcacgtcgacgcggcctacggctgcgggctgctcgcctccctgaagtaccgggaccgcatcaccggcatcgagcgggccgactcggtcaccgtggactaccacaagtccttcttccagccggtgagttcgtcggccgtgctggtccgggacgcggccaccctgcgccacgccacctaccacgcggagtacctcaacccgcgccgcatggtgcaggaacgtatccccaaccaggtggacaagtccctccagaccacccgccgcttcgacgcgctcaagctgtggatgacgctgcgcgtgatgggcgccgacggcatcggcgtcctcttcgacgaggtgtgcgacctggccgccgagggctggaaactgctcgccgccgacccgcgcttcgacgtcgtggtccagccgtcgctgtccacgctggtcttccgccacatcccggcggacgtcaccgatcccgccgagatcgaccgcgccaacctgtacgcccgcaaggccctgttcgcgtccggcgacgccgtggtcgcgggcaccaaggtcgccggtcgccactacctgaagttcaccctgctcaaccccgagacgaccccggccgacatcgccgccgtcctcgacctgatcgccggccacgccgagcagtacctgggagactcccttgaccgcgcttcctgaagccagcgccccgtacgacttcgtgggcatcggcctcggccccttcaacctcggcctcgcctgcctcaccgagcccgtcgccgagctgaacggcgtcttcctggagtccaagccggacttcgagtggcacgccgggatgttcctggacggcgcccacctccagaccccgttcatgtcggacctggtgacgctcgccgacccgacctcgccgtactccttcctcaactacctgaaggagcagggccggctgtactccttctacatccgggagaacttctacccgctgcgggtcgagtacgacgactactgccgctgggccgcgcgcaagctgagcagcgtccgcttctccaccacggtcaccgaggtcacgtacgacgagcgcgaggagctgtacgccgtcgccaccacgtccggcgacacctaccgcgcccgccgcctcgtcctcggcaccggcaccccgccgcacatcccggacgcctgccggggcctggccggcgacttcctccacaactcccggtacgtgcggcaccgggcggagctggtgaagaagaagtcgatcacgctggtcggcagcggccagtccgccgccgagatctaccaggacctgctgagcgagatcgacgtccacggctacgggctgaactgggtgacccgctccccgcggttcttcccgctcgaatacaccaagctcaccctggagatgacgtccccggagtacgtggactactaccacgcgctgcccgaggacacccgctaccgcctcacggccgagcagaagggcctgttcaagggcatcgacggcgacctgatcaacgagatcttcgacctgctctaccagaaacggctcggcggccccgtccccacccgcctgctcaccaactcggcgctgaccagcgcccggtatgcggacggcacctacacgctcgggttccgccaggaggagcagggcacggacttcgagatcgagaccgagggcctggtcctcgccaccggctaccggtacaccgagccggagttcctcaagcccgtccgggaccggctgcgctacgactcccgcggcaacttcgacatcggccgcaactacgccgtcgacgtcacgggaggcggcgtgttcctccagaacgcgggggtccacgcgcacagcgtcaccagccccgacctgggcatgggcgcctaccgcaacagctgcatcgtccgggagctgctcggacgcgagtactacccggtcgagcagtcgatcgcgttccaggagttcgccgtatgagccgcttgagcaccaccacccccgtcggggcactgaccctgcgccccgtcgacccgctgacggacgccgtactgctgcacggctggctcacccaccccaagtccgcgttctggatgatgcaggacgcccggctggtggacgtcgagcgggcctacatggagctggccgccgacgagcaccagcaggcccacctcggcctgcacgacggggtcccggccttcctgacggagcgctacgaccccgcccaccgcgaactggtcgggctgtacgagcccgagccgggcgacgtcggcatgcacttcctggtcgcgcccaccgaccggcccgtgcacggcttcacccgcgccgtgatcaccaccgtgatgacggagctgttcgccgacccggcgacccggcgggtcgtcgtcgaaccggacgtcaccaacaccgccgtgcacgccctgaacgcagccgtcggattcgtgcccgagcgcgagatccagaagccggagaagaaggccttgctgagcttctgcacccgcgagcagttcgcgaaggcggtgtccgcatgagcctcgccgacgccgtcgcccacctgacccccgaacgctgggaggaggccaaccgcctcctggtccgcaaggcgctggccgagttcacccacgagcggctgctgacccccgagcgggagccggacgacgggggcgggcagacgtacgtcgtccgcagcgacgacggccagaccgcgtaccgcttcaccgccaccgtccgcgccctggaccactggcaggtggacgccgcctccgtcacccgccaccgcgacggcgcggaactcccgctcgccgcgctggacttcttcatcgagctgaagcagaccctgggcctgagcgacgagatcctcccggtctacctggaggagatctcctccaccctctccggcacctgctacaaactgaccaagccgcagctcagctccgccgagctggcccggagcggagacttccaggccgtcgagacgggcatgaccgagggccacccgtgcttcgtcgccaacaacgggcgcctcggcttcggcatccacgagtacctgtcgtacgcccccgagaccgcgagcccggtccggctggtgtggctggccgcgcaccgctcgcgggcggcgttcacggcgggcgtgggcatcgagtacgagtccttcgtccgggacgagctgggcgcggccaccgtcgaccgcttccacggcgtgctgcgcgggcgcggcctggacccggccgactacctgctcatcccggtccacccctggcagtggtggaacaagctcaccgtcaccttcgccgccgaggtcgcccgcgggcacctggtgtgcctgggcgagggcgacgacgagtacctggcccagcagtccatccgcaccttcttcaacgcctcgcaccccgggaagcactacgtgaagactgccctgtccgtcctcaacatgggcttcatgcgcggtctgtcggcggcgtacatggaggccactccggccatcaacgactggctcgcccggctgatcgagggcgacccggtgctgaaggagacggggctgagcatcatccgggagcgggcggccgtcggctaccggcacctggagtacgagcaggccaccgaccgctactcgccctaccgcaagatgctggcggcgctgtggcgggagagcccggtgccgtccatccgggagggcgagacgctcgccaccatggcctccctggtccaccaggaccacgagggcgcctccttcgcgggcgcgctgatcgagcggtccggactcacgcccaccgagtggctgcggcactacctgcgggcctactacgtcccgctgctgcacagcttctacgcctacgacctggtgtacatgccgcacggcgagaacgtgatcctggtgctggcggacggggtggtgcggcgggcggtctacaaggacatcgccgaggagatcgcggtgatggacccggacgcggtgctgccgccggaggtctcccgcatcgcggtggacgtgccggacgacaagaagctcctgtcgatcttcacggacgtcttcgactgcttcttccgcttcctggccgcgaacctggcggaggaggggatcgtcacggaggacgccttctggcggacggtcgcggaggtcacccgggagtaccaggagtcggtgccggagctggccgacaagttcgagcggtacgacatgttcgcgcccgagttcgccctgtcctgcctcaaccggctccagctgcgcgacaaccggcagatggtggacctggccgacccgtccggcgcgctccagctcgtcggcaccctgaagaaccccctggcaggccggtagccaaccggaagcttgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 21 - DNA, 1443 bp, Streptomyces pilosus
  source 1..1443: mol_type = genomic DNA
atgcgctcgcacctgctcaacgacaccaccgcggagcactaccgccgctccgtgaccgagggagtcgagcgggtggcggccaaactcgccaccacagcccggccgttcaccggcgtcacggtcgacgccctcgcgccccgcatcgacgcgatcgacctcgaccggcccctgcacgacaccaccgcggtactggacgaactggaggacgtctacctccgggacgcgatctacttccaccacccgcgctacctcgcccacctcaactgcccggtcgtcatcccggccctgctcggcgaggcggtgctctccgccgtcaactcctccctggacacctgggaccagtcggcgggcggcaccctcatcgagcgcaagctcatcgactggacgaccgcccgcatcggcctcggccccgccgccgacggcgtgttcacctccggcggcacccagtccaacctccaggcgctgctgctcgcccgcgaggaggccaagaccggtgacctggcgaaactgcgcgtcttcgcctccgaggtcagccacttcagcgtgaagaagtccgcgaaactgctcggcctcgggcccgacgccgtgatctcgatcccagtcgaccacgacaagcggctgcagaccgtcgccctcgcccgcgagctggagcggtgcgtcgaggcgggcctcgtccccatggccgtcgtcgccaccgccggcaccaccgacttcggctccatcgacccgctgcccgggatcgccgaactgtgcgggcggtacggcgcctggatgcacgtcgacgccgcctacggctgcggtctgctcgcctcactgaagtaccgcgaccgcatcgccggcatcgagcacgccgactccgtcaccgtcgactaccacaagtccttcttccagcccgtcagttcgtcggccgtactggtccgcgacaccgccacgctgcggcacgccacctaccacgccgagtacctcaacccgcgccgcatggtgatcgagcgcatccccaaccaggtcgacaagtccctgcagaccacccgccgcttcgacgccctgaaactgtggatgacgctgcgcgtgatgggtgccgacggcatcggcggcctcttcgacgaggtgtgcgacctggccgcggagggctggaagctgctcgccgccgacccgcgcttcgacgtcgtggtcgcgcccagcctctccaccctcgtcttccgctacatcccggcggccgtcaccgaccccgccgagatcgaccgcgccaacctctacgcccgcaaggccctcttcgcctccggcgacgcggtcgtcgccggcaccaaggtggccgggcgccactacctgaagttcaccctgctgaaccccgagaccacgacggacgacatctccgccgtcctcgatctgatcgccggccacgccgagcagtacctgggagagtcccttgaccgcgcttgctga

SEQ ID NO: 22 - DNA, 1293 bp, Streptomyces pilosus
  source 1..1293: mol_type = genomic DNA
ttgaccgcgcttgctgaagccccggaaaccacctacgacttcgtgggcgtcgggctgggccccttcaacctcggcctcgcctgcctcaccgagccgatatccgaactgaacggcgtcttcctggagtccaagccggacttcgcctggcacgccgggatgttcctcgacggcgcccacctccagacccccttcatgtcggacctggtcaccctcgccgaccccacctccccctactccttcctcaactacctgaaggagaagggccggctgtactcgttctacatacgcgagaacttctacccgctgcgggtcgagtacgacgactactgccgctgggccgccgccaagctgagcagcatccgcttcggcacgacggtcaccgaggtgacctacgacgaggccgccgagctgtacgcggtcgcgacggcggccggtgagacgtaccgcgcccggcacctggtgctcggcaccggcaccgtgccgttcctcccggagccgtgccgcgggctggacggcggcctcttccacaacgcgcagtacatgcagcgcaaggcggaactgctggagaagaagtcggtcacggtcgtcggcagcggtcagagcgccgccgagatctactacgagctgctctccgagatcgacgtccacggctaccagctcaactgggtcacgcgttcgccgcggttcttcccgctggagtacaccaagctgaccctggagatgacctccccggactacatcgactacttccgcgccctgcccgaggagacccgctaccggctggagaagcagcagaagggcctgttcaagggcatcaactcggagctgatcgacgccatcttcgacctgctctaccagaagaacgtcgagagcggggaccgtccggtacccacccggctgctcaccaactccagcctcaccggcgcgcggtaccaggacggcgcgtacacgctcggcttccaccaggacgagcagggcaaggacttcgagatccgcaccgagggcctggtgctggccaccggctaccactacgagccgccggcgttcctcgacccggtgcgcgagcggatccgcttcgacggccacggccgtttcgacgtcgcccgcaactacgccatcgacgtcaccggccgggggatcttcctgcagaacgcgggcgtccacacgcacagcatcaccagccccgacctgggcatgggcccgtaccgcaacgcgtccatcatccgtgagctgctgggcaccgagtactacccggtcgagaagagcatcgcgttccaggagttcgccgtatga

SEQ ID NO: 23 - DNA, 540 bp, Streptomyces pilosus
  source 1..540: mol_type = genomic DNA
atgagcatcggtacgttcaccgtccgccccctggacccgctcgaggacgccgagctgctgcaccgctgggtcaccgaccccaaggcggcgttctggatgatgcaggacgccgcgctccaggacgtcgagcgcgagtacctgcgcatcgccgcccacgagcaccaccacgcctacctgggcctgcacgacggcgaacccgccttcctgatggagaagtacgacccccggtacgtcgaactcgtcggcctgtacgaccccgagcccggcgacgtcggcatgcacttcctggtcgcccccaccgaccgccccgtccacggcttcacccgggcggtcatcaccgccgtgatggaggagctgttcgccgaccccgccacccggcgggtggtggtcgaaccggacgtcggcaacaaggccgtgcacgccctcaacgaggccgtgggcttcgtgcccgagcgggagatcgacaagccggagaagcgcgcgctgctgagcttctgcacgcgtgaacagttcgaggccgcccgaggagcatccgcatga

SEQ ID NO: 24 - DNA, 1773 bp, Streptomyces pilosus
  source 1..1773: mol_type = genomic DNA
atgaccctgtccgacgccgtggcgcacctgtccccccaccgctgggcgcgggccaaccgcctgctgatccgcaaggcgctcgccgagttcgcccacgagcggctcatcacgccgcaggactccggcgacggccggtacgccgtccacagcgacgacggccggacccggtacacgttcaccgctgcgcggcgcgccctggaccactggcacatcgaccccgagtcgatcacccgccaccgcgacgacgccgaacgcgagctcgcggccctggacttcttcatcgagctgcagaagtccctgggcctgagcgaggagattctgccggtgtacctggaggagatctcctccaccctctccggcacctgctacaagctcaccaagccgcaggtgcgggccggagagctgatcgacgccggcttccaggccgtcgagaccggcatgaccgaggggcacccctgcttcgtcgccaacaacgggcggctcggcttcggcgtcgacgagtaccgcgcgtacgcgccggaggcggcggccccggtcaagctggtgtggctggccgcccaccgctcgcgggccgcgttcacggcgggcgtgggcatcgagtacgagtcgttcgtacggcaggaactgggcgaggagaccgtcgagggcttccacgacgtcctgcgcgcgcaggacctcgaccccggcgactacctcctgatacccgtccacccctggcagtggtggaacaagctgtccgtcaccttcgccgccgagatcgcccgccggcacctggtgtgcctgggcgagggcgacgacgagtacctggcccagcagtccatccgcaccttcttcaaccggtcgcaccccgggaagcactacgtgaagacggccctgtccgtcctcaacatgggcttcatgcgcgggctctccgccgcctacatggaggcgaccccggcgatcaacgactggctggcgcagctcgtcgacagcgacccggtgctgcgcgcgaccgggctgtcgatcatccgggagcgggccgccgtgggctaccggcacctggagtacgagcaggccacggaccgctactcgccgtaccgcaagatgctggccgcgctgtggcgggagagcccggtgccgtccctggaggacggcgagtccctcgccaccatggcctcgctggtccatgtcgaccacgagggcgcctccgtggcggccgcgctgatcgcacgctccgggctggcccccaccgagtggctgcgccactacctgcgcgcctactacgtgccgctgctgcacagcttctacgcctacgacctggcctacatgccgcacggcgagaacgtgatcctcgtgctgcgggacggcgtggtgcggcgcgcgatctacaaggacatcgccgaggagatcgtggtgatggacgccgacgcggtgctgccgccggaggtggcccgcatccgggcggaggtgccggacgacacgaagctgctgtcgatcttcacggacgtcttcgactgcttcttccgcttcctcgccgcgaacctggccgaggagggcgtcctggacgaggacggcttctggcgcacggtcgcggaggtcacccgcgagtaccaggcgtcggtgccggaactcgccgacaagttcgcccggtacgacatgttcgccccggagttcgccctgtcctgcctgaaccgcctgcagttgcgcaacaaccggcagatggtcgacctgtcggacccctccggcgcgctccagctggtcggcaccctgaagaaccccctcgcggagttctga

SEQ ID NO: 25 - protein, 480 aa, Streptomyces pilosus
MRSHLLNDTTAEHYRRSVTEGVERVAAKLATTARPFTGVTVDALAPRIDAIDLDRPLHDTTAVLDELEDVYLRDAIYFHHPRYLAHLNCPVVIPALLGEAVLSAVNSSLDTWDQSAGGTLIERKLIDWTTARIGLGPAADGVFTSGGTQSNLQALLLAREEAKTGDLAKLRVFASEVSHFSVKKSAKLLGLGPDAVISIPVDHDKRLQTVALARELERCVEAGLVPMAVVATAGTTDFGSIDPLPGIAELCGRYGAWMHVDAAYGCGLLASLKYRDRIAGIEHADSVTVDYHKSFFQPVSSSAVLVRDTATLRHATYHAEYLNPRRMVIERIPNQVDKSLQTTRRFDALKLWMTLRVMGADGIGGLFDEVCDLAAEGWKLLAADPRFDVVVAPSLSTLVFRYIPAAVTDPAEIDRANLYARKALFASGDAVVAGTKVAGRHYLKFTLLNPETTTDDISAVLDLIAGHAEQYLGESLDRAC

SEQ ID NO: 26 - protein, 430 aa, Streptomyces pilosus
LTALAEAPETTYDFVGVGLGPFNLGLACLTEPISELNGVFLESKPDFAWHAGMFLDGAHLQTPFMSDLVTLADPTSPYSFLNYLKEKGRLYSFYIRENFYPLRVEYDDYCRWAAAKLSSIRFGTTVTEVTYDEAAELYAVATAAGETYRARHLVLGTGTVPFLPEPCRGLDGGLFHNAQYMQRKAELLEKKSVTVVGSGQSAAEIYYELLSEIDVHGYQLNWVTRSPRFFPLEYTKLTLEMTSPDYIDYFRALPEETRYRLEKQQKGLFKGINSELIDAIFDLLYQKNVESGDRPVPTRLLTNSSLTGARYQDGAYTLGFHQDEQGKDFEIRTEGLVLATGYHYEPPAFLDPVRERIRFDGHGRFDVARNYAIDVTGRGIFLQNAGVHTHSITSPDLGMGPYRNASIIRELLGTEYYPVEKSIAFQEFAV

SEQ ID NO: 27 - protein, 179 aa, Streptomyces pilosus
MSIGTFTVRPLDPLEDAELLHRWVTDPKAAFWMMQDAALQDVEREYLRIAAHEHHHAYLGLHDGEPAFLMEKYDPRYVELVGLYDPEPGDVGMHFLVAPTDRPVHGFTRAVITAVMEELFADPATRRVVVEPDVGNKAVHALNEAVGFVPEREIDKPEKRALLSFCTREQFEAARGASA

SEQ ID NO: 28 - protein, 590 aa, Streptomyces pilosus
MTLSDAVAHLSPHRWARANRLLIRKALAEFAHERLITPQDSGDGRYAVHSDDGRTRYTFTAARRALDHWHIDPESITRHRDDAERELAALDFFIELQKSLGLSEEILPVYLEEISSTLSGTCYKLTKPQVRAGELIDAGFQAVETGMTEGHPCFVANNGRLGFGVDEYRAYAPEAAAPVKLVWLAAHRSRAAFTAGVGIEYESFVRQELGEETVEGFHDVLRAQDLDPGDYLLIPVHPWQWWNKLSVTFAAEIARRHLVCLGEGDDEYLAQQSIRTFFNRSHPGKHYVKTALSVLNMGFMRGLSAAYMEATPAINDWLAQLVDSDPVLRATGLSIIRERAAVGYRHLEYEQATDRYSPYRKMLAALWRESPVPSLEDGESLATMASLVHVDHEGASVAAALIARSGLAPTEWLRHYLRAYYVPLLHSFYAYDLAYMPHGENVILVLRDGVVRRAIYKDIAEEIVVMDADAVLPPEVARIRAEVPDDTKLLSIFTDVFDCFFRFLAANLAEEGVLDEDGFWRTVAEVTREYQASVPELADKFARYDMFAPEFALSCLNRLQLRNNRQMVDLSDPSGALQLVGTLKNPLAEF

SEQ ID NO: 29 - DNA, 5102 bp, synthetic construct
  misc_feature 1..5102: note = Vector Construct
  source 1..5102: mol_type = other DNA
acaggaaacagaattaattaagctttgcaggaaggagatatagatatgcgctcgcacctgctcaacgacaccaccgcggagcactaccgccgctccgtgaccgagggagtcgagcgggtggcggccaaactcgccaccacagcccggccgttcaccggcgtcacggtcgacgccctcgcgccccgcatcgacgcgatcgacctcgaccggcccctgcacgacaccaccgcggtactggacgaactggaggacgtctacctccgggacgcgatctacttccaccacccgcgctacctcgcccacctcaactgcccggtcgtcatcccggccctgctcggcgaggcggtgctctccgccgtcaactcctccctggacacctgggaccagtcggcgggcggcaccctcatcgagcgcaagctcatcgactggacgaccgcccgcatcggcctcggccccgccgccgacggcgtgttcacctccggcggcacccagtccaacctccaggcgctgctgctcgcccgcgaggaggccaagaccggtgacctggcgaaactgcgcgtcttcgcctccgaggtcagccacttcagcgtgaagaagtccgcgaaactgctcggcctcgggcccgacgccgtgatctcgatcccagtcgaccacgacaagcggctgcagaccgtcgccctcgcccgcgagctggagcggtgcgtcgaggcgggcctcgtccccatggccgtcgtcgccaccgccggcaccaccgacttcggctccatcgacccgctgcccgggatcgccgaactgtgcgggcggtacggcgcctggatgcacgtcgacgccgcctacggctgcggtctgctcgcctcactgaagtaccgcgaccgcatcgccggcatcgagcacgccgactccgtcaccgtcgactaccacaagtccttcttccagcccgtcagttcgtcggccgtactggtccgcgacaccgccacgctgcggcacgccacctaccacgccgagtacctcaacccgcgccgcatggtgatcgagcgcatccccaaccaggtcgacaagtccctgcagaccacccgccgcttcgacgccctgaaactgtggatgacgctgcgcgtgatgggtgccgacggcatcggcggcctcttcgacgaggtgtgcgacctggccgcggagggctggaagctgctcgccgccgacccgcgcttcgacgtcgtggtcgcgcccagcctctccaccctcgtcttccgctacatcccggcggccgtcaccgaccccgccgagatcgaccgcgccaacctctacgcccgcaaggccctcttcgcctccggcgacgcggtcgtcgccggcaccaaggtggccgggcgccactacctgaagttcaccctgctgaaccccgagaccacgacggacgacatctccgccgtcctcgatctgatcgccggccacgccgagcagtacctgggagagtcccttgaccgcgcttgctgaagccccggaaaccacctacgacttcgtgggcgtcgggctgggccccttcaacctcggcctcgcctgcctcaccgagccgatatccgaactgaacggcgtcttcctggagtccaagccggacttcgcctggcacgccgggatgttcctcgacggcgcccacctccagacccccttcatgtcggacctggtcaccctcgccgaccccacctccccctactccttcctcaactacctgaaggagaagggccggctgtactcgttctacatacgcgagaacttctacccgctgcgggtcgagtacgacgactactgccgctgggccgccgccaagctgagcagcatccgcttcggcacgacggtcaccgaggtgacctacgacgaggccgccgagctgtacgcggtcgcgacggcggccggtgagacgtaccgcgcccggcacctggtgctcggcaccggcaccgtgccgttcctcccggagccgtgccgcgggctggacggcggcctcttccacaacgcgcagtacatgcagcgcaaggcggaactgctggagaagaagtcggtcacggtcgtcggcagcggtcagagcgccgccgagatctactacgagctgctctccgagatcgacgtccacggctaccagctcaactgggtcacgcgttcgccgcggttcttcccgctggagtacaccaagctgaccctggagatgacctccccggactacatcgactacttccgcgccctgcccgaggagacccgctaccggctggagaagcagcagaagggcctgttcaagggcatcaactcggagctgatcgacgccatcttcgacctgctctaccagaagaacgtcgagagcggggaccgtccggtacccacccggctgctcaccaactccagcctcaccggcgcgcggtaccaggacggcgcgtacacgctcggcttccaccaggacgagcagggcaaggacttcgagatccgcaccgagggcctggtgctggccaccggctaccactacgagccgccggcgttcctcgacccggtgcgcgagcggatccgcttcgacggccacggccgtttcgacgtcgcccgcaactacgccatcgacgtcaccggccgggggatcttcctgcagaacgcgggcgtccacacgcacagcatcaccagccccgacctgggcatgggcccgtaccgcaacgcgtccatcatccgtgagctgctgggcaccgagtactacccggtcgagaagagcatcgcgttccaggagttcgccgtatgagcatcggtacgttcaccgtccgccccctggacccgctcgaggacgccgagctgctgcaccgctgggtcaccgaccccaaggcggcgttctggatgatgcaggacgccgcgctccaggacgtcgagcgcgagtacctgcgcatcgccgcccacgagcaccaccacgcctacctgggcctgcacgacggcgaacccgccttcctgatggagaagtacgacccccggtacgtcgaactcgtcggcctgtacgaccccgagcccggcgacgtcggcatgcacttcctggtcgcccccaccgaccgccccgtccacggcttcacccgggcggtcatcaccgccgtgatggaggagctgttcgccgaccccgccacccggcgggtggtggtcgaaccggacgtcggcaacaaggccgtgcacgccctcaacgaggccgtgggcttcgtgcccgagcgggagatcgacaagccggagaagcgcgcgctgctgagcttctgcacgcgtgaacagttcgaggccgcccgaggagcatccgcatgaccctgtccgacgccgtggcgcacctgtccccccaccgctgggcgcgggccaaccgcctgctgatccgcaaggcgctcgccgagttcgcccacgagcggctcatcacgccgcaggactccggcgacggccggtacgccgtccacagcgacgacggccggacccggtacacgttcaccgctgcgcggcgcgccctggaccactggcacatcgaccccgagtcgatcacccgccaccgcgacgacgccgaacgcgagctcgcggccctggacttcttcatcgagctgcagaagtccctgggcctgagcgaggagattctgccggtgtacctggaggagatctcctccaccctctccggcacctgctacaagctcaccaagccgcaggtgcgggccggagagctgatcgacgccggcttccaggccgtcgagaccggcatgaccgaggggcacccctgcttcgtcgccaacaacgggcggctcggcttcggcgtcgacgagtaccgcgcgtacgcgccggaggcggcggccccggtcaagctggtgtggctggccgcccaccgctcgcgggccgcgttcacggcgggcgtgggcatcgagtacgagtcgttcgtacggcaggaactgggcgaggagaccgtcgagggcttccacgacgtcctgcgcgcgcaggacctcgaccccggcgactacctcctgatacccgtccacccctggcagtggtggaacaagctgtccgtcaccttcgccgccgagatcgcccgccggcacctggtgtgcctgggcgagggcgacgacgagtacctggcccagcagtccatccgcaccttcttcaaccggtcgcaccccgggaagcactacgtgaagacggccctgtccgtcctcaacatgggcttcatgcgcgggctctccgccgcctacatggaggcgaccccggcgatcaacgactggctggcgcagctcgtcgacagcgacccggtgctgcgcgcgaccgggctgtcgatcatccgggagcgggccgccgtgggctaccggcacctggagtacgagcaggccacggaccgctactcgccgtaccgcaagatgctggccgcgctgtggcgggagagcccggtgccgtccctggaggacggcgagtccctcgccaccatggcctcgctggtccatgtcgaccacgagggcgcctccgtggcggccgcgctgatcgcacgctccgggctggcccccaccgagtggctgcgccactacctgcgcgcctactacgtgccgctgctgcacagcttctacgcctacgacctggcctacatgccgcacggcgagaacgtgatcctcgtgctgcgggacggcgtggtgcggcgcgcgatctacaaggacatcgccgaggagatcgtggtgatggacgccgacgcggtgctgccgccggaggtggcccgcatccgggcggaggtgccggacgacacgaagctgctgtcgatcttcacggacgtcttcgactgcttcttccgcttcctcgccgcgaacctggccgaggagggcgtcctggacgaggacggcttctggcgcacggtcgcggaggtcacccgcgagtaccaggcgtcggtgccggaactcgccgacaagttcgcccggtacgacatgttcgccccggagttcgccctgtcctgcctgaaccgcctgcagttgcgcaacaaccggcagatggtcgacctgtcggacccctccggcgcgctccagctggtcggcaccctgaagaaccccctcgcggagttctgacccacgaaaagcttgcatgcctgcaggtcgact

SEQ ID NO: 30 - DNA, 11659 bp, synthetic construct
  misc_feature 1..11659: note = Vector Construct
  source 1..11659: mol_type = other DNA
aattaagctttgcaggaaggagatatagatatgcgctcgcacctgctcaacgacaccaccgcggagcactaccgccgctccgtgaccgagggagtcgagcgggtggcggccaaactcgccaccacagcccggccgttcaccggcgtcacggtcgacgccctcgcgccccgcatcgacgcgatcgacctcgaccggcccctgcacgacaccaccgcggtactggacgaactggaggacgtctacctccgggacgcgatctacttccaccacccgcgctacctcgcccacctcaactgcccggtcgtcatcccggccctgctcggcgaggcggtgctctccgccgtcaactcctccctggacacctgggaccagtcggcgggcggcaccctcatcgagcgcaagctcatcgactggacgaccgcccgcatcggcctcggccccgccgccgacggcgtgttcacctccggcggcacccagtccaacctccaggcgctgctgctcgcccgcgaggaggccaagaccggtgacctggcgaaactgcgcgtcttcgcctccgaggtcagccacttcagcgtgaagaagtccgcgaaactgctcggcctcgggcccgacgccgtgatctcgatcccagtcgaccacgacaagcggctgcagaccgtcgccctcgcccgcgagctggagcggtgcgtcgaggcgggcctcgtccccatggccgtcgtcgccaccgccggcaccaccgacttcggctccatcgacccgctgcccgggatcgccgaactgtgcgggcggtacggcgcctggatgcacgtcgacgccgcctacggctgcggtctgctcgcctcactgaagtaccgcgaccgcatcgccggcatcgagcacgccgactccgtcaccgtcgactaccacaagtccttcttccagcccgtcagttcgtcggccgtactggtccgcgacaccgccacgctgcggcacgccacctaccacgccgagtacctcaacccgcgccgcatggtgatcgagcgcatccccaaccaggtcgacaagtccctgcagaccacccgccgcttcgacgccctgaaactgtggatgacgctgcgcgtgatgggtgccgacggcatcggcggcctcttcgacgaggtgtgcgacctggccgcggagggctggaagctgctcgccgccgacccgcgcttcgacgtcgtggtcgcgcccagcctctccaccctcgtcttccgctacatcccggcggccgtcaccgaccccgccgagatcgaccgcgccaacctctacgcccgcaaggccctcttcgcctccggcgacgcggtcgtcgccggcaccaaggtggccgggcgccactacctgaagttcaccctgctgaaccccgagaccacgacggacgacatctccgccgtcctcgatctgatcgccggccacgccgagcagtacctgggagagtcccttgaccgcgcttgctgaagccccggaaaccacctacgacttcgtgggcgtcgggctgggccccttcaacctcggcctcgcctgcctcaccgagccgatatccgaactgaacggcgtcttcctggagtccaagccggacttcgcctggcacgccgggatgttcctcgacggcgcccacctccagacccccttcatgtcggacctggtcaccctcgccgaccccacctccccctactccttcctcaactacctgaaggagaagggccggctgtactcgttctacatacgcgagaacttctacccgctgcgggtcgagtacgacgactactgccgctgggccgccgccaagctgagcagcatccgcttcggcacgacggtcaccgaggtgacctacgacgaggccgccgagctgtacgcggtcgcgacggcggccggtgagacgtaccgcgcccggcacctggtgctcggcaccggcaccgtgccgttcctcccggagccgtgccgcgggctggacggcggcctcttccacaacgcgcagtacatgcagcgcaaggcggaactgctggagaagaagtcggtcacggtcgtcggcagcggtcagagcgccgccgagatctactacgagctgctctccgagatcgacgtccacggctaccagctcaactgggtcacgcgttcgccgcggttcttcccgctggagtacaccaagctgaccctggagatgacctccccggactacatcgactacttccgcgccctgcccgaggagacccgctaccggctggagaagcagcagaagggcctgttcaagggcatcaactcggagctgatcgacgccatcttcgacctgctctaccagaagaacgtcgagagcggggaccgtccggtacccacccggctgctcaccaactccagcctcaccggcgcgcggtaccaggacggcgcgtacacgctcggcttccaccaggacgagcagggcaaggacttcgagatccgcaccgagggcctggtgctggccaccggctaccactacgagccgccggcgttcctcgacccggtgcgcgagcggatccgcttcgacggccacggccgtttcgacgtcgcccgcaactacgccatcgacgtcaccggccgggggatcttcctgcagaacgcgggcgtccacacgcacagcatcaccagccccgacctgggcatgggcccgtaccgcaacgcgtccatcatccgtgagctgctgggcaccgagtactacccggtcgagaagagcatcgcgttccaggagttcgccgtatgagcatcggtacgttcaccgtccgccccctggacccgctcgaggacgccgagctgctgcaccgctgggtcaccgaccccaaggcggcgttctggatgatgcaggacgccgcgctccaggacgtcgagcgcgagtacctgcgcatcgccgcccacgagcaccaccacgcctacctgggcctgcacgacggcgaacccgccttcctgatggagaagtacgacccccggtacgtcgaactcgtcggcctgtacgaccccgagcccggcgacgtcggcatgcacttcctggtcgcccccaccgaccgccccgtccacggcttcacccgggcggtcatcaccgccgtgatggaggagctgttcgccgaccccgccacccggcgggtggtggtcgaaccggacgtcggcaacaaggccgtgcacgccctcaacgaggccgtgggcttcgtgcccgagcgggagatcgacaagccggagaagcgcgcgctgctgagcttctgcacgcgtgaacagttcgaggccgcccgaggagcatccgcatgaccctgtccgacgccgtggcgcacctgtccccccaccgctgggcgcgggccaaccgcctgctgatccgcaaggcgctcgccgagttcgcccacgagcggctcatcacgccgcaggactccggcgacggccggtacgccgtccacagcgacgacggccggacccggtacacgttcaccgctgcgcggcgcgccctggaccactggcacatcgaccccgagtcgatcacccgccaccgcgacgacgccgaacgcgagctcgcggccctggacttcttcatcgagctgcagaagtccctgggcctgagcgaggagattctgccggtgtacctggaggagatctcctccaccctctccggcacctgctacaagctcaccaagccgcaggtgcgggccggagagctgatcgacgccggcttccaggccgtcgagaccggcatgaccgaggggcacccctgcttcgtcgccaacaacgggcggctcggcttcggcgtcgacgagtaccgcgcgtacgcgccggaggcggcggccccggtcaagctggtgtggctggccgcccaccgctcgcgggccgcgttcacggcgggcgtgggcatcgagtacgagtcgttcgtacggcaggaactgggcgaggagaccgtcgagggcttccacgacgtcctgcgcgcgcaggacctcgaccccggcgactacctcctgatacccgtccacccctggcagtggtggaacaagctgtccgtcaccttcgccgccgagatcgcccgccggcacctggtgtgcctgggcgagggcgacgacgagtacctggcccagcagtccatccgcaccttcttcaaccggtcgcaccccgggaagcactacgtgaagacggccctgtccgtcctcaacatgggcttcatgcgcgggctctccgccgcctacatggaggcgaccccggcgatcaacgactggctggcgcagctcgtcgacagcgacccggtgctgcgcgcgaccgggctgtcgatcatccgggagcgggccgccgtgggctaccggcacctggagtacgagcaggccacggaccgctactcgccgtaccgcaagatgctggccgcgctgtggcgggagagcccggtgccgtccctggaggacggcgagtccctcgccaccatggcctcgctggtccatgtcgaccacgagggcgcctccgtggcggccgcgctgatcgcacgctccgggctggcccccaccgagtggctgcgccactacctgcgcgcctactacgtgccgctgctgcacagcttctacgcctacgacctggcctacatgccgcacggcgagaacgtgatcctcgtgctgcgggacggcgtggtgcggcgcgcgatctacaaggacatcgccgaggagatcgtggtgatggacgccgacgcggtgctgccgccggaggtggcccgcatccgggcggaggtgccggacgacacgaagctgctgtcgatcttcacggacgtcttcgactgcttcttccgcttcctcgccgcgaacctggccgaggagggcgtcctggacgaggacggcttctggcgcacggtcgcggaggtcacccgcgagtaccaggcgtcggtgccggaactcgccgacaagttcgcccggtacgacatgttcgccccggagttcgccctgtcctgcctgaaccgcctgcagttgcgcaacaaccggcagatggtcgacctgtcggacccctccggcgcgctccagctggtcggcaccctgaagaaccccctcgcggagttctgacccacgaaaagcttgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 31 - DNA, 1293 bp, Erwinia amylovora
  source 1..1293: mol_type = genomic DNA
atgaataacaccatctatgattttattggtattggcattggaccattcaatctgggccttgcctgtctgagcgaaccggttgaagggttgaacggcgtttttctcgatcagaaccccggctttgactggcataccggcatgatgctggaaagcgcccatttgcaaacgccttttatggccgatctggtgacgctggccgatccgaccagcccttacagcctgctcaattttatgaagcagaagggaaaactttactctttttacatccgcgaagattttttcctgatgagaaaagagtacaaccagtattgccaatgggccgctgaacggctcggcaacctgcgctggaacacccgggttgaatacgtcagctacgatgacaatctgcaatgctatcgtgtccgttcgacagataccgtcagcggtaaacagcaggaatggctggcgcatcgtctggtactgggcaccggccccagcgcctggagtcccgcctgcagccaaccgtatcgcgagcgcttcgtccactccagcgaatatctgctcaacaaagaaaaattgcagaaaaaacgctccatcaccgtactgggcagcggccagagcgcggccgaaatctattacgatctgctgacggatatcgatcgtttcggctatcagctgaactggataactcgcgcgccgcgcttctacccgcttgagtacacgaaactgacgctggaaatgacctcgccagaatggattgactattttcacagtctgccggcggcgaaacgcgacgagcttaacgccagccagaaaaatctctataaaggcatcaacagcagcctgattaatgcgatttacgacctgttgtacgttaagcagctggacggaaagctcgacgtcaatctctttacccattcggagctgaccgacatgcgctggctggcggaaggtgagtttgagctcaagctgcatcagcaggaacaggatcgtgcctacagccgccgtactgagggattagtgatggccacgggctatcactatcaaccaccggcgtttgttgaaggcatccagcagcggatccaatgggatgagaaagaccgttatgacgtacaacgtaattacagcatcgatcgccataaccaggtcttcgtgcaaaatgccgagctgcatacgcacggctttgtcacgcctgatttaggcatggcctgctaccgcaattccgtgttactgcgcgaaatcaccggccgggaagtctaccccgtcgaacgccagatcgctttccagactttccccgcacaatcggaaatgtga

SEQ ID NO: 32 - DNA, 2355 bp, Erwinia amylovora
  source 1..2355: mol_type = genomic DNA
atgccaaattccactctttacagtacgggccgtcccgcgggccggttcaccctccgcccaatgcgccatgaagacgccgcaatggtccaccgttgggtcacacaagagtatgcccgcttctgggggatgcaggacaacagcatcgaacaggttgcggccttttatcaccagctgacggctcataacccgcacgccgcgctgatcggttgctgcaacgaccagccggtgttcctgatggagttctacaaggccagtgaggatgacatcggcaagttctatgccgcgcagccgggcgattacggtatgcatctgctgatagcgcctgccactcacccggtgcagcagttcagctggcaggttttctcgacggtaatcgactttatgttcagcctgccggaggttaagcgggtagtggtggagccggacgagcgtaatacaaaaattcatcgactgaacaagcgcgcgggcttttgctaccagcacaccatcgatatgggccataaaaccgcctggctggccttctgccagcgcgaaaattatcaacaggctttactgaaggaatcgctgaatatgaatgatacccatctgctgcaagccggcacctggctgaccggagataactgggctgaggccaaccgtctgctgatccgcaaagctatcgccgagttcgctcatgagaaaatcgtaacgcctgctgaatgcgctcacggccgctactctctggccgtgccgggcagcgaaacagaataccagtttaccgccagccgcctcgcgctggatcactgggagatagacgccgcttcgctgactaaacaggaaaacggtcatcccctggcgctggacgccttacaatttattacggaattcaatgaggtaatcggtattccacaggctctgctggcaacctatatggaagagatcagcagcacgttgtgcagcagcgttttcaaactgcagaagaataacccggacagccgggcactggtcaacgcagactttcagaccgttgaatcctcaatgaccgaagggcacccgtgcttcgtggcgaacaacgggcgcattggctttgatgcgcgtgactatctggcttacgcgcctgaagctgccacgccggttaatcttatctgggttgccgtgcaccggcgcaacgcccacttctccagcctgagtgacctgcaatacgagcgcctgatgcgtgaagaactggggcagtcaacggttgaacagtttaatgcacagctgacggagaaaggacttacccacgctgattacctctttatgccggttcacccctggcagtggcagaacaagctgctgacggtatttgcggcagatatcgccaataacgatattgtctggcttggcgtcggcgacgatcaatatcaggcgcagcagtctattcggacctttttcaaccgcagccatcctaacaagcgctatgtcaaaacagcactgtcagtgctgaatatgggctttatgcgcggtctttctccctattatatggcgaccacgccggcgattaatgaatggctgcaagatctggtggccggggatgaatggctacagcgctgtgatttccgcattctgcgcgaagtggcggcagtgggttaccataaccgccactatgaaaaagccatcaagggtgattccgcctacaagaaaatgtttgccgcgctgtggcgtgataatccggttgccgagctcaaacccggccagcgcctgatgaccatggcctcattcctgcacgttgatcatcaccagaaagccctgctgccagcgctgatcgccgattccgggctggccgctgaacgctgggtggaacgctatctgagctgctacctcagtccgctgctgcactgcttctaccagcacgatctggtgtttatgccgcacggcgaaaacctgatcctgctgctggagaataacgtcccggtcagcgcctatatgaaagacatcggcgaagaaattgcggtgatgaacccggatgccgtgctgccggaaaaagtacagcgcctggcggtggacgtgccggaaaacctcaagctgctgtcggtctttaccgacgtgttcgactgcattttccgctttatcagcgctattcttcaccagtccgccactctgccggaagaacaattctggcaggcggtcgcgcgctgcgtgaaggagtaccagcaggcccatccgcatctggccagcaagttttcccgttacgatatgttcgcgccggaattcacccgctcctgcctcaaccgactacagctggccaataaccagcaaatgatcaatctgtcagacccggctgaaaatctgaaatttgccggtacgctggtcaacccgatcgcccgctggcgttaa

SEQ ID NO: 33 - protein, 430 aa, Erwinia amylovora
MNNTIYDFIGIGIGPFNLGLACLSEPVEGLNGVFLDQNPGFDWHTGMMLESAHLQTPFMADLVTLADPTSPYSLLNFMKQKGKLYSFYIREDFFLMRKEYNQYCQWAAERLGNLRWNTRVEYVSYDDNLQCYRVRSTDTVSGKQQEWLAHRLVLGTGPSAWSPACSQPYRERFVHSSEYLLNKEKLQKKRSITVLGSGQSAAEIYYDLLTDIDRFGYQLNWITRAPRFYPLEYTKLTLEMTSPEWIDYFHSLPAAKRDELNASQKNLYKGINSSLINAIYDLLYVKQLDGKLDVNLFTHSELTDMRWLAEGEFELKLHQQEQDRAYSRRTEGLVMATGYHYQPPAFVEGIQQRIQWDEKDRYDVQRNYSIDRHNQVFVQNAELHTHGFVTPDLGMACYRNSVLLREITGREVYPVERQIAFQTFPAQSEM

SEQ ID NO: 34 - protein, 784 aa, Erwinia amylovora
MPNSTLYSTGRPAGRFTLRPMRHEDAAMVHRWVTQEYARFWGMQDNSIEQVAAFYHQLTAHNPHAALIGCCNDQPVFLMEFYKASEDDIGKFYAAQPGDYGMHLLIAPATHPVQQFSWQVFSTVIDFMFSLPEVKRVVVEPDERNTKIHRLNKRAGFCYQHTIDMGHKTAWLAFCQRENYQQALLKESLNMNDTHLLQAGTWLTGDNWAEANRLLIRKAIAEFAHEKIVTPAECAHGRYSLAVPGSETEYQFTASRLALDHWEIDAASLTKQENGHPLALDALQFITEFNEVIGIPQALLATYMEEISSTLCSSVFKLQKNNPDSRALVNADFQTVESSMTEGHPCFVANNGRIGFDARDYLAYAPEAATPVNLIWVAVHRRNAHFSSLSDLQYERLMREELGQSTVEQFNAQLTEKGLTHADYLFMPVHPWQWQNKLLTVFAADIANNDIVWLGVGDDQYQAQQSIRTFFNRSHPNKRYVKTALSVLNMGFMRGLSPYYMATTPAINEWLQDLVAGDEWLQRCDFRILREVAAVGYHNRHYEKAIKGDSAYKKMFAALWRDNPVAELKPGQRLMTMASFLHVDHHQKALLPALIADSGLAAERWVERYLSCYLSPLLHCFYQHDLVFMPHGENLILLLENNVPVSAYMKDIGEEIAVMNPDAVLPEKVQRLAVDVPENLKLLSVFTDVFDCIFRFISAILHQSATLPEEQFWQAVARCVKEYQQAHPHLASKFSRYDMFAPEFTRSCLNRLQLANNQQMINLSDPAENLKFAGTLVNPIARWR

SEQ ID NO: 35 - DNA, 3830 bp, synthetic construct
  misc_feature 1..3830: note = Vector Construct
  source 1..3830: mol_type = other DNA
aagcttagacgtgattgctctgattgtgcattacggccgtgaacaggtacgtggcccggccgtcacgacggcacctttgtgagatctccgttatgaataacaccatctatgattttattggtattggcattggaccattcaatctgggccttgcctgtctgagcgaaccggttgaagggttgaacggcgtttttctcgatcagaaccccggctttgactggcataccggcatgatgctggaaagcgcccatttgcaaacgccttttatggccgatctggtgacgctggccgatccgaccagcccttacagcctgctcaattttatgaagcagaagggaaaactttactctttttacatccgcgaagattttttcctgatgagaaaagagtacaaccagtattgccaatgggccgctgaacggctcggcaacctgcgctggaacacccgggttgaatacgtcagctacgatgacaatctgcaatgctatcgtgtccgttcgacagataccgtcagcggtaaacagcaggaatggctggcgcatcgtctggtactgggcaccggccccagcgcctggagtcccgcctgcagccaaccgtatcgcgagcgcttcgtccactccagcgaatatctgctcaacaaagaaaaattgcagaaaaaacgctccatcaccgtactgggcagcggccagagcgcggccgaaatctattacgatctgctgacggatatcgatcgtttcggctatcagctgaactggataactcgcgcgccgcgcttctacccgcttgagtacacgaaactgacgctggaaatgacctcgccagaatggattgactattttcacagtctgccggcggcgaaacgcgacgagcttaacgccagccagaaaaatctctataaaggcatcaacagcagcctgattaatgcgatttacgacctgttgtacgttaagcagctggacggaaagctcgacgtcaatctctttacccattcggagctgaccgacatgcgctggctggcggaaggtgagtttgagctcaagctgcatcagcaggaacaggatcgtgcctacagccgccgtactgagggattagtgatggccacgggctatcactatcaaccaccggcgtttgttgaaggcatccagcagcggatccaatgggatgagaaagaccgttatgacgtacaacgtaattacagcatcgatcgccataaccaggtcttcgtgcaaaatgccgagctgcatacgcacggctttgtcacgcctgatttaggcatggcctgctaccgcaattccgtgttactgcgcgaaatcaccggccgggaagtctaccccgtcgaacgccagatcgctttccagactttccccgcacaatcggaaatgtgatatgccaaattccactctttacagtacgggccgtcccgcgggccggttcaccctccgcccaatgcgccatgaagacgccgcaatggtccaccgttgggtcacacaagagtatgcccgcttctgggggatgcaggacaacagcatcgaacaggttgcggccttttatcaccagctgacggctcataacccgcacgccgcgctgatcggttgctgcaacgaccagccggtgttcctgatggagttctacaaggccagtgaggatgacatcggcaagttctatgccgcgcagccgggcgattacggtatgcatctgctgatagcgcctgccactcacccggtgcagcagttcagctggcaggttttctcgacggtaatcgactttatgttcagcctgccggaggttaagcgggtagtggtggagccggacgagcgtaatacaaaaattcatcgactgaacaagcgcgcgggcttttgctaccagcacaccatcgatatgggccataaaaccgcctggctggccttctgccagcgcgaaaattatcaacaggctttactgaaggaatcgctgaatatgaatgatacccatctgctgcaagccggcacctggctgaccggagataactgggctgaggccaaccgtctgctgatccgcaaagctatcgccgagttcgctcatgagaaaatcgtaacgcctgctgaatgcgctcacggccgctactctctggccgtgccgggcagcgaaacagaataccagtttaccgccagccgcctcgcgctggatcactgggagatagacgccgcttcgctgactaaacaggaaaacggtcatcccctggcgctggacgccttacaatttattacggaattcaatgaggtaatcggtattccacaggctctgctggcaacctatatggaagagatcagcagcacgttgtgcagcagcgttttcaaactgcagaagaataacccggacagccgggcactggtcaacgcagactttcagaccgttgaatcctcaatgaccgaagggcacccgtgcttcgtggcgaacaacgggcgcattggctttgatgcgcgtgactatctggcttacgcgcctgaagctgccacgccggttaatcttatctgggttgccgtgcaccggcgcaacgcccacttctccagcctgagtgacctgcaatacgagcgcctgatgcgtgaagaactggggcagtcaacggttgaacagtttaatgcacagctgacggagaaaggacttacccacgctgattacctctttatgccggttcacccctggcagtggcagaacaagctgctgacggtatttgcggcagatatcgccaataacgatattgtctggcttggcgtcggcgacgatcaatatcaggcgcagcagtctattcggacctttttcaaccgcagccatcctaacaagcgctatgtcaaaacagcactgtcagtgctgaatatgggctttatgcgcggtctttctccctattatatggcgaccacgccggcgattaatgaatggctgcaagatctggtggccggggatgaatggctacagcgctgtgatttccgcattctgcgcgaagtggcggcagtgggttaccataaccgccactatgaaaaagccatcaagggtgattccgcctacaagaaaatgtttgccgcgctgtggcgtgataatccggttgccgagctcaaacccggccagcgcctgatgaccatggcctcattcctgcacgttgatcatcaccagaaagccctgctgccagcgctgatcgccgattccgggctggccgctgaacgctgggtggaacgctatctgagctgctacctcagtccgctgctgcactgcttctaccagcacgatctggtgtttatgccgcacggcgaaaacctgatcctgctgctggagaataacgtcccggtcagcgcctatatgaaagacatcggcgaagaaattgcggtgatgaacccggatgccgtgctgccggaaaaagtacagcgcctggcggtggacgtgccggaaaacctcaagctgctgtcggtctttaccgacgtgttcgactgcattttccgctttatcagcgctattcttcaccagtccgccactctgccggaagaacaattctggcaggcggtcgcgcgctgcgtgaaggagtaccagcaggcccatccgcatctggccagcaagttttcccgttacgatatgttcgcgccggaattcacccgctcctgcctcaaccgactacagctggccaataaccagcaaatgatcaatctgtcagacccggctgaaaatctgaaatttgccggtacgctggtcaacccgatcgcccgctggcgttaacgtcctccttcttctgggtaaaaaaaagcacggccgcttaaacggccgtgctttttattcaggtaaaccgaatcgcgggttagaagctt

SEQ ID NO: 36 - DNA, 10415 bp, synthetic construct
  misc_feature 1..10415: note = Vector Construct
  source 1..10415: mol_type = other DNA
aattaagacgtgattgctctgattgtgcattacggccgtgaacaggtacgtggcccggccgtcacgacggcacctttgtgagatctccgttatgaataacaccatctatgattttattggtattggcattggaccattcaatctgggccttgcctgtctgagcgaaccggttgaagggttgaacggcgtttttctcgatcagaaccccggctttgactggcataccggcatgatgctggaaagcgcccatttgcaaacgccttttatggccgatctggtgacgctggccgatccgaccagcccttacagcctgctcaattttatgaagcagaagggaaaactttactctttttacatccgcgaagattttttcctgatgagaaaagagtacaaccagtattgccaatgggccgctgaacggctcggcaacctgcgctggaacacccgggttgaatacgtcagctacgatgacaatctgcaatgctatcgtgtccgttcgacagataccgtcagcggtaaacagcaggaatggctggcgcatcgtctggtactgggcaccggccccagcgcctggagtcccgcctgcagccaaccgtatcgcgagcgcttcgtccactccagcgaatatctgctcaacaaagaaaaattgcagaaaaaacgctccatcaccgtactgggcagcggccagagcgcggccgaaatctattacgatctgctgacggatatcgatcgtttcggctatcagctgaactggataactcgcgcgccgcgcttctacccgcttgagtacacgaaactgacgctggaaatgacctcgccagaatggattgactattttcacagtctgccggcggcgaaacgcgacgagcttaacgccagccagaaaaatctctataaaggcatcaacagcagcctgattaatgcgatttacgacctgttgtacgttaagcagctggacggaaagctcgacgtcaatctctttacccattcggagctgaccgacatgcgctggctggcggaaggtgagtttgagctcaagctgcatcagcaggaacaggatcgtgcctacagccgccgtactgagggattagtgatggccacgggctatcactatcaaccaccggcgtttgttgaaggcatccagcagcggatccaatgggatgagaaagaccgttatgacgtacaacgtaattacagcatcgatcgccataaccaggtcttcgtgcaaaatgccgagctgcatacgcacggctttgtcacgcctgatttaggcatggcctgctaccgcaattccgtgttactgcgcgaaatcaccggccgggaagtctaccccgtcgaacgccagatcgctttccagactttccccgcacaatcggaaatgtgatatgccaaattccactctttacagtacgggccgtcccgcgggccggttcaccctccgcccaatgcgccatgaagacgccgcaatggtccaccgttgggtcacacaagagtatgcccgcttctgggggatgcaggacaacagcatcgaacaggttgcggccttttatcaccagctgacggctcataacccgcacgccgcgctgatcggttgctgcaacgaccagccggtgttcctgatggagttctacaaggccagtgaggatgacatcggcaagttctatgccgcgcagccgggcgattacggtatgcatctgctgatagcgcctgccactcacccggtgcagcagttcagctggcaggttttctcgacggtaatcgactttatgttcagcctgccggaggttaagcgggtagtggtggagccggacgagcgtaatacaaaaattcatcgactgaacaagcgcgcgggcttttgctaccagcacaccatcgatatgggccataaaaccgcctggctggccttctgccagcgcgaaaattatcaacaggctttactgaaggaatcgctgaatatgaatgatacccatctgctgcaagccggcacctggctgaccggagataactgggctgaggccaaccgtctgctgatccgcaaagctatcgccgagttcgctcatgagaaaatcgtaacgcctgctgaatgcgctcacggccgctactctctggccgtgccgggcagcgaaacagaataccagtttaccgccagccgcctcgcgctggatcactgggagatagacgccgcttcgctgactaaacaggaaaacggtcatcccctggcgctggacgccttacaatttattacggaattcaatgaggtaatcggtattccacaggctctgctggcaacctatatggaagagatcagcagcacgttgtgcagcagcgttttcaaactgcagaagaataacccggacagccgggcactggtcaacgcagactttcagaccgttgaatcctcaatgaccgaagggcacccgtgcttcgtggcgaacaacgggcgcattggctttgatgcgcgtgactatctggcttacgcgcctgaagctgccacgccggttaatcttatctgggttgccgtgcaccggcgcaacgcccacttctccagcctgagtgacctgcaatacgagcgcctgatgcgtgaagaactggggcagtcaacggttgaacagtttaatgcacagctgacggagaaaggacttacccacgctgattacctctttatgccggttcacccctggcagtggcagaacaagctgctgacggtatttgcggcagatatcgccaataacgatattgtctggcttggcgtcggcgacgatcaatatcaggcgcagcagtctattcggacctttttcaaccgcagccatcctaacaagcgctatgtcaaaacagcactgtcagtgctgaatatgggctttatgcgcggtctttctccctattatatggcgaccacgccggcgattaatgaatggctgcaagatctggtggccggggatgaatggctacagcgctgtgatttccgcattctgcgcgaagtggcggcagtgggttaccataaccgccactatgaaaaagccatcaagggtgattccgcctacaagaaaatgtttgccgcgctgtggcgtgataatccggttgccgagctcaaacccggccagcgcctgatgaccatggcctcattcctgcacgttgatcatcaccagaaagccctgctgccagcgctgatcgccgattccgggctggccgctgaacgctgggtggaacgctatctgagctgctacctcagtccgctgctgcactgcttctaccagcacgatctggtgtttatgccgcacggcgaaaacctgatcctgctgctggagaataacgtcccggtcagcgcctatatgaaagacatcggcgaagaaattgcggtgatgaacccggatgccgtgctgccggaaaaagtacagcgcctggcggtggacgtgccggaaaacctcaagctgctgtcggtctttaccgacgtgttcgactgcattttccgctttatcagcgctattcttcaccagtccgccactctgccggaagaacaattctggcaggcggtcgcgcgctgcgtgaaggagtaccagcaggcccatccgcatctggccagcaagttttcccgttacgatatgttcgcgccggaattcacccgctcctgcctcaaccgactacagctggccaataaccagcaaatgatcaatctgtcagacccggctgaaaatctgaaatttgccggtacgctggtcaacccgatcgcccgctggcgttaacgtcctccttcttctgggtaaaaaaaagcacggccgcttaaacggccgtgctttttattcaggtaaaccgaatcgcgggttagtgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 37 - DNA, 1293 bp, Pantoea agglomerans
  source 1..1293: mol_type = genomic DNA
atgaataattccgtttacgattttattggtatcggtatcggccctttcaacctgagtctggcctgtctgagtgagccggttgagggactgaacggtcttttcctggatgagaacccaggctttgactggcacaccggcatgatgctggaaaatgcccatctgcagacgccgtttatggccgatctggtgacactcgccgatccgaccagcccttacagcctgcttaactacatgaaggaaaaggggaaattatactctttttatatccgtgaagacttcttcctgatgcgcaaagagtataatcagtactgccagtgggccagttcacggctctctaatctgcgctggaacagccacgttgagtatgtcagctacgatgaggcggctggcgtctatcaactccaggtgaccgacacccggaatggtcagaaacagaactatctggcacgacatctgatcctcggcaccgggcctaaagcctggatgccggagtgcagccagccgcaccgccagcgcctgacgcactcaagccactatctggtcaacaaagcggaactgcagcagaagcgttccatcaccgtgctgggcagcggccagagcgcggcagaaatctactacgacctgctgaccgatatcgaccgctttggctatcagctcaactggatcacccgcgcgccgcgcttctatccgctggagtacaccaagctcacgctggagatgacttcgccggagtgggtcgactatttccacgcgctgcccgccagcacccgcgacgaactgaatgcgcgccacaagaatctctacaaaggcatcaacagcagcctgattaacgacatttatgacctgatgtatgtcaaacagctcgacggcgatctgaacgttaacctgtttactcactcggcgctgaccgctatgcgctggctgccacagggagagttcgagctgacgctgcatcaggaggagcaggattgcaccttcacccgtcgcaccgaaggactggtgatggcgaccggctatcactaccagccaccgatgtttttagagggcatcaacgcccgcctgcgctgggatgacaaaggccgctacgacgtgcagcgcaactacagcatcgattatcacaatcagatcttcgtgcagaacgccgaactgcacacccacggttttgttacgccggatctcggcatggcctgttaccgcaactcggtgctgctgcgcgaactgaccggacgcgaggtctacccgatcgagcgtcagattgccttccagaccttcccggctaaatcggagcactga

SEQ ID NO: 38 - DNA, 2355 bp, Pantoea agglomerans
  source 1..2355: mol_type = genomic DNA
atgagttcacaaacactgttcagcgcagcccgccccgcaggccagttcaccctgaggcccatgctcgccagcgatgcgccgctgattcatcgctgggtcacccgcgattacgcccgcttctggggcatgcagcagcagagcctgaatcaggtcgctgagttctaccagacgctgaccaaagccgatccccatgctgcgctgattggttgctgcaacgaccagccggtctgcctgattgagtgctatcgcgcctgcgacgatgaggtcggcaaattttatcctgccgcacccgatgattacggcatgcatattctgattgcccccgccagccagccgatcagagccttcagctggcaggtcttcacgctggtgatggactacatgttcagccgccccgaggtgcgccgcgtggtggtagagccagatgtgcgcaatgagaagattcacgtgctgaataagcgtgccgggttccgctatcagcacaccatcgacatgggtcataaaaccgcctggctggccttttgccagcgggaagattatcaacacgcgttactgcaggagtcgcagactatgaacaccccctcttcgctgatgaacggtacacatctgacgggcgaccaatgggcgcaggcaaaccgcatgctgatccggaaagcgattgccgaattcgcgcatgagaaaatcattacgccacaggagtcgggcgacggcagctatcagcttgcggtgccaggcggtgaagcgagctaccatttccgggcgtcgcggctggcgctggatcactgggaaattgacgccggctcactgcgcaagcaggagaacggtcgtgatctgccgctggatgcgctgaagtttatcgtggagtttaacggggagatcggtattacacaggcactgctggcgacctacatggaagagatcagcagcacgctgtgcagtagcgtttataagctgcagaagaataatcctgacagccaggcgctggtctcagccgatttccagacggtggaagcggcgatgaccgaaggccatccctgctttgttgccaacaacgggcgtattggcttcgatgcccatgattatctggcgtatgcgcccgaggccgccgcgcccgttcacctcatctgggtcgcggttcatcgccgcaacgcccatttctccagcctcagcgagctgagctatgagcagttgatgcgtgatgaactgggcgacaccaccctggagcagtttactgcgcagctggtgaataaagggctggcggttgaggagtatatcctgatgccggtgcatccgtggcagtggcagaacaaactgctgaccgtatttgcggcggacatcgccagtcacgatattgtttatctcggcaccggagacgatgcttaccaggcgcagcagtcgatccgcaccttctttaaccgcagccagcccacgaagcgctacgtgaaaaccgccctgtcggtgctgaacatgggctttatgcgcggactgtccccctactatatggcgacaacgcctgcgataaacggctggctggaacaactggttgctggcgatgcgtggctgaatcgctgtgatttccgcatcctgcgcgaagtcgcggcggtcggctatcacaaccgctactatgagcaggcgatcaaaggcgactccgcctataaaaagatgttcgctgcgctgtggcgggataatcctgcggccagcctgcagcccggccagcgtctgatgaccatggcctctttcctgcacgtcgatcaccatcagcaggcactgctgccagcgctgatagcggattctggtttgcccgcgaagcagtggatagaacgctacctgacctgctatctcagcccgctgctgcactgcttctatcagcatgacctggtgtttatgccgcacggtgaaaacctcatcatgctgctggagaacaacgtgccggtcagtgcatacatgaaggatatcggcgaagagatcgccgtgatgaatccggatgcggtgctgccggaaggcgtgcagcgtctggcggtggatgtgccggagaacctgaagctgctctcgatcttcaccgatgtgtttgactgcatcttccgctttatcagcgcgattctgcatcagtcaggaacgctgccggaggaagaattctggcaggcggtggcgcagtgcgtgaaggattaccagcaggcgcacccggagcatgccactaagttcgcgcgctatgacatgttcgcgccggagtttacccgctcctgtctgaaccgcctgcagctggcgaataaccagcagatgataaacctcgccgatcccgctgagaatctcaagttcgccggaacgctgaataacccgattgcccgctggcgataa

SEQ ID NO: 39 - protein, 430 aa, Pantoea agglomerans
MNNSVYDFIGIGIGPFNLSLACLSEPVEGLNGLFLDENPGFDWHTGMMLENAHLQTPFMADLVTLADPTSPYSLLNYMKEKGKLYSFYIREDFFLMRKEYNQYCQWASSRLSNLRWNSHVEYVSYDEAAGVYQLQVTDTRNGQKQNYLARHLILGTGPKAWMPECSQPHRQRLTHSSHYLVNKAELQQKRSITVLGSGQSAAEIYYDLLTDIDRFGYQLNWITRAPRFYPLEYTKLTLEMTSPEWVDYFHALPASTRDELNARHKNLYKGINSSLINDIYDLMYVKQLDGDLNVNLFTHSALTAMRWLPQGEFELTLHQEEQDCTFTRRTEGLVMATGYHYQPPMFLEGINARLRWDDKGRYDVQRNYSIDYHNQIFVQNAELHTHGFVTPDLGMACYRNSVLLRELTGREVYPIERQIAFQTFPAKSEH

SEQ ID NO: 40 - protein, 784 aa, Pantoea agglomerans
MSSQTLFSAARPAGQFTLRPMLASDAPLIHRWVTRDYARFWGMQQQSLNQVAEFYQTLTKADPHAALIGCCNDQPVCLIECYRACDDEVGKFYPAAPDDYGMHILIAPASQPIRAFSWQVFTLVMDYMFSRPEVRRVVVEPDVRNEKIHVLNKRAGFRYQHTIDMGHKTAWLAFCQREDYQHALLQESQTMNTPSSLMNGTHLTGDQWAQANRMLIRKAIAEFAHEKIITPQESGDGSYQLAVPGGEASYHFRASRLALDHWEIDAGSLRKQENGRDLPLDALKFIVEFNGEIGITQALLATYMEEISSTLCSSVYKLQKNNPDSQALVSADFQTVEAAMTEGHPCFVANNGRIGFDAHDYLAYAPEAAAPVHLIWVAVHRRNAHFSSLSELSYEQLMRDELGDTTLEQFTAQLVNKGLAVEEYILMPVHPWQWQNKLLTVFAADIASHDIVYLGTGDDAYQAQQSIRTFFNRSQPTKRYVKTALSVLNMGFMRGLSPYYMATTPAINGWLEQLVAGDAWLNRCDFRILREVAAVGYHNRYYEQAIKGDSAYKKMFAALWRDNPAASLQPGQRLMTMASFLHVDHHQQALLPALIADSGLPAKQWIERYLTCYLSPLLHCFYQHDLVFMPHGENLIMLLENNVPVSAYMKDIGEEIAVMNPDAVLPEGVQRLAVDVPENLKLLSIFTDVFDCIFRFISAILHQSGTLPEEEFWQAVAQCVKDYQQAHPEHATKFARYDMFAPEFTRSCLNRLQLANNQQMINLADPAENLKFAGTLNNPIARWR

SEQ ID NO: 41 - DNA, 3842 bp, synthetic construct
  misc_feature 1..3842: note = Vector Construct
  source 1..3842: mol_type = other DNA
aagcttgagcaggtacgtgcgtctgcgctgaacgcggcgaaacagtgagagctctgaaatgaataattccgtttacgattttattggtatcggtatcggccctttcaacctgagtctggcctgtctgagtgagccggttgagggactgaacggtcttttcctggatgagaacccaggctttgactggcacaccggcatgatgctggaaaatgcccatctgcagacgccgtttatggccgatctggtgacactcgccgatccgaccagcccttacagcctgcttaactacatgaaggaaaaggggaaattatactctttttatatccgtgaagacttcttcctgatgcgcaaagagtataatcagtactgccagtgggccagttcacggctctctaatctgcgctggaacagccacgttgagtatgtcagctacgatgaggcggctggcgtctatcaactccaggtgaccgacacccggaatggtcagaaacagaactatctggcacgacatctgatcctcggcaccgggcctaaagcctggatgccggagtgcagccagccgcaccgccagcgcctgacgcactcaagccactatctggtcaacaaagcggaactgcagcagaagcgttccatcaccgtgctgggcagcggccagagcgcggcagaaatctactacgacctgctgaccgatatcgaccgctttggctatcagctcaactggatcacccgcgcgccgcgcttctatccgctggagtacaccaagctcacgctggagatgacttcgccggagtgggtcgactatttccacgcgctgcccgccagcacccgcgacgaactgaatgcgcgccacaagaatctctacaaaggcatcaacagcagcctgattaacgacatttatgacctgatgtatgtcaaacagctcgacggcgatctgaacgttaacctgtttactcactcggcgctgaccgctatgcgctggctgccacagggagagttcgagctgacgctgcatcaggaggagcaggattgcaccttcacccgtcgcaccgaaggactggtgatggcgaccggctatcactaccagccaccgatgtttttagagggcatcaacgcccgcctgcgctgggatgacaaaggccgctacgacgtgcagcgcaactacagcatcgattatcacaatcagatcttcgtgcagaacgccgaactgcacacccacggttttgttacgccggatctcggcatggcctgttaccgcaactcggtgctgctgcgcgaactgaccggacgcgaggtctacccgatcgagcgtcagattgccttccagaccttcccggctaaatcggagcactgagatgagttcacaaacactgttcagcgcagcccgccccgcaggccagttcaccctgaggcccatgctcgccagcgatgcgccgctgattcatcgctgggtcacccgcgattacgcccgcttctggggcatgcagcagcagagcctgaatcaggtcgctgagttctaccagacgctgaccaaagccgatccccatgctgcgctgattggttgctgcaacgaccagccggtctgcctgattgagtgctatcgcgcctgcgacgatgaggtcggcaaattttatcctgccgcacccgatgattacggcatgcatattctgattgcccccgccagccagccgatcagagccttcagctggcaggtcttcacgctggtgatggactacatgttcagccgccccgaggtgcgccgcgtggtggtagagccagatgtgcgcaatgagaagattcacgtgctgaataagcgtgccgggttccgctatcagcacaccatcgacatgggtcataaaaccgcctggctggccttttgccagcgggaagattatcaacacgcgttactgcaggagtcgcagactatgaacaccccctcttcgctgatgaacggtacacatctgacgggcgaccaatgggcgcaggcaaaccgcatgctgatccggaaagcgattgccgaattcgcgcatgagaaaatcattacgccacaggagtcgggcgacggcagctatcagcttgcggtgccaggcggtgaagcgagctaccatttccgggcgtcgcggctggcgctggatcactgggaaattgacgccggctcactgcgcaagcaggagaacggtcgtgatctgccgctggatgcgctgaagtttatcgtggagtttaacggggagatcggtattacacaggcactgctggcgacctacatggaagagatcagcagcacgctgtgcagtagcgtttataagctgcagaagaataatcctgacagccaggcgctggtctcagccgatttccagacggtggaagcggcgatgaccgaaggccatccctgctttgttgccaacaacgggcgtattggcttcgatgcccatgattatctggcgtatgcgcccgaggccgccgcgcccgttcacctcatctgggtcgcggttcatcgccgcaacgcccatttctccagcctcagcgagctgagctatgagcagttgatgcgtgatgaactgggcgacaccaccctggagcagtttactgcgcagctggtgaataaagggctggcggttgaggagtatatcctgatgccggtgcatccgtggcagtggcagaacaaactgctgaccgtatttgcggcggacatcgccagtcacgatattgtttatctcggcaccggagacgatgcttaccaggcgcagcagtcgatccgcaccttctttaaccgcagccagcccacgaagcgctacgtgaaaaccgccctgtcggtgctgaacatgggctttatgcgcggactgtccccctactatatggcgacaacgcctgcgataaacggctggctggaacaactggttgctggcgatgcgtggctgaatcgctgtgatttccgcatcctgcgcgaagtcgcggcggtcggctatcacaaccgctactatgagcaggcgatcaaaggcgactccgcctataaaaagatgttcgctgcgctgtggcgggataatcctgcggccagcctgcagcccggccagcgtctgatgaccatggcctctttcctgcacgtcgatcaccatcagcaggcactgctgccagcgctgatagcggattctggtttgcccgcgaagcagtggatagaacgctacctgacctgctatctcagcccgctgctgcactgcttctatcagcatgacctggtgtttatgccgcacggtgaaaacctcatcatgctgctggagaacaacgtgccggtcagtgcatacatgaaggatatcggcgaagagatcgccgtgatgaatccggatgcggtgctgccggaaggcgtgcagcgtctggcggtggatgtgccggagaacctgaagctgctctcgatcttcaccgatgtgtttgactgcatcttccgctttatcagcgcgattctgcatcagtcaggaacgctgccggaggaagaattctggcaggcggtggcgcagtgcgtgaaggattaccagcaggcgcacccggagcatgccactaagttcgcgcgctatgacatgttcgcgccggagtttacccgctcctgtctgaaccgcctgcagctggcgaataaccagcagatgataaacctcgccgatcccgctgagaatctcaagttcgccggaacgctgaataacccgattgcccgctggcgataagcgttacaccgtcacgctgaaattatgcggtcaccctggccgctgtgtgccgttgataaaatataacccagcaggatggcagaaatttatttgccatcctgcaactgaaacaaagagattacatcccacaagctt

SEQ ID NO: 42 - DNA, 10427 bp, synthetic construct
  misc_feature 1..10427: note = Vector Construct
  source 1..10427: mol_type = other DNA
aattagagcaggtacgtgcgtctgcgctgaacgcggcgaaacagtgagagctctgaaatgaataattccgtttacgattttattggtatcggtatcggccctttcaacctgagtctggcctgtctgagtgagccggttgagggactgaacggtcttttcctggatgagaacccaggctttgactggcacaccggcatgatgctggaaaatgcccatctgcagacgccgtttatggccgatctggtgacactcgccgatccgaccagcccttacagcctgcttaactacatgaaggaaaaggggaaattatactctttttatatccgtgaagacttcttcctgatgcgcaaagagtataatcagtactgccagtgggccagttcacggctctctaatctgcgctggaacagccacgttgagtatgtcagctacgatgaggcggctggcgtctatcaactccaggtgaccgacacccggaatggtcagaaacagaactatctggcacgacatctgatcctcggcaccgggcctaaagcctggatgccggagtgcagccagccgcaccgccagcgcctgacgcactcaagccactatctggtcaacaaagcggaactgcagcagaagcgttccatcaccgtgctgggcagcggccagagcgcggcagaaatctactacgacctgctgaccgatatcgaccgctttggctatcagctcaactggatcacccgcgcgccgcgcttctatccgctggagtacaccaagctcacgctggagatgacttcgccggagtgggtcgactatttccacgcgctgcccgccagcacccgcgacgaactgaatgcgcgccacaagaatctctacaaaggcatcaacagcagcctgattaacgacatttatgacctgatgtatgtcaaacagctcgacggcgatctgaacgttaacctgtttactcactcggcgctgaccgctatgcgctggctgccacagggagagttcgagctgacgctgcatcaggaggagcaggattgcaccttcacccgtcgcaccgaaggactggtgatggcgaccggctatcactaccagccaccgatgtttttagagggcatcaacgcccgcctgcgctgggatgacaaaggccgctacgacgtgcagcgcaactacagcatcgattatcacaatcagatcttcgtgcagaacgccgaactgcacacccacggttttgttacgccggatctcggcatggcctgttaccgcaactcggtgctgctgcgcgaactgaccggacgcgaggtctacccgatcgagcgtcagattgccttccagaccttcccggctaaatcggagcactgagatgagttcacaaacactgttcagcgcagcccgccccgcaggccagttcaccctgaggcccatgctcgccagcgatgcgccgctgattcatcgctgggtcacccgcgattacgcccgcttctggggcatgcagcagcagagcctgaatcaggtcgctgagttctaccagacgctgaccaaagccgatccccatgctgcgctgattggttgctgcaacgaccagccggtctgcctgattgagtgctatcgcgcctgcgacgatgaggtcggcaaattttatcctgccgcacccgatgattacggcatgcatattctgattgcccccgccagccagccgatcagagccttcagctggcaggtcttcacgctggtgatggactacatgttcagccgccccgaggtgcgccgcgtggtggtagagccagatgtgcgcaatgagaagattcacgtgctgaataagcgtgccgggttccgctatcagcacaccatcgacatgggtcataaaaccgcctggctggccttttgccagcgggaagattatcaacacgcgttactgcaggagtcgcagactatgaacaccccctcttcgctgatgaacggtacacatctgacgggcgaccaatgggcgcaggcaaaccgcatgctgatccggaaagcgattgccgaattcgcgcatgagaaaatcattacgccacaggagtcgggcgacggcagctatcagcttgcggtgccaggcggtgaagcgagctaccatttccgggcgtcgcggctggcgctggatcactgggaaattgacgccggctcactgcgcaagcaggagaacggtcgtgatctgccgctggatgcgctgaagtttatcgtggagtttaacggggagatcggtattacacaggcactgctggcgacctacatggaagagatcagcagcacgctgtgcagtagcgtttataagctgcagaagaataatcctgacagccaggcgctggtctcagccgatttccagacggtggaagcggcgatgaccgaaggccatccctgctttgttgccaacaacgggcgtattggcttcgatgcccatgattatctggcgtatgcgcccgaggccgccgcgcccgttcacctcatctgggtcgcggttcatcgccgcaacgcccatttctccagcctcagcgagctgagctatgagcagttgatgcgtgatgaactgggcgacaccaccctggagcagtttactgcgcagctggtgaataaagggctggcggttgaggagtatatcctgatgccggtgcatccgtggcagtggcagaacaaactgctgaccgtatttgcggcggacatcgccagtcacgatattgtttatctcggcaccggagacgatgcttaccaggcgcagcagtcgatccgcaccttctttaaccgcagccagcccacgaagcgctacgtgaaaaccgccctgtcggtgctgaacatgggctttatgcgcggactgtccccctactatatggcgacaacgcctgcgataaacggctggctggaacaactggttgctggcgatgcgtggctgaatcgctgtgatttccgcatcctgcgcgaagtcgcggcggtcggctatcacaaccgctactatgagcaggcgatcaaaggcgactccgcctataaaaagatgttcgctgcgctgtggcgggataatcctgcggccagcctgcagcccggccagcgtctgatgaccatggcctctttcctgcacgtcgatcaccatcagcaggcactgctgccagcgctgatagcggattctggtttgcccgcgaagcagtggatagaacgctacctgacctgctatctcagcccgctgctgcactgcttctatcagcatgacctggtgtttatgccgcacggtgaaaacctcatcatgctgctggagaacaacgtgccggtcagtgcatacatgaaggatatcggcgaagagatcgccgtgatgaatccggatgcggtgctgccggaaggcgtgcagcgtctggcggtggatgtgccggagaacctgaagctgctctcgatcttcaccgatgtgtttgactgcatcttccgctttatcagcgcgattctgcatcagtcaggaacgctgccggaggaagaattctggcaggcggtggcgcagtgcgtgaaggattaccagcaggcgcacccggagcatgccactaagttcgcgcgctatgacatgttcgcgccggagtttacccgctcctgtctgaaccgcctgcagctggcgaataaccagcagatgataaacctcgccgatcccgctgagaatctcaagttcgccggaacgctgaataacccgattgcccgctggcgataagcgttacaccgtcacgctgaaattatgcggtcaccctggccgctgtgtgccgttgataaaatataacccagcaggatggcagaaatttatttgccatcctgcaactgaaacaaagagattacatcccactgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 43 - DNA, 1344 bp, Corynebacterium xerosis
  source 1..1344: mol_type = genomic DNA
gtgcacgacgaggagatccacgacctcatcggcatcgggatcggccccttcgggctgggactggctgcgcttgccgacccgctgccggacgtcgacgccgtgttcctggaccaggccgacaggttctcctggcacccgggactgatgatcgagggcaccaccctccaggtccccttcctcgcggacctcgtgacgatggcggatcccacctcgccctactccttcctcgccttcctcaagcagaccggcaggctctaccccttctacatccgggaatccttctacccgctgcgcagcgagtacgacgagtactgccgctgggtcgccgcacagctggactgcctgcgctggcggcgtcgggtggtcgccgtggaggaggagggcgatcacctgctcgtcacggccgccgtgctcgatgcaaccggggctgtgatcggctgcgagcacctgcgcggccgccacgtggctctcgccgtcggcaccgcgccgcaccttccgccgcccctcgccgagctgatcggcccggccggtggtgctccagcagacgaggccccagcccccgtgctgcacagcgccgactacctccggcggaaggacgagctgctcgccgccggatccgtgacggtggtgggcagcgggcagtccgccgcagagatctatcgggacctgctccaggagggcggggccgacgggctgcgggtggactgggtgacccgttccccgcggttcttccccatggagtacacgaagctcaccctcgagatgacctcgccggagtacaccgacctgcaccgctcccttcccgaggggacgcgcgaccgcctggcgcgggagcagcgtgccctgcacaagggcatcagcgcggacctgatcgacgagatccacgagctcctctaccgcctctccgccgggggccgggaggtgccctcgagcctgctcacgggagccgccgtccgcgccgcccgcagggactccgggagcggacggatcgtcctggacctcgagcatccgctcacgggctcggtcgcggagcatcgcaccggcgccgtggtcgccgcgaccggctaccggccccgctccacggagctgctcgcaccgctcggggaccggatccgccacgacgacgcgggccggctcgacgtcacccgcgactacaccgtcgaccgcgaggagcggatccatgtgctgggcggcgaggagcacacccacggcgtcaccgcccctgacctcggcttcggcaccttgcgcgcctccgtgatcctcgccgcgctcaccgggcgggagccgtatccgatcgagcgtcgcatcgccttccagaccttcggcctccccgccgcccgctga

SEQ ID NO: 44 - DNA, 2430 bp, Corynebacterium xerosis
  source 1..2430: mol_type = genomic DNA
atgaacctgatgaacccgaacgacccgttggacctgagcgtcgaaccgctccgtcctgcggaccacaccggcctgctgcacagctggctgagcaccccgcgcgcccgtttctggggcatggtcgaccattcgcccgcacaggtgcgcgcctacctcgacgccgtcgccgcctccgagcacgagaacggccggatcggcctgcgggacggggtcccgctcgtctacgtcgagacctatgatcccgcccgactgctgcccgccggggtgctgcatccggaacccggcgatctcggcatgcacctcctcatcgcgccgccggacggcccgaccgagcacggcctcaccgccgcggtgctggcaggcgtcgtgcgctggtgcttcgagaaccgtggcgccgcccggatcgtcgtcgaacccgacgaacgcaacagcgcagtgctcgccaagaacgccgcagccggtttccgcgtgctgcgcaccgtcgaactgcccgagggatcgggcacgaagcgcgcggtgctgagcatctgcacccgggcggacttcgcggcttccccgctcggggaccggcggcagcgcaaggaactccggggcggcgccggagccgacgacgcgactgcggagcgcgcacatctcggcgctcccgtcgccccgacgtcccatctgcgcgccgacgtagccgagcgggcccaccggcacctgatcgccaaggccctcgccgagctcagccacgaacgagtcctcgcccccgaggcgctcggcgacgatcgcttcgccctggcagtccccggcaccgacgtacgctacgagttccgggcccgcgtgctcccgctcgagcactggctggtggacccggcctcgatccggcgcctcgaggacggtgccccgacggcgctggacgtgcagatcctcgtgttggagctgcaggatccgctcggcatcgccgacgatctgctctccacctacctcgaagagctcgcctccaccttcgccgcccgctgcgcgacgctggacgaggcccgtcgcggcgagcggccgtcggcagagggcctgctcacggcggacctcccggacgtcgaggccgccatgacggagggccacccgggcttcctcgcgaacaacgggcgcatcgggttctccctgcccgactaccgcgcctacgcaccggagcagggtcggcacacgcgcctgcagtgggtcgcggtgcacgccgcgcgcagcaccctgtccctcggcgaggggctggacgaggccgcgcatgcccgctggagcctctcgtccgaggagcgcgactccttcgcggaccgtctggccggccggggcctggacccgtcggactaccacctgctgcccctgcatccctggcaggcagagcatcgtctgcccatcaccttcgccccggacatcgcccgtggggacctcgtgcccctcggggcgggaggcgacgagatgcagccccagcagtccctgcgcaccttcctcaaccgcacgcgagcctgggcgccctatgtgaaggtcgccctcgcgatccagaacatggggttcctgcgcggcctctcgccgcagtacatgcgggacacccccacgatcaacgactggctcgccgcgctcgtggacagcgaccccgagttcgcgggcggcggcttccgcgtcctgcgggagcgggcagcactgggctacaccggggacgcctaccaccgcacgcccgccacgaatccccaccgcaagatgctcgcggcgctgtggcgggagagccccctgccgctgctcgagccgggggagcgggccttcacgatggcggcgctcctgcaccgcgatcacgacggcacggcgctcgtgagcacgatgatccgcacctcggagctgccggcggaggtctgggtcgcccagtacctgcgcgcctacctccgcccgctcgtccatgcgctgctcgcgcacgacgtggtggtcatgccgcacggcgagaacctcatcctgcgggttcaggagcagcaggtggtcggggcattcctcaaggacatcggggaggagatggcggtgctgggccaccgcccgctgcctgcggtgatggagcgggtacgggccgtggtccccggcgaggacaaggccctgtcgatcttcaccgacgtcttcgacggggtgctgcggcatctcgcggggatcctcgacgccgacggggtgcttcccgccgatcgcttctggcgggtcgtggcgggcatcctggacgagtacgaggcggaccatccggatctcgcgcgggggatctccggggacatcgacctgcgtgcgggcaccttcgcgcactcgtgcctgaaccgcctgcagctgcggaacaccctgcagatggtcgacctcggcgaccaggcctcgtcgctgctcttcgccggggacatggacaacccggtggctcgggcgtccgtccctgcctga

SEQ ID NO: 45 - protein, 447 aa, Corynebacterium xerosis
VHDEEIHDLIGIGIGPFGLGLAALADPLPDVDAVFLDQADRFSWHPGLMIEGTTLQVPFLADLVTMADPTSPYSFLAFLKQTGRLYPFYIRESFYPLRSEYDEYCRWVAAQLDCLRWRRRVVAVEEEGDHLLVTAAVLDATGAVIGCEHLRGRHVALAVGTAPHLPPPLAELIGPAGGAPADEAPAPVLHSADYLRRKDELLAAGSVTVVGSGQSAAEIYRDLLQEGGADGLRVDWVTRSPRFFPMEYTKLTLEMTSPEYTDLHRSLPEGTRDRLAREQRALHKGISADLIDEIHELLYRLSAGGREVPSSLLTGAAVRAARRDSGSGRIVLDLEHPLTGSVAEHRTGAVVAATGYRPRSTELLAPLGDRIRHDDAGRLDVTRDYTVDREERIHVLGGEEHTHGVTAPDLGFGTLRASVILAALTGREPYPIERRIAFQTFGLPAAR

SEQ ID NO: 46 - protein, 809 aa, Corynebacterium xerosis
MNLMNPNDPLDLSVEPLRPADHTGLLHSWLSTPRARFWGMVDHSPAQVRAYLDAVAASEHENGRIGLRDGVPLVYVETYDPARLLPAGVLHPEPGDLGMHLLIAPPDGPTEHGLTAAVLAGVVRWCFENRGAARIVVEPDERNSAVLAKNAAAGFRVLRTVELPEGSGTKRAVLSICTRADFAASPLGDRRQRKELRGGAGADDATAERAHLGAPVAPTSHLRADVAERAHRHLIAKALAELSHERVLAPEALGDDRFALAVPGTDVRYEFRARVLPLEHWLVDPASIRRLEDGAPTALDVQILVLELQDPLGIADDLLSTYLEELASTFAARCATLDEARRGERPSAEGLLTADLPDVEAAMTEGHPGFLANNGRIGFSLPDYRAYAPEQGRHTRLQWVAVHAARSTLSLGEGLDEAAHARWSLSSEERDSFADRLAGRGLDPSDYHLLPLHPWQAEHRLPITFAPDIARGDLVPLGAGGDEMQPQQSLRTFLNRTRAWAPYVKVALAIQNMGFLRGLSPQYMRDTPTINDWLAALVDSDPEFAGGGFRVLRERAALGYTGDAYHRTPATNPHRKMLAALWRESPLPLLEPGERAFTMAALLHRDHDGTALVSTMIRTSELPAEVWVAQYLRAYLRPLVHALLAHDVVVMPHGENLILRVQEQQVVGAFLKDIGEEMAVLGHRPLPAVMERVRAVVPGEDKALSIFTDVFDGVLRHLAGILDADGVLPADRFWRVVAGILDEYEADHPDLARGISGDIDLRAGTFAHSCLNRLQLRNTLQMVDLGDQASSLLFAGDMDNPVARASVPA

SEQ ID NO: 47 - DNA, 3922 bp, synthetic construct
  misc_feature 1..3922: note = Vector Construct
  source 1..3922: mol_type = other DNA
aagcttgcgaccggggtggaccgatgagcgcccgggccacgccggaggcggagagcccggaacccgcggtgcacgacgaggagatccacgacctcatcggcatcgggatcggccccttcgggctgggactggctgcgcttgccgacccgctgccggacgtcgacgccgtgttcctggaccaggccgacaggttctcctggcacccgggactgatgatcgagggcaccaccctccaggtccccttcctcgcggacctcgtgacgatggcggatcccacctcgccctactccttcctcgccttcctcaagcagaccggcaggctctaccccttctacatccgggaatccttctacccgctgcgcagcgagtacgacgagtactgccgctgggtcgccgcacagctggactgcctgcgctggcggcgtcgggtggtcgccgtggaggaggagggcgatcacctgctcgtcacggccgccgtgctcgatgcaaccggggctgtgatcggctgcgagcacctgcgcggccgccacgtggctctcgccgtcggcaccgcgccgcaccttccgccgcccctcgccgagctgatcggcccggccggtggtgctccagcagacgaggccccagcccccgtgctgcacagcgccgactacctccggcggaaggacgagctgctcgccgccggatccgtgacggtggtgggcagcgggcagtccgccgcagagatctatcgggacctgctccaggagggcggggccgacgggctgcgggtggactgggtgacccgttccccgcggttcttccccatggagtacacgaagctcaccctcgagatgacctcgccggagtacaccgacctgcaccgctcccttcccgaggggacgcgcgaccgcctggcgcgggagcagcgtgccctgcacaagggcatcagcgcggacctgatcgacgagatccacgagctcctctaccgcctctccgccgggggccgggaggtgccctcgagcctgctcacgggagccgccgtccgcgccgcccgcagggactccgggagcggacggatcgtcctggacctcgagcatccgctcacgggctcggtcgcggagcatcgcaccggcgccgtggtcgccgcgaccggctaccggccccgctccacggagctgctcgcaccgctcggggaccggatccgccacgacgacgcgggccggctcgacgtcacccgcgactacaccgtcgaccgcgaggagcggatccatgtgctgggcggcgaggagcacacccacggcgtcaccgcccctgacctcggcttcggcaccttgcgcgcctccgtgatcctcgccgcgctcaccgggcgggagccgtatccgatcgagcgtcgcatcgccttccagaccttcggcctccccgccgcccgctgaccaggagcgtccccatgaacctgatgaacccgaacgacccgttggacctgagcgtcgaaccgctccgtcctgcggaccacaccggcctgctgcacagctggctgagcaccccgcgcgcccgtttctggggcatggtcgaccattcgcccgcacaggtgcgcgcctacctcgacgccgtcgccgcctccgagcacgagaacggccggatcggcctgcgggacggggtcccgctcgtctacgtcgagacctatgatcccgcccgactgctgcccgccggggtgctgcatccggaacccggcgatctcggcatgcacctcctcatcgcgccgccggacggcccgaccgagcacggcctcaccgccgcggtgctggcaggcgtcgtgcgctggtgcttcgagaaccgtggcgccgcccggatcgtcgtcgaacccgacgaacgcaacagcgcagtgctcgccaagaacgccgcagccggtttccgcgtgctgcgcaccgtcgaactgcccgagggatcgggcacgaagcgcgcggtgctgagcatctgcacccgggcggacttcgcggcttccccgctcggggaccggcggcagcgcaaggaactccggggcggcgccggagccgacgacgcgactgcggagcgcgcacatctcggcgctcccgtcgccccgacgtcccatctgcgcgccgacgtagccgagcgggcccaccggcacctgatcgccaaggccctcgccgagctcagccacgaacgagtcctcgcccccgaggcgctcggcgacgatcgcttcgccctggcagtccccggcaccgacgtacgctacgagttccgggcccgcgtgctcccgctcgagcactggctggtggacccggcctcgatccggcgcctcgaggacggtgccccgacggcgctggacgtgcagatcctcgtgttggagctgcaggatccgctcggcatcgccgacgatctgctctccacctacctcgaagagctcgcctccaccttcgccgcccgctgcgcgacgctggacgaggcccgtcgcggcgagcggccgtcggcagagggcctgctcacggcggacctcccggacgtcgaggccgccatgacggagggccacccgggcttcctcgcgaacaacgggcgcatcgggttctccctgcccgactaccgcgcctacgcaccggagcagggtcggcacacgcgcctgcagtgggtcgcggtgcacgccgcgcgcagcaccctgtccctcggcgaggggctggacgaggccgcgcatgcccgctggagcctctcgtccgaggagcgcgactccttcgcggaccgtctggccggccggggcctggacccgtcggactaccacctgctgcccctgcatccctggcaggcagagcatcgtctgcccatcaccttcgccccggacatcgcccgtggggacctcgtgcccctcggggcgggaggcgacgagatgcagccccagcagtccctgcgcaccttcctcaaccgcacgcgagcctgggcgccctatgtgaaggtcgccctcgcgatccagaacatggggttcctgcgcggcctctcgccgcagtacatgcgggacacccccacgatcaacgactggctcgccgcgctcgtggacagcgaccccgagttcgcgggcggcggcttccgcgtcctgcgggagcgggcagcactgggctacaccggggacgcctaccaccgcacgcccgccacgaatccccaccgcaagatgctcgcggcgctgtggcgggagagccccctgccgctgctcgagccgggggagcgggccttcacgatggcggcgctcctgcaccgcgatcacgacggcacggcgctcgtgagcacgatgatccgcacctcggagctgccggcggaggtctgggtcgcccagtacctgcgcgcctacctccgcccgctcgtccatgcgctgctcgcgcacgacgtggtggtcatgccgcacggcgagaacctcatcctgcgggttcaggagcagcaggtggtcggggcattcctcaaggacatcggggaggagatggcggtgctgggccaccgcccgctgcctgcggtgatggagcgggtacgggccgtggtccccggcgaggacaaggccctgtcgatcttcaccgacgtcttcgacggggtgctgcggcatctcgcggggatcctcgacgccgacggggtgcttcccgccgatcgcttctggcgggtcgtggcgggcatcctggacgagtacgaggcggaccatccggatctcgcgcgggggatctccggggacatcgacctgcgtgcgggcaccttcgcgcactcgtgcctgaaccgcctgcagctgcggaacaccctgcagatggtcgacctcggcgaccaggcctcgtcgctgctcttcgccggggacatggacaacccggtggctcgggcgtccgtccctgcctgaggtccggggcggcggcgctcagctctccaggagccgcgccgccgccgcgagggtgtcgccaagctt

SEQ ID NO: 48 - DNA, 10507 bp, synthetic construct
  misc_feature 1..10507: note = Vector Sequence
  source 1..10507: mol_type = other DNA
aattagcgaccggggtggaccgatgagcgcccgggccacgccggaggcggagagcccggaacccgcggtgcacgacgaggagatccacgacctcatcggcatcgggatcggccccttcgggctgggactggctgcgcttgccgacccgctgccggacgtcgacgccgtgttcctggaccaggccgacaggttctcctggcacccgggactgatgatcgagggcaccaccctccaggtccccttcctcgcggacctcgtgacgatggcggatcccacctcgccctactccttcctcgccttcctcaagcagaccggcaggctctaccccttctacatccgggaatccttctacccgctgcgcagcgagtacgacgagtactgccgctgggtcgccgcacagctggactgcctgcgctggcggcgtcgggtggtcgccgtggaggaggagggcgatcacctgctcgtcacggccgccgtgctcgatgcaaccggggctgtgatcggctgcgagcacctgcgcggccgccacgtggctctcgccgtcggcaccgcgccgcaccttccgccgcccctcgccgagctgatcggcccggccggtggtgctccagcagacgaggccccagcccccgtgctgcacagcgccgactacctccggcggaaggacgagctgctcgccgccggatccgtgacggtggtgggcagcgggcagtccgccgcagagatctatcgggacctgctccaggagggcggggccgacgggctgcgggtggactgggtgacccgttccccgcggttcttccccatggagtacacgaagctcaccctcgagatgacctcgccggagtacaccgacctgcaccgctcccttcccgaggggacgcgcgaccgcctggcgcgggagcagcgtgccctgcacaagggcatcagcgcggacctgatcgacgagatccacgagctcctctaccgcctctccgccgggggccgggaggtgccctcgagcctgctcacgggagccgccgtccgcgccgcccgcagggactccgggagcggacggatcgtcctggacctcgagcatccgctcacgggctcggtcgcggagcatcgcaccggcgccgtggtcgccgcgaccggctaccggccccgctccacggagctgctcgcaccgctcggggaccggatccgccacgacgacgcgggccggctcgacgtcacccgcgactacaccgtcgaccgcgaggagcggatccatgtgctgggcggcgaggagcacacccacggcgtcaccgcccctgacctcggcttcggcaccttgcgcgcctccgtgatcctcgccgcgctcaccgggcgggagccgtatccgatcgagcgtcgcatcgccttccagaccttcggcctccccgccgcccgctgaccaggagcgtccccatgaacctgatgaacccgaacgacccgttggacctgagcgtcgaaccgctccgtcctgcggaccacaccggcctgctgcacagctggctgagcaccccgcgcgcccgtttctggggcatggtcgaccattcgcccgcacaggtgcgcgcctacctcgacgccgtcgccgcctccgagcacgagaacggccggatcggcctgcgggacggggtcccgctcgtctacgtcgagacctatgatcccgcccgactgctgcccgccggggtgctgcatccggaacccggcgatctcggcatgcacctcctcatcgcgccgccggacggcccgaccgagcacggcctcaccgccgcggtgctggcaggcgtcgtgcgctggtgcttcgagaaccgtggcgccgcccggatcgtcgtcgaacccgacgaacgcaacagcgcagtgctcgccaagaacgccgcagccggtttccgcgtgctgcgcaccgtcgaactgcccgagggatcgggcacgaagcgcgcggtgctgagcatctgcacccgggcggacttcgcggcttccccgctcggggaccggcggcagcgcaaggaactccggggcggcgccggagccgacgacgcgactgcggagcgcgcacatctcggcgctcccgtcgccccgacgtcccatctgcgcgccgacgtagccgagcgggcccaccggcacctgatcgccaaggccctcgccgagctcagccacgaacgagtcctcgcccccgaggcgctcggcgacgatcgcttcgccctggcagtccccggcaccgacgtacgctacgagttccgggcccgcgtgctcccgctcgagcactggctggtggacccggcctcgatccggcgcctcgaggacggtgccccgacggcgctggacgtgcagatcctcgtgttggagctgcaggatccgctcggcatcgccgacgatctgctctccacctacctcgaagagctcgcctccaccttcgccgcccgctgcgcgacgctggacgaggcccgtcgcggcgagcggccgtcggcagagggcctgctcacggcggacctcccggacgtcgaggccgccatgacggagggccacccgggcttcctcgcgaacaacgggcgcatcgggttctccctgcccgactaccgcgcctacgcaccggagcagggtcggcacacgcgcctgcagtgggtcgcggtgcacgccgcgcgcagcaccctgtccctcggcgaggggctggacgaggccgcgcatgcccgctggagcctctcgtccgaggagcgcgactccttcgcggaccgtctggccggccggggcctggacccgtcggactaccacctgctgcccctgcatccctggcaggcagagcatcgtctgcccatcaccttcgccccggacatcgcccgtggggacctcgtgcccctcggggcgggaggcgacgagatgcagccccagcagtccctgcgcaccttcctcaaccgcacgcgagcctgggcgccctatgtgaaggtcgccctcgcgatccagaacatggggttcctgcgcggcctctcgccgcagtacatgcgggacacccccacgatcaacgactggctcgccgcgctcgtggacagcgaccccgagttcgcgggcggcggcttccgcgtcctgcgggagcgggcagcactgggctacaccggggacgcctaccaccgcacgcccgccacgaatccccaccgcaagatgctcgcggcgctgtggcgggagagccccctgccgctgctcgagccgggggagcgggccttcacgatggcggcgctcctgcaccgcgatcacgacggcacggcgctcgtgagcacgatgatccgcacctcggagctgccggcggaggtctgggtcgcccagtacctgcgcgcctacctccgcccgctcgtccatgcgctgctcgcgcacgacgtggtggtcatgccgcacggcgagaacctcatcctgcgggttcaggagcagcaggtggtcggggcattcctcaaggacatcggggaggagatggcggtgctgggccaccgcccgctgcctgcggtgatggagcgggtacgggccgtggtccccggcgaggacaaggccctgtcgatcttcaccgacgtcttcgacggggtgctgcggcatctcgcggggatcctcgacgccgacggggtgcttcccgccgatcgcttctggcgggtcgtggcgggcatcctggacgagtacgaggcggaccatccggatctcgcgcgggggatctccggggacatcgacctgcgtgcgggcaccttcgcgcactcgtgcctgaaccgcctgcagctgcggaacaccctgcagatggtcgacctcggcgaccaggcctcgtcgctgctcttcgccggggacatggacaacccggtggctcgggcgtccgtccctgcctgaggtccggggcggcggcgctcagctctccaggagccgcgccgccgccgcgagggtgtcgcctgcatgcctgcaggtcgactctagaggatccccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

SEQ ID NO: 49 - DNA, 2142 bp, Escherichia coli
  source 1..2142: mol_type = genomic DNA
atgaacatcattgccattatgggaccgcatggcgtcttttataaagatgagcccatcaaagaactggagtcggcgctggtggcgcaaggctttcagattatctggccacaaaacagcgttgatttgctgaaatttatcgagcataaccctcgaatttgcggcgtgatttttgactgggatgagtacagtctcgatttatgtagcgatatcaatcagcttaatgaatatctcccgctttatgccttcatcaacacccactcgacgatggatgtcagcgtgcaggatatgcggatggcgctctggttttttgaatatgcgctggggcaggcggaagatatcgccattcgtatgcgtcagtacaccgacgaatatcttgataacattacaccgccgttcacgaaagccttgtttacctacgtcaaagagcggaagtacaccttttgtacgccggggcatatgggcggcaccgcatatcaaaaaagcccggttggctgtctgttttatgattttttcggcgggaatactcttaaggctgatgtctctatttcggtcaccgagcttggttcgttgctcgaccacaccgggccacacctggaagcggaagagtacatcgcgcggacttttggcgcggaacagagttatatcgttaccaacggaacatcgacgtcgaacaaaattgtgggtatgtacgccgcgccatccggcagtacgctgttgatcgaccgcaattgtcataaatcgctggcgcatctgttgatgatgaacgatgtagtgccagtctggctgaaaccgacgcgtaatgcgttggggattcttggtgggatcccgcgccgtgaatttactcgcgacagcatcgaagagaaagtcgctgctaccacgcaagcacaatggccggttcatgcggtgatcaccaactccacctatgatggcttgctctacaacaccgactggatcaaacagacgctggatgtcccgtcgattcacttcgattctgcctgggtgccgtacacccattttcatccgatctaccagggtaaaagtggtatgagcggcgagcgtgttgcgggaaaagtgatcttcgaaacgcaatcgacccacaaaatgctggcggcgttatcgcaggcttcgctgatccacattaaaggcgagtatgacgaagaggcctttaacgaagcctttatgatgcataccaccacctcgcccagttatcccattgttgcttcggttgagacggcggcggcgatgctgcgtggtaatccgggcaaacggctgattaaccgttcagtagaacgagctctgcattttcgcaaagaggtccagcggctgcgggaagagtctgacggttggtttttcgatatctggcaaccgccgcaggtggatgaagccgaatgctggcccgttgcgcctggcgaacagtggcacggctttaacgatgcggatgccgatcatatgtttctcgatccggttaaagtcactattttgacaccggggatggacgagcagggcaatatgagcgaggaggggatcccggcggcgctggtagcaaaattcctcgacgaacgtgggatcgtagtagagaaaaccggcccttataacctgctgtttctctttagtattggcatcgataaaaccaaagcaatgggattattgcgtgggttgacggaattcaaacgctcttacgatctcaacctgcggatcaaaaatatgctacccgatctctatgcagaagatcccgatttctaccgcaatatgcgtattcaggatctggcacaagggatccataagctgattcgtaaacacgatcttcccggtttgatgttgcgggcattcgatactttgccggagatgatcatgacgccacatcaggcatggcaacgacaaattaaaggcgaagtagaaaccattgcgctggaacaactggtcggtagagtatcggcaaatatgatcctgccttatccaccgggcgtaccgctgttgatgcctggagaaatgctgaccaaagagagccgcacagtactcgattttctactgatgctttgttccgtcgggcaacattaccccggttttgaaacggatattcacggcgcgaaacaggacgaagacggcgtttaccgcgtacgagtcctaaaaatggcgggataa

SEQ ID NO: 50 - protein, 713 aa, Escherichia coli
MNIIAIMGPHGVFYKDEPIKELESALVAQGFQIIWPQNSVDLLKFIEHNPRICGVIFDWDEYSLDLCSDINQLNEYLPLYAFINTHSTMDVSVQDMRMALWFFEYALGQAEDIAIRMRQYTDEYLDNITPPFTKALFTYVKERKYTFCTPGHMGGTAYQKSPVGCLFYDFFGGNTLKADVSISVTELGSLLDHTGPHLEAEEYIARTFGAEQSYIVTNGTSTSNKIVGMYAAPSGSTLLIDRNCHKSLAHLLMMNDVVPVWLKPTRNALGILGGIPRREFTRDSIEEKVAATTQAQWPVHAVITNSTYDGLLYNTDWIKQTLDVPSIHFDSAWVPYTHFHPIYQGKSGMSGERVAGKVIFETQSTHKMLAALSQASLIHIKGEYDEEAFNEAFMMHTTTSPSYPIVASVETAAAMLRGNPGKRLINRSVERALHFRKEVQRLREESDGWFFDIWQPPQVDEAECWPVAPGEQWHGFNDADADHMFLDPVKVTILTPGMDEQGNMSEEGIPAALVAKFLDERGIVVEKTGPYNLLFLFSIGIDKTKAMGLLRGLTEFKRSYDLNLRIKNMLPDLYAEDPDFYRNMRIQDLAQGIHKLIRKHDLPGLMLRAFDTLPEMIMTPHQAWQRQIKGEVETIALEQLVGRVSANMILPYPPGVPLLMPGEMLTKESRTVLDFLLMLCSVGQHYPGFETDIHGAKQDEDGVYRVRVLKMAG

SEQ ID NO: 51 - DNA, 2433 bp, synthetic construct
  misc_feature 1..2433: note = Vector Construct
  source 1..2433: mol_type = other DNA
gcaagcaggatgtcaggaggaccggtgttttagcgtgtcagtaggcgcgtagggtaagtggggtagcggcttgttagatatcttgaaatcggctttcaacagcattgatttcgatgtatttagctggccgttaccctgcgaatgtccacagggtagctggtagtttgaaaatcaacgccgttgcccttaggattcagtaactggcaaggaaggattttccaggaggaacacatgaacatcatcgctatcatgggccctcacggtgttttctacaaggatgagccaatcaaggagctggaatccgcactagttgcacagggctttcagatcatctggccccagaactccgttgaccttctcaaattcatcgagcacaatcctcgcatttgtggtgtgatttttgactgggacgagtactctcttgatttatgctccgacatcaaccagctcaacgagtacctgccactctacgcattcatcaacactcactccaccatggacgtttccgtgcaggacatgcgtatggcactctggttctttgaatacgctctgggccaggctgaggacatcgcgatccgcatgcgtcagtacaccgacgagtacctggacaacatcacccctccattcaccaaggctctcttcacctacgtaaaggaacgcaagtacactttctgcaccccaggccacatgggcggcaccgcctaccagaagtccccagtcggatgcctcttctacgacttcttcggcggtaacactcttaaggcagatgtctccatttccgtcaccgagttgggctctctgctggaccacaccggccctcacctggaggcagaagagtacatcgctcgtaccttcggtgctgaacagtcctacatcgtcaccaacggtacttccaccagcaacaagatcgttggtatgtacgcagctccttctggctccaccctgttgatcgaccgcaactgtcacaagtccctcgcgcatcttcttatgatgaacgatgtggtccctgtatggctgaagccaacccgtaacgctctgggcatccttggcggtatcccccgtcgcgagttcacccgtgattccatcgaggaaaaggttgcagccactacccaggcacagtggcctgtccacgctgtcattaccaactcgacctacgacggcctgctctacaacaccgattggatcaagcagaccctagatgttccttccattcacttcgacagcgcatgggttccttacactcacttccacccaatctaccagggtaagtccggaatgtccggcgagcgtgtcgctggcaaggttatcttcgaaacccaatcaacccacaagatgctggctgctctctcccaggcttctctgatccacatcaagggcgagtacgacgaggaagctttcaacgaggctttcatgatgcacaccaccacctccccatcctaccctatcgtcgcgtccgtcgagactgctgccgcaatgcttcgcggtaacccaggtaagcgcctcatcaaccgttccgttgagcgcgctcttcacttccgtaaggaagtgcagcgcctgcgtgaggaatctgacggttggttcttcgacatttggcagccacctcaggttgatgaggccgagtgctggccagttgctccaggtgaacagtggcacggattcaacgatgcagatgctgaccacatgtttttggacccggtcaaggtcaccattcttactcctggtatggatgagcagggcaacatgtctgaggagggtatcccagctgctctggttgcaaagttcctcgacgaacgtggcatcgttgttgagaagaccggaccatacaacctgctgttcctgttcagcatcggcatcgacaaaaccaaggcaatgggtctgctgcgcggccttaccgagttcaagcgctcctacgacctgaaccttcgcatcaagaatatgctgccggacctgtacgctgaagatcctgatttctaccgcaacatgcgcatccaggacctcgcacagggcatccacaagctcattcgcaagcacgacctgccaggccttatgctccgtgcattcgataccctcccagagatgatcatgacccctcaccaggcttggcagcgccagatcaagggcgaggtggaaaccatcgcactggagcagctggttggtcgtgtctccgccaacatgatcctgccatatccacctggcgttccgctgctgatgccaggcgagatgctcaccaaggagtcccgcaccgtattggacttccttctcatgttgtgctctgttggccagcactacccaggcttcgagaccgacatccacggcgctaagcaagatgaagacggcgtttaccgcgttcgcgtccttaagatggcaggctaacttgccagagcggcttccgggcgagtaacgtgcggatccgcctccacagttttcccggtt

SEQ ID NO: 52 - DNA, 1046 bp, synthetic construct
  misc_feature 1..1046: note = Vector Construct
  source 1..1046: mol_type = other DNA
acagctatgacatgattacggacggcgaattcctcctcgttgccacctccggcggctacggcaagcgcaccccacttgaggattactccacccagggccgtggtggcctcggcgtggtgaccttcaagtacaccccgaagcgcggtcgcctcgtcagcgccatcgcagttgaggaagatgacgagatcttcgccatcacctccgccggcggcgttgttcgcaccgaagtcaagcagatccgaccatcctcccgtgcaacaatgggtgttcgactggtcaacttggaagaaggtgtagaactgcttgccatcgacaagaacgtcgaagaccagggcgaagcatccgcagaagcagtagcaaagggtgcagtcgaaggaccagcatccaagactgctgccgaagaaaccgactccgttgacaacggatccgacgaaaacggcgaggaataatttatggcatcccgagaagtatccatcacccgaatctcaccactagcaaccttccgagttgcactggcaatgtccatcatcggactcgtggcgtggatcatctgcgtaaccgtcctctatttcggacttaacgtggctggcgtctggcagaacttcaacgatgtcatcggcggtgttggtgcagaacaaaccatcaccttcgggctcgtcctgagcatttccgcacttctgggagctatcggagcgattaccgtcgctgtgcttgcaccattgtgtgcaatcatctacaactcgattgttgacctcttcggtggactgcagattcaactgcaagaagaagtagactaacctctgaaacacaaacccctagacactgctaaatatagcctttgaacaggggatttgttgtattgttaacttgaaggtagagtcttatctcgttcctagaggggcctatagctcagtcggttagagcgcatcgctgataacgatgaggtcgcaagttcgattcttgctaggcccaccaggaacaaaaaaggggcattagctcagttggtagagcacctgctttgcaagcaggatgtcaggaggcgcgcc

SEQ ID NO: 53 - DNA, 1028 bp, synthetic construct
  misc_feature 1..1028: note = Vector Construct
  source 1..1028: mol_type = other DNA
gcaggatgtcaggaggcgcgcctccacagttttcccggttcatcttgtttaaggtgggccgggatttttgttttttagtgtttctgggatacctcggtggcactccggcggcactctgggtcattggaaagctgtgtgtactttcgacaacaagatttgggcaaaatggtggtcaaaatcaggaaatcttgttgtcgaaagtacaactgggtcttcggttctatccgaggtgggtgagtagggtgcctgcaagtgcgatactggtgacaagtgtcgtggatccgaaagccagaatgaaaggcttgaccccaacatggatcaggttctggatttttacgccacacccgagaccaaacattgctgcagagagcaaggctgtctgtaggaaacctccagccgcaattacctcgtctgggagcgcaacagtggagcgcaaaactaccatcgccaagaatccaaggataaataggggaacgaccggtggtctctttccatcgggggacgtgtaaccctggcggcgctggcgaaaacttaaaatggcagcaatgggtgcaagtaggagtactcgggcgagtttcaccacaactgcaacacctaatgctccaccaccaatgactcctccagctgctactacttgggcgatttcatggatggatccgcctgcccacatcccaccgatttcaggggataaccccaagactttggttgcgaatgggataaaaggaatcatcagcgttccgaaaataacaacaagtgcaaccgcagtaacgacctcttcttcttcggaatcagttactccttcaacacctgccacggccgcagcgccacaaatagaaaagccacaagcaatcaacagaacttgggttggtttcattctgagcaggtgtcccatgaggatggtcccaaaaataccaccggcaacgatgcacacaatcaccgccagcatggggaaaccaagtgacaaaatatctgagaaaactaactgcagaccaaggaagacgatgaattcgagctcggtacccggggat

SEQ ID NO: 54 - DNA, 7727 bp, synthetic construct
  misc_feature 1..7727: note = Vector Construct
  source 1..7727: mol_type = other DNA
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacggacggcgaattcctcctcgttgccacctccggcggctacggcaagcgcaccccacttgaggattactccacccagggccgtggtggcctcggcgtggtgaccttcaagtacaccccgaagcgcggtcgcctcgtcagcgccatcgcagttgaggaagatgacgagatcttcgccatcacctccgccggcggcgttgttcgcaccgaagtcaagcagatccgaccatcctcccgtgcaacaatgggtgttcgactggtcaacttggaagaaggtgtagaactgcttgccatcgacaagaacgtcgaagaccagggcgaagcatccgcagaagcagtagcaaagggtgcagtcgaaggaccagcatccaagactgctgccgaagaaaccgactccgttgacaacggatccgacgaaaacggcgaggaataatttatggcatcccgagaagtatccatcacccgaatctcaccactagcaaccttccgagttgcactggcaatgtccatcatcggactcgtggcgtggatcatctgcgtaaccgtcctctatttcggacttaacgtggctggcgtctggcagaacttcaacgatgtcatcggcggtgttggtgcagaacaaaccatcaccttcgggctcgtcctgagcatttccgcacttctgggagctatcggagcgattaccgtcgctgtgcttgcaccattgtgtgcaatcatctacaactcgattgttgacctcttcggtggactgcagattcaactgcaagaagaagtagactaacctctgaaacacaaacccctagacactgctaaatatagcctttgaacaggggatttgttgtattgttaacttgaaggtagagtcttatctcgttcctagaggggcctatagctcagtcggttagagcgcatcgctgataacgatgaggtcgcaagttcgattcttgctaggcccaccaggaacaaaaaaggggcattagctcagttggtagagcacctgctttgcaagcaggatgtcaggaggcgcgcctccacagttttcccggttcatcttgtttaaggtgggccgggatttttgttttttagtgtttctgggatacctcggtggcactccggcggcactctgggtcattggaaagctgtgtgtactttcgacaacaagatttgggcaaaatggtggtcaaaatcaggaaatcttgttgtcgaaagtacaactgggtcttcggttctatccgaggtgggtgagtagggtgcctgcaagtgcgatactggtgacaagtgtcgtggatccgaaagccagaatgaaaggcttgaccccaacatggatcaggttctggatttttacgccacacccgagaccaaacattgctgcagagagcaaggctgtctgtaggaaacctccagccgcaattacctcgtctgggagcgcaacagtggagcgcaaaactaccatcgccaagaatccaaggataaataggggaacgaccggtggtctctttccatcgggggacgtgtaaccctggcggcgctggcgaaaacttaaaatggcagcaatgggtgcaagtaggagtactcgggcgagtttcaccacaactgcaacacctaatgctccaccaccaatgactcctccagctgctactacttgggcgatttcatggatggatccgcctgcccacatcccaccgatttcaggggataaccccaagactttggttgcgaatgggataaaaggaatcatcagcgttccgaaaataacaacaagtgcaaccgcagtaacgacctcttcttcttcggaatcagttactccttcaacacctgccacggccgcagcgccacaaatagaaaagccacaagcaatcaacagaacttgggttggtttcattctgagcaggtgtcccatgaggatggtcccaaaaataccaccggcaacgatgcacacaatcaccgccagcatggggaaaccaagtgacaaaatatctgagaaaactaactgcagaccaaggaagacgatgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgg

SEQ ID NO: 55 - DNA, 10116 bp, synthetic construct
  misc_feature 1..10116: note = Vector Construct
  source 1..10116: mol_type = other DNA
cgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacggacggcgaattcctcctcgttgccacctccggcggctacggcaagcgcaccccacttgaggattactccacccagggccgtggtggcctcggcgtggtgaccttcaagtacaccccgaagcgcggtcgcctcgtcagcgccatcgcagttgaggaagatgacgagatcttcgccatcacctccgccggcggcgttgttcgcaccgaagtcaagcagatccgaccatcctcccgtgcaacaatgggtgttcgactggtcaacttggaagaaggtgtagaactgcttgccatcgacaagaacgtcgaagaccagggcgaagcatccgcagaagcagtagcaaagggtgcagtcgaaggaccagcatccaagactgctgccgaagaaaccgactccgttgacaacggatccgacgaaaacggcgaggaataatttatggcatcccgagaagtatccatcacccgaatctcaccactagcaaccttccgagttgcactggcaatgtccatcatcggactcgtggcgtggatcatctgcgtaaccgtcctctatttcggacttaacgtggctggcgtctggcagaacttcaacgatgtcatcggcggtgttggtgcagaacaaaccatcaccttcgggctcgtcctgagcatttccgcacttctgggagctatcggagcgattaccgtcgctgtgcttgcaccattgtgtgcaatcatctacaactcgattgttgacctcttcggtggactgcagattcaactgcaagaagaagtagactaacctctgaaacacaaacccctagacactgctaaatatagcctttgaacaggggatttgttgtattgttaacttgaaggtagagtcttatctcgttcctagaggggcctatagctcagtcggttagagcgcatcgctgataacgatgaggtcgcaagttcgattcttgctaggcccaccaggaacaaaaaaggggcattagctcagttggtagagcacctgctttgcaagcaggatgtcaggaggaccggtgttttagcgtgtcagtaggcgcgtagggtaagtggggtagcggcttgttagatatcttgaaatcggctttcaacagcattgatttcgatgtatttagctggccgttaccctgcgaatgtccacagggtagctggtagtttgaaaatcaacgccgttgcccttaggattcagtaactggcaaggaaggattttccaggaggaacacatgaacatcatcgctatcatgggccctcacggtgttttctacaaggatgagccaatcaaggagctggaatccgcactagttgcacagggctttcagatcatctggccccagaactccgttgaccttctcaaattcatcgagcacaatcctcgcatttgtggtgtgatttttgactgggacgagtactctcttgatttatgctccgacatcaaccagctcaacgagtacctgccactctacgcattcatcaacactcactccaccatggacgtttccgtgcaggacatgcgtatggcactctggttctttgaatacgctctgggccaggctgaggacatcgcgatccgcatgcgtcagtacaccgacgagtacctggacaacatcacccctccattcaccaaggctctcttcacctacgtaaaggaacgcaagtacactttctgcaccccaggccacatgggcggcaccgcctaccagaagtccccagtcggatgcctcttctacgacttcttcggcggtaacactcttaaggcagatgtctccatttccgtcaccgagttgggctctctgctggaccacaccggccctcacctggaggcagaagagtacatcgctcgtaccttcggtgctgaacagtcctacatcgtcaccaacggtacttccaccagcaacaagatcgttggtatgtacgcagctccttctggctccaccctgttgatcgaccgcaactgtcacaagtccctcgcgcatcttcttatgatgaacgatgtggtccctgtatggctgaagccaacccgtaacgctctgggcatccttggcggtatcccccgtcgcgagttcacccgtgattccatcgaggaaaaggttgcagccactacccaggcacagtggcctgtccacgctgtcattaccaactcgacctacgacggcctgctctacaacaccgattggatcaagcagaccctagatgttccttccattcacttcgacagcgcatgggttccttacactcacttccacccaatctaccagggtaagtccggaatgtccggcgagcgtgtcgctggcaaggttatcttcgaaacccaatcaacccacaagatgctggctgctctctcccaggcttctctgatccacatcaagggcgagtacgacgaggaagctttcaacgaggctttcatgatgcacaccaccacctccccatcctaccctatcgtcgcgtccgtcgagactgctgccgcaatgcttcgcggtaacccaggtaagcgcctcatcaaccgttccgttgagcgcgctcttcacttccgtaaggaagtgcagcgcctgcgtgaggaatctgacggttggttcttcgacatttggcagccacctcaggttgatgaggccgagtgctggccagttgctccaggtgaacagtggcacggattcaacgatgcagatgctgaccacatgtttttggacccggtcaaggtcaccattcttactcctggtatggatgagcagggcaacatgtctgaggagggtatcccagctgctctggttgcaaagttcctcgacgaacgtggcatcgttgttgagaagaccggaccatacaacctgctgttcctgttcagcatcggcatcgacaaaaccaaggcaatgggtctgctgcgcggccttaccgagttcaagcgctcctacgacctgaaccttcgcatcaagaatatgctgccggacctgtacgctgaagatcctgatttctaccgcaacatgcgcatccaggacctcgcacagggcatccacaagctcattcgcaagcacgacctgccaggccttatgctccgtgcattcgataccctcccagagatgatcatgacccctcaccaggcttggcagcgccagatcaagggcgaggtggaaaccatcgcactggagcagctggttggtcgtgtctccgccaacatgatcctgccatatccacctggcgttccgctgctgatgccaggcgagatgctcaccaaggagtcccgcaccgtattggacttccttctcatgttgtgctctgttggccagcactacccaggcttcgagaccgacatccacggcgctaagcaagatgaagacggcgtttaccgcgttcgcgtccttaagatggcaggctaacttgccagagcggcttccgggcgagtaacgtgcggatccgcctccacagttttcccggttcatcttgtttaaggtgggccgggatttttgttttttagtgtttctgggatacctcggtggcactccggcggcactctgggtcattggaaagctgtgtgtactttcgacaacaagatttgggcaaaatggtggtcaaaatcaggaaatcttgttgtcgaaagtacaactgggtcttcggttctatccgaggtgggtgagtagggtgcctgcaagtgcgatactggtgacaagtgtcgtggatccgaaagccagaatgaaaggcttgaccccaacatggatcaggttctggatttttacgccacacccgagaccaaacattgctgcagagagcaaggctgtctgtaggaaacctccagccgcaattacctcgtctgggagcgcaacagtggagcgcaaaactaccatcgccaagaatccaaggataaataggggaacgaccggtggtctctttccatcgggggacgtgtaaccctggcggcgctggcgaaaacttaaaatggcagcaatgggtgcaagtaggagtactcgggcgagtttcaccacaactgcaacacctaatgctccaccaccaatgactcctccagctgctactacttgggcgatttcatggatggatccgcctgcccacatcccaccgatttcaggggataaccccaagactttggttgcgaatgggataaaaggaatcatcagcgttccgaaaataacaacaagtgcaaccgcagtaacgacctcttcttcttcggaatcagttactccttcaacacctgccacggccgcagcgccacaaatagaaaagccacaagcaatcaacagaacttgggttggtttcattctgagcaggtgtcccatgaggatggtcccaaaaataccaccggcaacgatgcacacaatcaccgccagcatggggaaaccaagtgacaaaatatctgagaaaactaactgcagaccaaggaagacgatgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgg

SEQ ID NO: 56 - DNA, 48 bp, synthetic construct
  misc_feature 1..48: note = Primer
  source 1..48: mol_type = other DNA
acaggaaacagaattaattaagcttccccaccggaggacccccccatg

SEQ ID NO: 57 - DNA, 49 bp, synthetic construct
  misc_feature 1..49: note = Primer
  source 1..49: mol_type = other DNA
agtcgacctgcaggcatgcaagcttccggttggctaccggcctgccagg

SEQ ID NO: 58 - DNA, 101 bp, synthetic construct
  misc_feature 1..101: note = primer
  source 1..101: mol_type = other DNA
acaggaaacagaattaattaagctttgcaggaaggagatatagatatgcgctcgcacctgctcaacgacaccaccgcggagcactaccgccgctccgtgac

SEQ ID NO: 59 - DNA, 55 bp, synthetic construct
  misc_feature 1..55: note = primer
  source 1..55: mol_type = other DNA
agtcgacctgcaggcatgcaagcttttcgtgggtcagaactccgcgagggggttc

SEQ ID NO: 60 - DNA, 42 bp, synthetic construct
  misc_feature 1..42: note = Primer
  source 1..42: mol_type = other DNA
acagctatgacatgattacggacggcgaattcctcctcgttg

SEQ ID NO: 61 - DNA, 30 bp, synthetic construct
  misc_feature 1..30: note = Primer
  source 1..30: mol_type = other DNA
ggcgcgcctcctgacatcctgcttgcaaag

SEQ ID NO: 62 - DNA, 47 bp, synthetic construct
  misc_feature 1..47: note = Primer
  source 1..47: mol_type = other DNA
gcaggatgtcaggaggcgcgcctccacagttttcccggttcatcttg

SEQ ID NO: 63 - DNA, 50 bp, synthetic construct
  misc_feature 1..50: note = Primer
  source 1..50: mol_type = other DNA
atccccgggtaccgagctcgaattcatcgtcttccttggtctgcagttag

SEQ ID NO: 64 - DNA, 33 bp, synthetic construct
  misc_feature 1..33: note = Primer
  source 1..33: mol_type = other DNA
gcaagcaggatgtcaggaggaccggtgttttag

SEQ ID NO: 65 - DNA, 25 bp, synthetic construct
  misc_feature 1..25: note = Primer
  source 1..25: mol_type = other DNA
aaccgggaaaactgtggaggcggat

SEQ ID NO: 66 - DNA, 41 bp, synthetic construct
  misc_feature 1..41: note = Primer
  source 1..41: mol_type = other DNA
tcctagagccagtccgcgatcgcctccgcttcgacggtcac

SEQ ID NO: 67 - DNA, 43 bp, synthetic construct
  misc_feature 1..43: note = Primer
  source 1..43: mol_type = other DNA
attcgagctcggtacccgggtcaggcggaggcgccgcgagctg

SEQ ID NO: 68 - DNA, 831 bp, synthetic construct
  misc_feature 1..831: note = Vector Construct
  source 1..831: mol_type = other DNA
tcctagagccagtccgcgatcgcctccgcttcgacggtcacggccgttttgatgtagcacgcaactacgctatcgatgttacaggccgcggcgtgttcctccagaacgcaggcgttcacacccactccatcacctccccagacctcggcatgggcgcataccgcaatgccagcattatccgcgaactgctgggcaccgaatactacccagtcgagaaatccatcgcattccaagaattcgccgtctgatgcaggaaggagatatagatatgtccaccatcggtaccttcaccatccgccctcttgacccagagaaggacgcggagttgctgcaccgctgggtgacccacccaaaggccgcattctggatgatgcaggacgcacgcctagaagacgtagagcgcgaatacatgcgcatcgctgctcacgagcaccaccacgcctacctgggcctgcacgacggagagcctgctttcctcatggaaaaatacgacccacgccacgtcgaactggtgggcctgtacgaaccagaaccaggagacgttggcatgcacttcctcgtagcgccaaccgaccgcccagttcacggcttcacccgcgccgtcatcaccgccgttatggaagaactgttcgctgatcctgcgacccgccgcgtcgtcgttgagccagacgtcggcaacaaggctgtgcacgccctcaacgaagcagttggcttcgaacccgaacgcgaaatcgacaagccagagaagcgcgcactgctttccttctgcacccgcgaacagttcgaagcagctcgcggcgcctccgcctgacccgggtaccgagctcgaat

SEQ ID NO: 69 - DNA, 8498 bp, synthetic construct
  misc_feature 1..8498: note = Vector Construct
  source 1..8498: mol_type = other DNA
aattattcacacaggaaacagaattaattaccgtcctcgacctgatcgccggccacgccgtgcaggaaggagatatagatatgaccgctcttgctgacaccaccgagccaacctacgacttcgtcggcatcggtcttggacctttcaacctgggtctggcatgtctcaccgagccagtcgacgagctagcaggtgttttcctggaatccaagccagacttcgcttggcacgcgggaatgttcctggacggcgcccacctgcagaccccattcatgtccgacctggtcaccctggctgaccctaccagcccttacagctttctgaactaccttaaggagaagggacgtctttacagcttctacatccgcgaaaacttttacccactgcgtgtggaatacgacgattactgccgttgggcagctgcacagttatcctccatccgcttcggtaccacagtcaccgaagtcacctgggatgaaaccgctgaagtgtacgtcgtcgcgaccagcaccggtaccacgtaccgcggtcgtcatctggttctgggaaccggcaccgtcccgttcgtccctgaggcatgccgcggattggaaggcgaccttttccacaacgcacagtacatgcgtcgcaaggcagaactcctcgagaagaagtcggttaccgttgttggctccggccagtccgctgctgaaatctactacgagctcctttccggtatcgacgcacacggctaccaactaaactgggtcacccgctccccccgcttcttccctctcgaatacaccaagctcaccctcgaaatgacctcccccgactacattgattactttcgcgctctgcctgaagacacccgctaccgcctggaaaagcaacagaagggcctgttcaagggcatcaattcagacctcatcgacgcaatcttcgacctcctgtaccagaagaacgtcgaatccggtggccgcggcgtccccactcgcctgctcaccaactcctccctgaccaccgcccgctaccaggacggcacctacacacttggcttccgccaagacgaacagggaaaggatttcgaaatcgctactgagggcctggtcctcgccaccggctaccactacgaacagccagccttcctagagccagtccgcgatcgcctccgcttcgacggtcacggccgttttgatgtagcacgcaactacgctatcgatgttacaggccgcggcgtgttcctccagaacgcaggcgttcacacccactccatcacctccccagacctcggcatgggcgcataccgcaatgccagcattatccgcgaactgctgggcaccgaatactacccagtcgagaaatccatcgcattccaagaattcgccgtctgatgcaggaaggagatatagatatgtccaccatcggtaccttcaccatccgccctcttgacccagagaaggacgcggagttgctgcaccgctgggtgacccacccaaaggccgcattctggatgatgcaggacgcacgcctagaagacgtagagcgcgaatacatgcgcatcgctgctcacgagcaccaccacgcctacctgggcctgcacgacggagagcctgctttcctcatggaaaaatacgacccacgccacgtcgaactggtgggcctgtacgaaccagaaccaggagacgttggcatgcacttcctcgtagcgccaaccgaccgcccagttcacggcttcacccgcgccgtcatcaccgccgttatggaagaactgttcgctgatcctgcgacccgccgcgtcgtcgttgagccagacgtcggcaacaaggctgtgcacgccctcaacgaagcagttggcttcgaacccgaacgcgaaatcgacaagccagagaagcgcgcactgctttccttctgcacccgcgaacagttcgaagcagctcgcggcgcctccgcctgacccgggtaccgagctcgaattcagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagtccctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgagtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctcgattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttggcgtcccggaaaacgattccgaagcccaacctttcatagaaggcggcggtggaatcgaaatctcgtgatggcaggttgggcgtcgcttggtcggtcatttcgaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgtcgaagctcggcggatttgtcctactcaagctgatccgacaaaatccacacattatcccaggtgtccggatcggtcaaatacgctgccagctcatagaccgtatccaaagcatccggggctgatccccggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaatt

(19) United States
(12) Patent Application Publication
(10) Pub. No.: US 2025/0129397 A1
(43) Pub. Date: Apr. 24, 2025

(54) BIOTECHNOLOGICAL PRODUCTION OF MONOMERS OF BISUCABERINS, DESFERRIOXAMINES AND ANALOGS THEREOF

(71) Applicants: Evonik Operations GmbH, Essen (DE); Evoink Dr. Straetmans GmbH, Hamburg (DE)

(72) Inventors: Steffen SCHAFFER, Herten (DE); Mirja WESSEL, Bochum (DE); Alexander THIEMANN, Hasloh (DE); Hans Henning WENK, Mülheim an der Ruhr (DE)

(73) Assignees: Evonik Operations GmbH, Essen (DE); Evonik Dr. Straetmans GmbH, Hamburg (DE)

(21) Appl. No.: 18/866,187

(22) PCT Filed: May 11, 2023
(86) PCT No.: PCT/EP2023/062601

§ 371 Date: Nov. 15, 2024

(30) Foreign Application Priority Data
May 18, 2022 (EP) 22174070.7

(51) Int. Cl. C12P 13/02; C12N 9/00; C12N 9/02; C12N 9/10; C12N 9/88

(52) U.S. Cl. CPC C12P 13/02; C12N 9/0073; C12N 9/1025; C12N 9/88; C12N 9/93; C12Y 401/01018

(57) ABSTRACT

The present invention relates to a recombinant microbial cell for producing N5-aminopentyl-N-(hydroxy)-succinamic acid of Formula I from at least one simple carbon source:

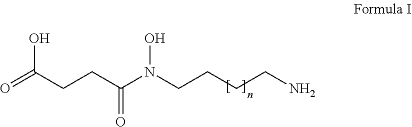

where n is 2 and
   wherein the simple carbon source is selected from the group consisting of glucose, sucrose, xylose, arabinose, mannose, glycerol and combinations thereof and wherein the cell comprises a further genetic modification to increase production of L-lysine from at least one of the simple carbon sources.

### FIELD OF THE INVENTION

The present invention relates to biotechnological production of monomers of bisucaberins, desferrioxamines and analogs thereof. In particular, the present invention relates to recombinant cells that are capable of biotechnological production of N5-aminopentyl-N-(hydroxy)-succinamic acid, N-hydroxy-N-succinyl putrescine and compounds therefrom.

### BACKGROUND OF THE INVENTION

Desferrioxamines, bisucaberins and other macrocyclic N-hydroxy-N-succinyl pentanediamine-based siderophores are well-known in several industries for their use in treatment of various diseases such as cancer, secondary iron overload and the like. These macrocyclic N-hydroxy-N-succinyl diamine-based siderophores are produced from monomers such as N-hydroxy-N-succinyl cadaverine, and N-hydroxy-N-succinyl putrescine by a dedicated biosynthetic pathway requiring four enzymatic activities and starting from L-lysine.

Macrocyclic N-hydroxy-N-succinyl diamine-based siderophores such as desferrioxamine B, H, E, X1, X2, bisucaberin, bisucaberin B and the like are currently produced by the bacterium Streptomyces pilosus (S. pilosus) and other Actinomycetes starting from lysine. In particular, these macrocyclic N-hydroxy-N-succinyl diamine-based siderophores and the monomers N-hydroxy-N-succinyl cadaverine, and N-hydroxy-N-succinyl putrescine from which these macrocyclic N-hydroxy-N-succinyl diamine-based siderophores are oligomerized and then macrocyclizised are produced by a dedicated biosynthetic pathway requiring four enzymatic activities and starting from L-lysine.

The macrocyclic N-hydroxy-N-succinyl diamine-based siderophores and the monomers N-hydroxy-N-succinyl cadaverine, and N-hydroxy-N-succinyl putrescine used predominantly in these pharmaceutical applications are today exclusively produced through fermentation using wildtype Streptomyces strains such as S. pilosus and S. parvulus. This method has a number of disadvantages outside of other problems.

Namely, the current method of production of has low production performance characteristics, such as biomass-specific productivity q_{P}, volumetric productivity Q_{P}, product yield on substrate Y_{X/S}, and product concentration. This results in high manufacturing costs. Further, since the macrocyclic N-hydroxy-N-succinyl diamine-based siderophores and the monomers N-hydroxy-N-succinyl cadaverine, and N-hydroxy-N-succinyl putrescine are formed using a simple fermentation process of wildtype Streptomyces strains, there will be a lot of byproducts in the fermentation broth produced. The Streptomyces species are potent producers of many secondary metabolites including antibiotics, which need to be separated from the desired target compounds by laborious and costly refinement steps. Also, the wild-type Streptomyces strains usually require complex and costly fermentation medium recipes due to complex growth requirements of the Streptomyces species, resulting in poor reproducibility due to batch-to-batch variation of complex medium components.

Further the use of lysine as the main carbon substrate of production also makes the process of forming macrocyclic N-hydroxy-N-succinyl diamine-based siderophores and the monomers N-hydroxy-N-succinyl cadaverine, and N-hydroxy-N-succinyl putrescine inflexible and costly.

While for pharmaceutical applications, high manufacturing costs may be acceptable, these costs are undesirable for other applications, such as in the cosmetic and technical (e.g. rust removal) field. Therefore, the transfer of biosynthesis of macrocyclic N-hydroxy-N-succinyl diamine-based siderophores and the monomers N-hydroxy-N-succinyl cadaverine, and N-hydroxy-N-succinyl putrescine to well-characterized microbial production strains, is highly desirable. Accordingly, there is a need in the art for a more efficient and affordable means of biosynthesis of macrocyclic N-hydroxy-N-succinyl diamine-based siderophores and the monomers N-hydroxy-N-succinyl cadaverine, and N-hydroxy-N-succinyl putrescine.

### DESCRIPTION OF THE INVENTION

The present invention attempts to solve the problems above by providing a biotechnological means of producing macrocyclic N-hydroxy-N-succinyl diamine-based siderophores and the monomers N-hydroxy-N-succinyl cadaverine, and N-hydroxy-N-succinyl putrescine thereof using an established microbial platform. Using an established microbial platform to produce at least one macrocyclic N-hydroxy-N-succinyl diamine-based siderophores and the monomers N-hydroxy-N-succinyl cadaverine, and N-hydroxy-N-succinyl putrescine not only increases the amount of target products produced from the starting material but also reduces the amount of byproducts formed. Also, the genetically modified cell according to any aspect of the present invention has the advantage of being non-pathogenic and simple to culture. This enables the cell to be safer for production and also keeps the costs lower as no special safety requirements are needed in the lab during production and use of the macrocyclic N-hydroxy-N-succinyl diamine-based siderophores and the monomers N-hydroxy-N-succinyl cadaverine, and N-hydroxy-N-succinyl putrescine thereof. The efficiency of production of macrocyclic N-hydroxy-N-succinyl diamine-based siderophores and the monomers N-hydroxy-N-succinyl cadaverine, and N-hydroxy-N-succinyl putrescine is also increased with the use of a recombinant cell according to any aspect of the present invention. Also, the use of microbial platforms capable of integrating the entire means of converting a carbon source to at least one macrocyclic N-hydroxy-N-succinyl diamine-based siderophores and the monomers N-hydroxy-N-succinyl cadaverine, and N-hydroxy-N-succinyl putrescine thereof, makes the process of conversion simpler as only a small number of process steps are involved in the conversion. The reliance of Streptomyces strains for production of macrocyclic N-hydroxy-N-succinyl diamine-based siderophores and the monomers N-hydroxy-N-succinyl cadaverine, and N-hydroxy-N-succinyl putrescine is also removed. The cells according to any aspect of the present invention has the further advantage of being able to use a variety of carbon substrates to produce the macrocyclic N-hydroxy-N-succinyl diamine-based siderophores and the monomers N-hydroxy-N-succinyl cadaverine, and N-hydroxy-N-succinyl putrescine according to any aspect of the present invention. For examples simple carbons such as glucose may be used as a carbon substrate.

The cells used according to any aspect of the present invention, results in several advantages including:
Higher production performance characteristics, such as biomass-specific productivity q_{P}. volumetric productivity Q_{P}, product yield on substrate Y_{X/s}, and product concentration, resulting in lower manufacturing costs;
Lower diversity and/or concentration or absence of by-products in the fermentation broth simplifying refinement strategies, resulting in lower manufacturing cost; and
Applicability of defined minimal fermentation media resulting in lower manufacturing costs and better reproducibility.

According to one aspect of the present invention, there is provided a recombinant microbial cell for producing at least one compound of Formula I from at least one simple carbon source:

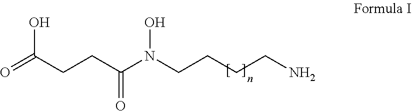

where n is 1 or 2
   wherein the simple carbon source is selected from the group consisting of glucose, sucrose, xylose, arabinose, mannose, glycerol and combinations thereof and wherein the cell comprises a further genetic modification to increase production of L-lysine from at least one of the simple carbon sources.

In one example, n is 2 and the compound produced is N-hydroxy-N-succinyl cadaverine, also known as N5-aminopentyl-N-(hydroxy)-succinamic acid. In another example, n is 2 and the compound produced is N-hydroxy-N-succinyl putrescine. In another example, both compounds N5-aminopentyl-N-(hydroxy)-succinamic acid and N-hydroxy-N-succinyl putrescine are produced in a mixture according by the cell according to any aspect of the present invention.

In particular, n is 2 and the compound produced is N-hydroxy-N-succinyl cadaverine, also known as N5-aminopentyl-N-(hydroxy)-succinamic acid.

The term ‘recombinant’ as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild-type cell that comprises the original unaltered nucleic acid molecule. Furthermore, the term “recombinant DNA” refers to a nucleic acid sequence which is not naturally occurring or has been made by the artificial combination of two otherwise separated segments of nucleic acid sequence, i.e., by ligating together pieces of DNA that are not normally contiguous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al, Current Protocols in Molecular Biology, Current Protocols (1989), and DNA Cloning: A Practical Approach, Volumes I and II (ed. D. N. Glover) IREL Press, Oxford, (1985). In another example, a cell is recombinant if the cell has been modified, particularly has undergone genetic engineering and is a non-naturally occurring cell. The term “recombinant cell” used herein refers to a cell that has been genetically modified to comprise at least one heterologous gene encoding at least one heterologous protein, for example, enzyme. The recombinant cell may express the heterologous protein. The protein may participate in a metabolic pathway for production of a desirable metabolite. In another example, the ‘recombinant cell’ refers a cell that already expresses a specific enzyme(s) and the expression of the enzyme is modified using genetic engineering. In this example, endogenously expressed genes are genetically modified to increase or decrease the expression of the gene using methods known in the art. Exemplary cells include prokaryotic cells and eukaryotic cells. Exemplary prokaryotic cells include bacteria, such as C. glutamicum, such as genetically modified C. glutamicum.

The recombinant microbial cell used according to any aspect of the present invention may be prokaryotes or eukaryotes. These cells are isolated cells. These can be mammalian cells (such as, for example, cells from man), plant cells or microorganisms such as yeasts, fungi or bacteria, wherein microorganisms in particular bacteria and yeasts are preferred.

Suitable bacteria, yeasts or fungi are in particular those bacteria, yeasts or fungi that are deposited in the Deutsche Sammlung von Mikroorganismen und Zellkulturen (German Collection of Microorganisms and Cell Cultures) GmbH (DSMZ), Brunswick, Germany, as bacterial, yeast or fungal strains. Bacteria suitable according to the invention belong to the genera that are listed under:
http://www.dsmz.de/species/bacteria.htm,
   yeasts suitable according to the invention belong to those genera that are listed under:
http://www.dsmz.de/species/yeasts.htm
   and fungi suitable according to the invention are those that are listed under:
http://www.dsmz.de/species/fungi.htm.

In particular, the cells may be selected from the genera Aspergillus, Corynebacterium, Brevibacterium, Bacillus, Acinetobacter, Alcaligenes, Lactobacillus, Paracoccus, Lactococcus, Candida, Pichia, Hansenula, Kluyveromyces, Saccharomyces, Escherichia, Zymomonas, Yarrowia, Methylobacterium, Ralstonia, Pseudomonas, Rhodospirillum, Rhodobacter, Burkholderia, Clostridium and Cupriavidus. More in particular, the cells may be selected from the group consisting of Aspergillus nidulans, Aspergillus niger, Alcaligenes latus, Bacillus megaterium, Bacillus subtilis, Brevibacterium flavum, Brevibacterium lactofermentum, Burkholderia andropogonis, B. brasilensis, B. caledonica, B. caribensis, B. caryophylli, B. fungorum, B. gladioli, B. glathei, B. glumae, B. graminis, B. hospita, B. kururiensis, B. phenazinium, B. phymatum, B. phytofirmans, B. plantarii, B. sacchari, B. singaporensis, B. sordidicola, B. terricola, B. tropica, B. tuberum, B. ubonensis, B. unamae, B. xenovorans, B. anthina, B. pyrrocinia, B. thailandensis, Candida blankii, Candida rugosa, Corynebacterium glutamicum, Corynebacterium efficiens, Escherichia coli, Hansenula polymorpha, Kluveromyces lactis, Methylobacterium extorquens, Paracoccus versutus, Pseudomonas argentinensis, P. borbori, P. citronellolis, P. flavescens, P. mendocina, P. nitroreducens, P. oleovorans, P. pseudoalcaligenes, P. resinovorans, P. straminea, P. aurantiaca, P. aureofaciens, P. chlororaphis, P. fragi, P. lundensis, P. taetrolens, P. antarctica, P. azotoformans, ‘P. blatchfordae’, P. brassicacearum, P. brenneri, P. cedrina, P. corrugata, P. fluorescens, P. gessardii, P. libanensis, P. mandelii, P. marginalis, P. mediterranea, P. meridiana, P. migulae, P. mucidolens, P. orientalis, P. panacis, P. proteolytica, P. rhodesiae, P. synxantha, P. thivervalensis, P. tolaasii, P. veronii, P. denitrificans, P. pertucinogena, P. cremoricolorata, P. fulva, P. monteilii, P. mosselii, P. parafulva, P. putida, P. balearica, P. stutzeri, P. amygdali, P. avellanae, P. caricapapayae, P. cichorii, P. coronafaciens, P. ficuserectae, ‘P. helianthi’, P. meliae, P. savastanoi, P. syringae, P. tomato, P. viridiflava, P. abietaniphila, P. acidophila, P. agarici, P. alcaliphila, P. alkanolytica, P. amyloderamosa, P. asplenii, P. azotifigens, P. cannabina, P. coenobios, P. congelans, P. costantinii, P. cruciviae, P. delhiensis, P. excibis, P. extremorientalis, P. frederiksbergensis, P. fuscovaginae, P. gelidicola, P. grimontii, P. indica, P. jessenii, P. jinjuensis, P. kilonensis, P. knackmussii, P. koreensis, P. lini, P. lutea, P. moraviensis, P. otitidis, P. pachastrellae, P. palleroniana, P. papaveris, P. peli, P. perolens, P. poae, P. pohangensis, P. psychrophila, P. psychrotolerans, P. rathonis, P. reptilivora, P. resiniphila, P. rhizosphaerae, P. rubescens, P. salomonii, P. segitis, P. septica, P. simiae, P. suis, P. thermotolerans, P. aeruginosa, P. tremae, P. trivialis, P. turbinellae, P. tuticorinensis, P. umsongensis, P. vancouverensis, P. vranovensis, P. xanthomarina, Ralstonia eutropha, Rhodospirillum rubrum, Rhodobacter sphaeroides, Saccharomyces cerevisiae, Vibrio natrigens, Yarrowia lipolytica and Zymomonas mobile. More in particular, the cell may be a bacterial cell selected from the genera Pseudomonas, Cyanobacteria Corynebacterium, Brevibacterium, Bacillus, Klebsiella, Salmonella, Rhizobium, Vibrio, Saccharomyces, Yarrowia, Aspergillus, Trichoderma, Chlorella, Nostoc and Escherichia.

Even more in particular, the cells may be selected from the group consisting of Pseudomonas putida, Escherichia coli, Burkholderia thailandensis, Pseudomonas fluorescens, Pseudomonas putida, Pseudomonas stutzeri, Burkholderia thailandensis, Klebsiella oxytoca, Rhizobium meliloti, Bacillus subtilis, Vibrio natrigens, and Corynebacterium glutamicum. More in particular, the cell may be C. glutamicum or Escherichia coli.

The terms “foreign”, “exogenous”, and “heterologous” are used herein interchangeably and refers to a molecule, for example, a polynucleotide (e.g., gene), a protein (e.g., enzyme), or a metabolite produced or expressed in a cell from a microorganism with genetic modification (i.e., recombinant cell) but not in a cell from the microorganism without any generic modifications (i.e. the wild-type cell).

The phrase “wild-type” as used herein in conjunction with a cell or microorganism may denote a cell with a genome make-up that is in a form as seen naturally in the wild. The term may be applicable for both the whole cell and for individual genes. The term ‘wild-type’ may thus also include cells which have been genetically modified in other aspects (i.e. with regard to one or more genes) but not in relation to the genes of interest. The term “wild-type” therefore does not include such cells where the gene sequences of the specific genes of interest have been altered at least partially by man using recombinant methods. A wild-type cell according to any aspect of the present invention thus refers to a cell that has no genetic mutation with respect to the whole genome and/or a particular gene. Therefore, in one example, a wild-type cell with respect to enzyme E₁ may refer to a cell that has the natural/non-altered expression of the enzyme E₁ in the cell. The wild-type cell with respect to enzyme E₂, E₃, E₄, E₅, etc. may be interpreted the same way and may refer to a cell that has the natural/non-altered expression of the enzyme E₂, E₃, E₄, E₅, etc. respectively in the cell.

The terms “natural”, “native”, “endogenous” and “homologous” are used interchangeably and refers to a molecule, for example, a polynucleotide (e.g., gene), a protein (e.g., enzyme), or a metabolite produced or expressed a cell from a microorganism without any generic modification.

The terms “production” and “expression” are used herein interchangeably and refer to transcription of a gene and/or translation of an mRNA transcript into a protein by a cell.

The term “feedstock” as used herein refers to the nutrients supplied to a recombinant cell in a culture medium for production of a desirable molecule (e.g., metabolite). For example, a carbon source such as a biomass or a carbon compound derived from a biomass is a feedstock for a microorganism in a fermentation process or in other growth contexts, such as a live vaccine vector or immunotherapy. The feedstock may contain nutrients as well as sources of energy.

The term “carbon source” as used herein refers to a substance suitable for use as a source of carbon, for the recombinant cell according to any aspect of the present invention to produce bisucaberins and/or analogs thereof. In other words, the carbon source is considered the starting material for the formation of desferrioxamine and/or an analog thereof. Carbon sources include, but are not limited to, glucose, biomass hydrolysates, starch, sucrose, cellulose, hemicellulose, xylose, lignin and monomer components of these substrates. Without being limitative, carbon sources may include various organic compounds in various forms including polymers, carbohydrates, acids, alcohols, aldehydes, ketones, amino acids and peptides. Examples of these include various monosaccharides, for example, glucose, dextrose (D-glucose), maltose, oligosaccharides, polysaccharides, saturated or unsaturated fatty acids, succinic acid, lactic acid, acetic acid, ethanol, rice bran, molasses, corn decomposition solution, cellulose decomposition solution, and mixtures of the foregoing. In particular, the carbon source may be selected from the group consisting of glucose, sucrose, xylose, arabinose, mannose, lysine and cadaverine. More in particular, the carbon source used according to any aspect of the present invention may be a simple carbon source. The term “simple carbon source” is understood to mean carbon sources wherein in the carbon skeleton at least one C—C bond has been broken. In particular, the simple carbon source may be at least one carbohydrate such as for example glucose, saccharose, arabinose, xylose, lactose, fructose, maltose, molasses, starch, cellulose, glycerine and hemicellulose, but carbon sources may also include glycerin or very simple organic molecules such as CO2, CO or synthesis gas.

The term “substrate” used herein refers to a compound that is converted to another compound by the action of one or more enzymes, or that is intended for such conversion. The term includes not only a single type of compound but also any combination of compounds, such as a solution, mixture or other substance containing at least one substrate or its derivative. Furthermore, the term “substrate” includes not only compounds that provide a carbon source suitable for use as a starting material such as sugar, derived from a biomass, but also intermediate and final product metabolites used in pathways associated with the metabolically manipulated microorganisms described in the present specification.

The terms “polynucleotide” and “nucleic acid” are used herein interchangeably and refer to an organic polymer comprising two or more monomers including nucleotides, nucleosides or their analogs, and include, but are not limited to, single-stranded or double-stranded sense or antisense deoxyribonucleic acid (DNA) of arbitrary length, and where appropriate, single-stranded or double-stranded sense or antisense ribonucleic acid (RNA) of arbitrary length, including siRNA.

The terms “protein” and “polypeptide” are used herein interchangeably and refer to an organic polymer composed of two or more amino acid monomers and/or analog and joined together by peptide bonds between the carboxyl and amino groups of adjacent amino acid residues.

The terms “amino acid” and “amino acid monomer” are used herein interchangeably and refer to a natural or synthetic amino acid, for example, glycine and both D- or L-optical isomers. The term “amino acid analog” as used herein refers to an amino acid wherein one or more individual atoms has been replaced with different atoms or different functional groups.

Any of the enzymes used according to any aspect of the present invention, may be an isolated enzyme. In particular, the enzymes used according to any aspect of the present invention may be used in an active state and in the presence of all cofactors, substrates, auxiliary and/or activating polypeptides or factors essential for its activity. The term “isolated”, as used herein, means that the enzyme of interest is enriched compared to the cell in which it occurs naturally. The enzyme may be enriched by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye. The enzyme used according to any aspect of the present invention may be recombinant.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least once or twice, especially at least 10 times, at least 100 times, at least 1000 times or at least 10000 times more bisucaberins and/or analogs thereof than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (bisucaberins and/or analogs thereof) in the nutrient medium.

The genetically modified cell or microorganism may be genetically different from the wild-type cell or microorganism. The genetic difference between the genetically modified microorganism according to any aspect of the present invention and the wild-type microorganism may be in the presence of a complete gene, amino acid, nucleotide etc. in the genetically modified microorganism that may be absent in the wild-type microorganism. In one example, the genetically modified microorganism according to any aspect of the present invention may comprise enzymes that enable the microorganism to produce more bisucaberins and/or analogs thereof compared to the wild-type cells. The wild-type microorganism relative to the genetically modified microorganism of the present invention may have none or no detectable activity of the enzymes that enable the genetically modified microorganism to produce bisucaberins and/or analogs thereof. As used herein, the term ‘genetically modified microorganism’ may be used interchangeably with the term ‘genetically modified cell’. The genetic modification according to any aspect of the present invention is carried out on the cell of the microorganism.

The cells according to any aspect of the present invention are genetically transformed according to any method known in the art. In particular, the cells may be produced according to the method disclosed in WO2013024114.

The phrase ‘the genetically modified cell has an increased activity, in comparison with its wild-type, in enzymes’ as used herein refers to the activity of the respective enzyme that is increased by a factor of at least 2, in particular of at least 10, more in particular of at least 100, yet more in particular of at least 1000 and even more in particular of at least 10000.

The phrase “increased activity and/or or expression of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity, altering the codon utilization of the gene, increasing the half-life of the mRNA or of the enzyme in various ways, modifying the regulation of the expression of the gene and optionally by combining these measures. Genetically modified cells used according to any aspect of the present invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. In particular, activity or expression of an enzyme may be increased or enhanced in a cell by a method selected from the group consisting of
a) introducing a promoter or promoters which are operably linked to the gene encoding the enzymes into the chromosome of said cell,
b) increasing the copy number of the genes encoding the enzymes by introducing one or more expression vectors into said cell, and
c) combinations thereof.

In particular, the cell according to any aspect of the present invention comprises a genetic modification of
a) at least one promoter which is operably linked to gene(s) encoding the any one of the enzymes in a suitable chromosome of the cell, or
b) at least one expression vector in the cell to increase the copy number of gene(s) encoding the enzymes, or
c) combination of (a) and (b)
to increase the expression of activity of the enzymes.

In one example, the enzymes are E₁, E₂ and E₃. Even more in particular, the cell according to any aspect of the present invention comprises a genetic modification of
a) at least one promoter which is operably linked to gene(s) encoding the any one of the enzymes E₁, E₂ and E₃ in a suitable chromosome of the cell, or
b) at least one expression vector in the cell to increase the copy number of gene(s) encoding the enzymes E₁, E₂ and E₃, or
c) combination of (a) and (b)
   to increase the expression of the enzymes E₁, E₂ and E₃ in the cell according to any aspect of the present invention.

In another example, the cell according to any aspect of the present invention may comprise a further genetic modification to comprise
a) at least one promoter which is operably linked to a gene encoding any one of the enzymes E₆-E₁₄, and E₁₇-E₁₉ in the suitable chromosome of the cell, or
b) at least one expression vector in the cell to increase the copy number of gene(s) encoding any one of the enzymes E₆-E₁₄, and E₁₇-E₁₉, or
c) combination of (a) and (b)
   to increase the activity of any one of the enzymes E₆-E₁₄, and E₁₇-E₁₉ in the cell and/or
d) a foreign DNA in the gene encoding at least one of enzymes E₁₅ and E₁₆;
e) deletion of at least one part of the gene encoding at least one of enzymes E₁₅ and E₁₆;
f) at least one point mutation, RNA interference (siRNA), antisense RNA in the gene and/or regulatory sequences of the gene encoding at least one of enzymes E₁₅ and E₁₆; or
g) combinations of (d), (e) and/or (f)
   to decrease the activity of at least one of the enzymes E₁₅ and E₁₆ in the cell.

In one example, the cell according to any aspect of the present invention comprises a genetic modification that results in an increased activity of at least enzymes E₂, E₃, and comprises a further genetic modification to increase production of Lysine. In this example, increased lysine production may be due to the cell have a further genetic modification that increases activity of at least one enzyme selected from the group consisting of E₆-E₁₄, and E₁₇-E₁₉ and/or decreases activity of at least one enzyme selected from E₁₅ and E₁₆. In this context, the term ‘suitable chromosome’ refers to the original chromosome to which the gene which codes for enzymes E₁, E₂, and/or E₃ is found. Therefore, the suitable chromosome is the source of the chromosome from which the gene originates.

In the same context, the phrase “decreased activity and/or expression of an enzyme Eₓ” used with reference to any aspect of the present invention may be understood as meaning an activity decreased by a factor of at least 0.5, particularly of at least 0.1, more particularly of at least 0.01, even more particularly of at least 0.001 and most particularly of at least 0.0001. The phrase “decreased activity” also comprises no detectable activity (“activity of zero”). The decrease in the activity of a certain enzyme can be effected, for example, by selective mutation or by other measures known to the person skilled in the art for decreasing the activity of a certain enzyme. In particular, the person skilled in the art finds instructions for the modification and decrease of protein expression and concomitant lowering of enzyme activity by means of interrupting specific genes, for example at least in Dubeau et al. 2009. Singh & Röhm. 2008., Lee et al., 2009 and the like. The decrease in the enzymatic activity in a cell according to any aspect of the present invention may be achieved by modification of a gene comprising one of the nucleic acid sequences, wherein the modification is selected from the group comprising, consisting of, insertion of foreign DNA in the gene, deletion of at least parts of the gene, point mutations in the gene sequence, RNA interference (siRNA), antisense RNA or modification (insertion, deletion or point mutations) of regulatory sequences, such as, for example, promoters and terminators or of ribosome binding sites, which flank the gene. In particular, to decrease the activity of an enzyme in a cell, the cell may comprise
d) a foreign DNA in the gene encoding the enzyme;
e) a deletion of at least one part of the gene encoding the enzyme;
f) at least one point mutation, RNA interference (siRNA), antisense RNA in the gene and/or regulatory sequences of the gene encoding the enzyme; or
g) combinations of (d), (e) and (f)

The expression of the enzymes and genes mentioned above, and all mentioned below is determinable by means of 1- and 2-dimensional protein gel separation followed by optical identification of the protein concentration in the gel with appropriate evaluation software.

If the increasing of an enzyme activity is based exclusively on increasing the expression of the corresponding gene, then the quantification of the increasing of the enzyme activity can be simply determined by a comparison of the 1- or 2-dimensional protein separations between wild-type and genetically modified cell. A common method for the preparation of the protein gels with bacteria and for identification of the proteins is the procedure described by Hermann et al. (Electrophoresis, 22: 1712-23 (2001). The protein concentration can also be analysed by Western blot hybridization with an antibody specific for the protein to be determined (Sambrook et al., Molecular Cloning: a laboratory manual, 2nd Ed. Cold Spring Harbor Laboratory Press, Cold Spring Harbor, N.Y. USA, 1989) followed by optical evaluation with appropriate software for concentration determination (Lohaus and Meyer (1989) Biospektrum, 5: 32-39; Lottspeich (1999), Angewandte Chemie 111: 2630-2647). This method is also always an option when possible products of the reaction to be catalysed by the enzyme activity to be determined may be rapidly metabolized in the microorganism or else the activity in the wild-type is itself too low for it to be possible adequately to determine the enzyme activity to be determined on the basis of the production formation.

The cell according to any aspect of the present invention is genetically modified or may comprise a genetic modification that increases activity relative to its wild-type cell of at least two enzymes selected from E₁, E₂, and E₃, wherein:
E₁ is a lysine decarboxylase (EC: 4.1.1.18) capable of converting lysine to cadaverine;
E₂ is a cadaverine N5-monooxygenase (EC 1.14.13.-) capable of converting cadaverine to N5-hydroxy-cadaverine; and
E₃ is a N5-Aminopentyl-N-(hydroxy)-succinamic acid synthase (EC: 2.3.-.-) capable of converting N5-hydroxy-cadaverine and succinyl-coenzyme A to N5-aminopentyl-N-(hydroxy)-succinamic acid or N-hydroxy-N-succinyl putrescine.

In one example, the cell according to any aspect of the present invention may comprise a genetic modification that increases activity relative to its wild-type cell of at least E₂, and E₃. In another example, the cell according to any aspect of the present invention may comprise a genetic modification that increases activity relative to its wild-type cell of E₁, E₂, and E₃.Lysine decarboxylase (E₁) is capable of converting lysine to cadaverine. In particular, E₁ converts lysine to cadaverine also known as 1,5-pentanediamine. Suitable polynucleotides which code for lysine decarboxylase (E₁) may be obtained from strains of, for example, Escherichia coli, Bacillus halodurans, Bacillus cereus, Bacillus subtilis, Bacillus thuringensis, Burkholderia ambifaria, Burkholderia vietnamensia, Burkholderia cenocepatia, Chromobacterium violaceum, Corynebacterium xerosis, Selenomonas ruminantium, Vibrio cholerae, Vibrio parahaemolyticus, Streptomyces coelicolor, Streptomyces violaceoruber, Streptomyces pilosus, Eikenalla corrodens, Eubacterium acidaminophilum, Francisella tulariensis, Geobacillus kaustophilus, Salmonella typhi, Salmonella typhimurium, Hafnia alvei, Neisseria meningitidis, Thermoplasma acidophilum, Plasmodium falciparum, Kineococcus radiotolerans, Oceanobacillus iheyensis, Pyrococcus abyssi, Porochlorococcus marinus, Proteus vulgaris, Rhodoferax ferrireducens, Saccharophagus degradans, Streptococcus pneumoniae, Synechococcus sp. The amino acid sequences of these lysine decarboxylases are registered in a database (GenBank).

Suitable lysine decarboxylases which can be employed in the process according to any aspect of the present invention are understood to be enzymes and their alleles or mutants which are capable of decarboxylating lysine, particularly L-lysine.

In particular, the lysine decarboxylase (E₁) is selected from the group consisting of E. coli, Corynebacterium xerosis, Streptomyces coelicolor, Streptomyces violaceoruber and Streptomyces pilosus from whose safety has been confirmed. More in particular, E₁ may be selected from the group consisting of Streptomyces violaceoruber, Streptomyces pilosus and E. coli. Even more in particular, E₁ comprises at least 70% sequence identity relative to SEQ ID NO:15 (E₁ₐ), SEQ ID NO:25 (E_{1b}) or SEQ ID NO:50 (E_{1c}).SEQ ID NO:. These sequences are available freely in internationally accessible databases such as, for example, that of the National Library of Medicine and the National Institute of Health (NIH) of the United States of America. The same sequence is also available freely at the Institute Pasteur (France) on the colibri web server under the gene name cadA. The same sequence is also available free through the web server ExPasy, which is maintained by the Swiss Institute of Bioinformatics, under the gene name cadA.

E₂ is a cadaverine N5-monooxygenase capable of converting cadaverine to N5-hydroxy-cadaverine. In particular, E₂ converts cadaverine to N5-hydroxy-cadaverine. E₂ may also be called DesB, or lysine N(6)-hydroxylase/L-ornithine N(5)-oxygenase family protein or L-lysine 6-monooxygenase (dfoA). More in particular, E₂ may be from Streptomyces leeuwenhoekii (CQR63915.1 GI:822880125), Streptomyces venezuelae ATCC 10712 (CCA55857.1 GI:328882618), Streptomyces fulvissimus DSM 40593 (AGK77332.1 GI:485094149), Streptomyces scabiei 87.22 (CBG72818.1 GI:260649703), Streptomyces sp. PAMC 26508 (AGJ55094.1 GI:478746514), Streptomyces coelicolor A3(2) (CAB87220.1 GI:7544047), Streptomyces coelicolor CAI-140, Streptomyces ambofaciens ATCC 23877 (AKZ55867.1 GI:917649198), Streptomyces ambofaciens (CAL18383.1 GI:115501977), Streptomyces sp. (QTK16919.1 GI:2021361636), Streptomyces sp. OM5714 (KAF2779951.1 GI:1812043702), Streptomyces sp. CBMAI 2042 (RLV66814.1 GI:1495247117), Nocardiopsis sp. JB363 (SIO88001.1 GI:1143070536), Streptomyces venezuelae (ALO08592.1 GI:952470254), Streptomyces venezuelae (CUM41036.1 GI:932870025), Streptomyces formicae (ATL27940.1 GI:1259310059), Streptomyces violaceoruber, Streptomyces pilosus, Orrella dioscoreae (SOE50134.1 GI:1253556185), Orrella dioscoreae (SBT27311.1 GI:1037119819), Streptomyces sp. NL15-2K (GCB49137.1 GI:1493708164), Micromonospora sp. B006 (AXO35035.1 GI:1450456451), Streptomyces bottropensis ATCC 25435 (EMF50428.1 GI:456384850), Actinobacteria bacterium OV450 (KPI33963.1 GI:930403533), Micromonospora saelicesensis (RAN96846.1 GI:1408858225) or a plant metagenome with accession number VGO10077.1 GI:1613564795, VFR50302.1 GI:1591769534, VFR49623.1 GI:1591759477, VFR70778.1 GI:1591736834, VFR85324.1 GI:1591730130, VFR90816.1 GI:1591727176, VFR74009.1 GI:1591713764, VFR35744.1 GI:1591707917, VFR18051.1 GI:1591706292, VFR25000.1 GI:1591690076, or VFR75029.1 GI:1591683182.

E₂ may also be called Siderophore biosynthesis protein, monooxygenase and may also be selected from Corynebacterium xerosis (SLM95113.1), Brachybacterium tyrofermentans (WP_193116041.1), Brachybacterium ginsengisoli (WP_096799042.1), Brachybacterium sp. HMSC06H03 (WP_070499498.1), Actinomycetales bacterium (NMA75681.1), Candidatus Brachybacterium intestinipullorum (HJC70675.1), Brachybacterium sp. AG952 (WP_133677657.1), Brachybacterium paraconglomeratum (WP_010552292.1), Brachybacterium sp. SW0106-09 (WP_053916852.1), Brachybacterium sp. Sponge (WP_062950357.1), Brachybacterium sp. SGAir0954 (WP_137770520.1), Brachybacterium phenoliresistens (WP_038369982.1), Brachybacterium subflavum (WP_152352729.1), Brachybacterium endophyticum (WP_109276394.1), Brachybacterium sacelli (WP_209904120.1), Brachybacterium halotolerans (WP_200500938.1), Brachybacterium sp. CBA3105 (WP_228358231.1), Brachybacterium sp. P6-10-X1 (WP_083713362.1), Brachybacterium sp. P6-10-X1 (APX34711.1), Brachybacterium sp. FME24 (WP_193105844.1), Brachybacterium sp. YJGR34 (WP_114855299.1), Isoptericola Cucumis (WP_188524545.1), Microbacterium marinilacus (WP_221859754.1), Agreia bicolorata (WP_044440800.1), Isoptericola variabilis (WP_144881770.1), Isoptericola variabilis (WP_013837804.1), Xylanimonas oleitrophica (WP_111251928.1), Brevibacterium (WP_113902445.1), Antribacter sp. KLBMP9083 (WP_236088632.1), Isoptericola (WP_106267363.1), Xylanimonas cellulosilytica (WP_012878564.1), Isoptericola chiayiensis (WP_172152929.1), Promicromonospora citrea (WP_171102616.1), Isoptericola jiangsuensis (WP_098462726.1), Brevibacterium renqingii (WP_209323545.1), Brevibacterium renqingii (WP_209371577.1), Brevibacterium sp. W7.2 (WP_211978854.1), Brevibacterium casei (WP_198499439.1), Brevibacterium sandarakinum (WP_092106697.1), Brevibacterium linens (WP_052239998.1), Brevibacterium sp. RIT 803 (WP_204232889.1), Brevibacterium sp. VCM10 (WP_025776857.1), Brevibacterium iodinum (WP_101543359.1), Brevibacterium (WP_137827005.1), Brevibacterium sp. CFH 10365 (WP_152347365.1), Brevibacterium pigmentatum (WP_167198402.1), Brevibacterium siliguriense (WP_092011613.1), Brevibacterium limosum (WP_166822890.1), Brevibacterium sediminis (WP_181272454.1), Brevibacterium aurantiacum (WP_096178869.1), Brevibacterium aurantiacum (WP_114384950.1), Brevibacterium sp. UCMA 11754 (WP_235346449.1), Brevibacterium aurantiacum (WP_143924264.1), Brevibacterium antiquum (WP_198396798.1), Brevibacterium sp. CCUG 69071 (WP_230744765.1), Brevibacterium marinum (WP_167950433.1), Brevibacterium sp. HY170 (WP_231442298.1), Brevibacterium sp. S22 (WP_135810285.1), Brevibacterium aurantiacum (WP_193102124.1), Brevibacterium aurantiacum (WP_193086562.1), Brevibacterium aurantiacum (WP_101639104.1), Brevibacterium aurantiacum (WP_125178290.1), Brevibacterium aurantiacum (WP_135446719.1), Brevibacterium epidermidis (WP_098731558.1), Brevibacterium linens (WP_062862175.1), Brevibacterium epidermidis (WP_062243347.1), Brevibacterium linens (WP_139908726.1), Brevibacterium epidermidis (HJE77366.1), Brevibacterium aurantiacum (WP_096157398.1), Brevibacterium aurantiacum (WP_193084517.1), Brevibacterium aurantiacum (WP_101584429.1), Brevibacterium aurantiacum (WP_096167625.1), Brevibacterium aurantiacum (WP_096145959.1), Brevibacterium aurantiacum (WP_096160423.1), Brevibacterium aurantiacum (WP_069600710.1), Brevibacterium aurantiacum (WP_009883551.1), Brevibacterium sp. UCMA 11752 (WP_235350997.1), Brevibacterium sp. MG-1 (WP_139468396.1), Candidatus Brevibacterium intestinavium (HJA61508.1), Brevibacterium sp. SMBL_HHYL_HB1 (WP_212129584.1), Brevibacterium atlanticum (WP_166973261.1), Brevibacterium aurantiacum (WP_193078848.1), Brevibacterium aurantiacum (WP_096163051.1), Brevibacterium sp. CT2-23B (WP_172170764.1), Brevibacterium linens (HJF75769.1), Brevibacterium aurantiacum (WP_101598305.1), Brevibacterium sp. ‘Marine’ (WP_169252964.1), Brevibacterium sp. S111 (WP_135540314.1), Brevibacterium oceani (WP_210602990.1), Brevibacterium oceani (WP_181273084.1), Brevibacterium antiquum (WP_101620895.1), Brevibacterium sp. 239c (WP_101574167.1), Brevibacterium sp. FME17 (WP_193096434.1), Brevibacterium sp. FME37 (WP_193073001.1), Brevibacterium (WP_009376218.1), Brevibacterium permense (WP_173151746.1), Brevibacterium casei (WP_095376411.1), Brevibacterium casei (WP_119296730.1), Brevibacterium casei (WP_063249380.1), or Brevibacterium casei (WP_144588713.1).

In another example, E₂ may also be called lysine N(6)-hydroxylase/L-ornithine N(5)-oxygenase family protein or L-lysine 6-monooxygenase (dfoA) and may be from Actinospica acidiphila (NEA95037.1), Streptomyces sp. 15 (WP_199217164.1), Streptomyces sp. ZS0098 (WP_122217404.1), Streptomyces sp. RK31 (WP_210906078.1), Streptomyces sp. BSE7-9 (WP_128126623.1), Streptomyces sp. 14(2020) (WP_199206420.1), Streptomyces sp. PAM3C (WP_216716554.1), Streptomyces sp. E2N171 (WP_121720277.1), Streptomyces sp. E1N211 (WP_114873827.1), Streptomyces vinaceus (QL65622.1), Actinospica acidiphila (NEA78438.1), Streptomyces sp. Z38 (WP_156699446.1), Streptomyces sp. SMS_SU21 (WP_102641626.1), Streptomyces variabilis (WP_189366530.1), Streptomyces sp. HNS054 (WP_048457763.1), Streptomyces sp. 4F (ALV50328.1), Streptomyces matensis (GGT60880.1), Actinospica acidiphila (WP_203550773.1), Streptomyces cellulosae (GHE36673.1), Streptomyces griseorubens' (WP_037639705.1), Streptomyces griseorubens (GGQ93424.1), Streptomyces werraensis (WP_190000755.1), Streptomyces sp. di50b (SCD87686.1), Streptomyces sp. MNU103 (WP_230228098.1), Streptomyces werraensis (WP_225629336.1), Actinospica acidiphila (WP_163087763.1), Streptomyces sp. GESEQ-13 (WP_210633992.1), Streptomyces althioticus (GGQ52599.1), Streptomyces sp. XHT-2 (WP_161107407.1), Streptomyces (WP_028959209.1), Streptomyces albogriseolus (GHC05278.1), Streptomyces sp. McG8 (MBT2905834.1), Streptomyces (WP_086020175.1), Streptomyces sp. DH-12 (WP_106414114.1), Streptomyces (WP_093767215.1), Streptomyces sp. DH20 (WP_228916768.1), Streptomyces tendae (WP_150156576.1), Streptomyces sp. T12 (WP_145827643.1), Streptomyces (WP_215209974.1), Streptomyces (WP_006131547.1), Streptomyces sp. NRRL F-5527 (WP_031060218.1), Streptomyces pseudogriseolus (WP_225654116.1), Streptomyces pilosus (WP_189555971.1), Streptomyces malachitofuscus (WP_190164278.1), Streptomyces griseoloalbus (GGV56407.1), Streptomyces (WP_125506339.1), Streptomyces albaduncus (WP_184760471.1), Streptomyces (WP_161176537.1), Streptomyces calvus (WP_142232871.1), Streptomyces calvus (WP_142195206.1), Streptomyces viridosporus (WP_081236127.1), Streptomyces marokkonensis (WP_149548377.1), Streptomyces (WP_040907108.1), Streptomyces sp. NWU49 (WP_109540000.1), Streptomyces viridosporus (WP_004987747.1), Streptomyces fungicidicus (WP_121546080.1), Streptomyces sp. DH5 (WP_228966784.1), Streptomyces viridosporus (WP_016826156.1), Streptomyces sp. NA02536 (WP_176117311.1), Streptomyces sp. AC558_RSS880 (WP_217127653.1), Streptomyces sp. 13-12-16 (WP_085570641.1), Streptomyces sp. Tu 3180 (WP_159533060.1), Streptomyces sp. CB02400 (WP_073931738.1), Streptomyces griseoflavus (WP_190095098.1), Streptomyces capillispiralis (WP_145869103.1), Streptomyces (WP_184825089.1), Streptomyces sp. CHD11 (WP_215205176.1), Streptomyces sp. NRRL S-37 (WP_030869334.1), Streptomyces sp. SM1 (WP_103541522.1), Streptomyces toyocaensis (WP 037931691.1), Streptomyces poonensis (WP_189862605.1), Streptomyces sp. NRRL WC-3626 (WP_030217203.1), Streptomyces rubradiris (WP_189995711.1), Streptomyces (WP_086696892.1), Streptomyces corchorusii (WP_059261504.1), Streptomyces sp. EAS-AB2608 (WP_059249696.1), Streptomyces sp. FBKL.4005 (WP_094373911.1), Streptomyces hygroscopicus (WP_014672887.1), Streptomyces populi (WP_103552166.1), Streptomyces spongiae (WP_152774423.1), Streptomyces aurantiogriseus (WP_189941416.1), Streptomyces (WP_030825112.1), Streptomyces cyanogenus (WP 208032394.1), Streptomyces hygroscopicus (WP_058080637.1), Streptomyces griseoflavus Tu4000 (EFL41232.1), Streptomyces (WP_030782431.1), Streptomyces sp. Ru62 (WP_103810221.1), Streptomyces ferrugineus (WP_194045781.1), Streptomyces sp. M2CJ-2 (WP_202277307.1), Streptomyces hirsutus (WP_055628641.1), Streptomyces triticiradicis (WP_151470815.1), Streptomyces (WP_189754126.1), Streptomyces sp. NRRL WC-3725 (WP_031028778.1), Streptomyces phyllanthi (WP_152789148.1), Streptomyces hirsutus (WP_055595050.1), Streptomyces achromogenes (WP_030618669.1), Streptomyces sp. NRRL B-3648 (WP_053710495.1), Streptomyces (WP_031094998.1), Streptomyces sp. IMTB 2501 (WP_076088759.1), Pantoea (WP_010247061.1), Pantoea agglomerans (WP_158132335.1), Pantoea agglomerans (WP_163641339.1), Pantoea (WP_039389956.1), Pantoea agglomerans (WP_191924047.1), Pantoea agglomerans (WP_111534207.1), Pantoea agglomerans (WP_172608646.1), Pantoea agglomerans (WP_060679206.1), Pantoea agglomerans (WP_201500585.1), Pantoea agglomerans (WP_187500285.1), Pantoea agglomerans (WP_033787054.1), Pantoea (WP_124890595.1), Pantoea agglomerans (WP_163852070.1), Pantoea (WP_154210120.1), Pantoea agglomerans (WP_187506787.1), Pantoea agglomerans (WP_010670383.1), Pantoea agglomerans (WP_143789438.1), Pantoea sp. (RZK07466.1), Pantoea agglomerans (MBS7708199.1), Pantoea agglomerans (WP_089414761.1), Pantoea agglomerans (WP_182500641.1), Pantoea agglomerans (WP_193585707.1), Pantoea agglomerans (WP_191921679.1), Pantoea agglomerans (WP_163794175.1), Pantoea agglomerans (WP_208003523.1), Pantoea agglomerans (WP_064690801.1), Pantoea agglomerans (WP_069026760.1), Pantoea agglomerans (WP_033768947.1), Pantoea agglomerans (WP_086906736.1), Pantoea agglomerans (WP_062758876.1), Pantoea agglomerans (WP_191938763.1), Pantoea agglomerans (WP_163658683.1), Pantoea agglomerans (WP_163845107.1), Pantoea agglomerans (WP_163638697.1), Pantoea agglomerans (WP_115765005.1), Pantoea sp. WMus005 (WP_179898292.1), Pantoea (WP_039661441.1), Pantoea eucalypti (AWD37912.1), Pantoea (WP_105099754.1), Pantoea vagans (WP_095707818.1), Pantoea (WP_008924643.1), Pantoea vagans (WP_135910763.1), Pantoea sp. S62 (WP_201251339.1), Pantoea vagans (WP 107320322.1), Pantoea eucalypti (WP_187513086.1), Pantoea agglomerans (PE106058.1), Pantoea sp. PNT01 (WP_192377898.1), Pantoea eucalypti (AWD37907.1), Pantoea eucalypti (AWD37904.1), Pantoea jilinensis (QXG56954.1), Pantoea sp. OV426 (WP_090965310.1), Pantoea vagans (WP_161736341.1), Pantoea vagans (WP_061060758.1), Pantoea vagans (WP_033735770.1), Pantoea vagans (WP_013196309.1), Pantoea (WP_150011557.1), Pantoea agglomerans (WP_222925733.1), Pantoea (WP_046102688.1), Siphoviridae sp. (DAL43156.1), Type-F symbiont of Plautia stali (WP_058958199.1), Pantoea (WP_009092930.1), Pantoea sp. EKM101V (WP_167428608.1), Pantoea deleyi (WP 140917098.1), Pantoea sp. ARC607 (WP_111138385.1), Timema californicum (CAD7569022.1), Enterobacter soli (WP_014063925.1), Pantoea agglomerans (WP_233988771.1), Erwinia sp. 198 (WP_125287552.1), Erwinia sp. (HBV39119.1), Pantoea allii (WP_218994922.1), Pantoea sp. 3_1284 (WP_113655326.1), Pantoea sp. ICBG 1758 (WP_104085642.1), Pantoea (WP_063878329.1), Pantoea sp. PSNIH1 (WP_145339666.1), Pantoea sp. Ae16 (WP_071668352.1), Pantoea (WP_040113515.1), Pantoea sp. RIT 413 (WP_108569844.1), Pantoea eucrina (WP_065647647.1), Pantoea sp. Acro-807 (WP_166714195.1), Pantoea (WP_094111611.1), Pantoea allii (WP_096011383.1), Erwinia sp. 9145 (WP_034916568.1), Erwinia sp. ErVv1 (WP_067700592.1), Erwinia oleae (WP_034947936.1), Serratia sp. M24T3 (WP_009636530.1), Pantoea stewartia (WP_039338790.1), Rouxiella badensis (WP_165429960.1), Rouxiella badensis (WP_084912800.1), Pantoea (WP_110267872.1), Pantoea stewartia (WP_054634525.1), Rouxiella badensis (WP_227989822.1), Pantoea (WP_033740201.1), Pantoea stewartia (WP_058702088.1), Pantoea stewartia (WP_185199936.1), Erwinia tasmaniensis (WP_012442726.1), Erwinia piriflorinigrans (WP_023656444.1), Pantoea ananatis (WP_019106389.1), Pantoea ananatis (WP_03024531.1), Pantoea ananatis (WP_194761606.1), Pantoea ananatis (WP_064352874.1), Erwinia amylovora (WP_004160307.1), Erwinia amylovora (WP_099350830.1), Erwinia amylovora CFBP1430 (5O8P_A), Erwinia amylovora (WP_168394395.1), Erwinia amylovora (WP_168415207.1), Erwinia amylovora (WP_004171022.1), Erwinia pyrifoliae DSM 12163 (CAY75870.1), Erwinia pyrifoliae (WP_012669440.1), Erwinia sp. Ejp617 (WP_014542490.1), Erwinia typographi (WP_034899164.1), Erwinia (WP_013202517.1), Erwinia billingiae (WP_053142707.1), Pantoea ananatis (WP_210512032.1), Pantoea ananatis (WP_161611345.1), Pantoea ananatis (AWD37900.1), Pantoea (WP_014606631.1), Pantoea sp. BAV 3049 (WP_158784840.1), Pantoea ananatis (WP_029568444.1), Pantoea ananatis (WP_110956821.1), Pantoea ananatis (WP_105077110.1), Mixta mediterraneensis (WP_193405262.1), Pantoea ananatis (WP_210451676.1), Pantoea ananatis (WP_028724620.1), Erwinia psidii (WP_124232197.1), Pantoea ananatis (WP_058705541.1), Erwinia iniecta (WP_052902396.1), Pantoea sp. IMH (WP_024968324.1), Erwinia (WP_099707479.1), or Pantoea agglomerans (WP_111534207.1).

In particular, E₂ may be from Streptomyces coelicolor, Streptomyces violaceoruber, Streptomyces pilosus, Erwinia amylovora, Pantoea agglomerans, or Corynebacterium xerosis. More in particular, E₂ may be selected from the group consisting of Streptomyces coelicolor (NUV53723.1), Streptomyces violaceoruber, Streptomyces pilosus (WP_189555971.1), Erwinia amylovora (WP_004160307.1), Pantoea agglomerans (WP_010247061.1), or Corynebacterium xerosis (SLM95113.1). Even more in particular, E₂ may comprise at least 70% sequence identity relative to SEQ ID NO:4 (E₂ₐ), SEQ ID NO:16 (E_{2b}), SEQ ID NO:26 (E_{2c}), SEQ ID NO:33 (E_{2d}), SEQ ID NO:39 (E₂ₑ) or SEQ ID NO:45 (E_{2f}).

E₃ is a N5-Aminopentyl-N-(hydroxy)-succinamic acid synthase (EC: 2.3.-.-) capable of converting N5-hydroxy-cadaverine and succinyl-coenzyme A to N5-aminopentyl-N-(hydroxy)-succinamic acid. In particular, enzyme E₃ converts N5-hydroxy-cadaverine and succinyl-coenzyme A to N5-aminopentyl-N-(hydroxy)-succinamic acid. E₃ may be an acetyltransferase or a GNAT family N-acetyltransferase or also called a desferrioxamine E biosynthesis protein DesD of siderophore synthetase superfamily, group C, or siderophore synthetase component, ligase. More in particular, E₃ may be from Streptomyces (WP_048457762.1), Streptomyces sp. 14(2020) (WP_199206419.1), Streptomyces sp. 15 (WP_199217165.1), Streptomyces sp. Z38 (WP_156699445.1), Streptomyces sp. RK31 (WP_210906077.1), Actinospica acidiphila (WP_163087761.1), Streptomyces (WP_102641627.1), Streptomyces sp. di50b (SCD87678.1), Streptomyces werraensis (WP_190000756.1), Streptomyces griseorubens (GGQ93417.1), Streptomyces sp. MNU103 (WP_230228100.1), Streptomyces matensis (GGT60873.1), Streptomyces griseorubens (WP_033274713.1), Streptomyces (WP_136238514.1), Actinospica acidiphila (WP_203550772.1), Streptomyces (WP_215209975.1), Streptomyces cellulosae (GHE36667.1), Streptomyces (WP_019525183.1), Streptomyces tendae (WP_150156577.1), Streptomyces althioticus (GGQ52607.1), Streptomyces sp. GESEQ-13 (WP_210633991.1), Streptomyces sp. F-7 (WP_093767214.1), Streptomyces rubiginosus (WP_189480783.1), Streptomyces sp. T12 (WP_145827642.1), Streptomyces sp. DH20 (WP_228916770.1), Streptomyces viridodiastaticus (WP_189909874.1), Streptomyces (WP_006131548.1), Streptomyces sp. 2BBP-J2 (WP_167741843.1), Streptomyces pseudogriseolus (WP_225654117.1), Streptomyces sp. DH-12 (WP_106414113.1), Streptomyces pilosus (WP_189555972.1), Streptomyces marokkonensis (WP_149548376.1), Streptomyces sp. CB02400 (WP_073931737.1), Streptomyces (WP_164333385.1), Streptomyces sp. SM1 (WP_103541521.1), Streptomyces (WP_192229625.1), Streptomyces sp. CHD11 (WP_215205175.1), Streptomyces sp. AC558_RSS880 (WP_217127652.1), Streptomyces (WP_171113987.1), Streptomyces spinoverrucosus (WP_141313135.1), Streptomyces toyocaensis (WP_037931694.1), Streptomyces sp. 13-12-16 (WP_085570642.1), Streptomyces sp. DH5 (WP_228966783.1), Streptomyces griseoflavus (WP_004931114.1), Streptomyces sp. NA02536 (WP_176117312.1), Streptomyces (WP_142195205.1), Streptomyces spinoverrucosus (WP_196462535.1), Streptomyces sp. SLBN-134 (WP_142170642.1), Streptomyces lomondensis (WP_190048216.1), Streptomyces sp. JCM17656 (QWA22516.1), Streptomyces capillispiralis (WP_145869102.1), Streptomyces (WP_215154691.1), Streptomyces viridochromogenes (WP_004001040.1), Streptomyces (WP_125510363.1), Streptomyces chromofuscus (WP_189699031.1), Streptomyces griseoflavus (WP_190095097.1), Streptomyces (WP_126900012.1), Streptomyces sp. NRRL WC-3626 (WP_030217191.1), Streptomyces curacoi (WP_062155922.1), Streptomyces malachitofuscus (GGX09391.1), Streptomyces malachitofuscus (WP_190164476.1), Streptomyces afghaniensis 772 (EPJ38539.1), Streptomyces fungicidicus (WP_121546081.1), Streptomyces chartreusis (WP_107905561.1), Streptomyces sp. WAC 05379 (WP_125529404.1), Streptomyces wuyuanensis (WP_093654151.1), Streptomyces swartbergensis (WP_086603880.1), Streptomyces chartreusis (WP_176577002.1), Streptomyces bellus (WP_193505879.1), Streptomyces coeruleorubidus (WP_150481012.1), Streptomyces dysideae (WP_067021295.1), Streptomyces africanus (WP_086560822.1), Streptomyces iakyrus (WP_033308050.1), Streptomyces djakartensis (WP_190197820.1), Streptomyces sp. XY152 (WP_053636056.1), Streptomyces (WP_030825115.1), Streptomyces sp. SID5643 (WP_161369364.1), Streptomyces caelestis (WP_184984045.1), Streptomyces qaidamensis (WP_062927006.1), Streptomyces regalis (WP_062703120.1), Streptomyces umbrinus (WP_190227783.1), Streptomyces sp. NRRL S-146 (WP_031102640.1), Streptomyces umbrinus (WP_189843349.1), Streptomyces collinus (MBB5811277.1), Streptomyces sp. WAC04114 (WP_221757552.1), Streptomyces indiaensis (WP_234847835.1), Streptomyces massasporeus (WP_189588732.1), Streptomyces (WP_104779799.1), Streptomyces violaceochromogenes (WP_191845917.1), Streptomyces montanus (WP_138046192.1), Streptomyces sp. A244 (WP_107455124.1), Streptomyces (WP_031130500.1), Streptomyces paradoxus (WP_184558435.1), Streptomyces tuirus (WP_190899118.1), Streptomyces sp. NRRL S-475 (WP_030838074.1), Streptomyces hawaiiensis (WP_175432717.1), Streptomyces purpurascens (WP_189723836.1), Streptomyces janthinus (WP_193478232.1), Streptomyces sp. AK010 (WP_185013999.1), Streptomyces luteogriseus (WP_184910807.1), or Streptomyces pilosus (WP_189555972.1).

In one example, E₃ may be an acetyltransferase from Streptomyces sp. (QTK16920.1 GI:2021361637), Desulfosarcina cetonica (VTR67244.1 GI:2039686980), Streptomyces leeuwenhoekii (CQR63914.1 GI:822880124), Streptomyces sp. PAMC 26508 (AGJ55095.1 GI:478746515), Streptomyces scabiei 87.22 (CBG72817.1 GI:260649702), Streptomyces coelicolor A3(2) (CAB87221.1 GI:7544048), Streptomyces violaceorube, Pseudarthrobacter siccitolerans (CCQ47788.1 GI:635350230), Streptomyces ambofaciens (CAL18384.1 GI:115501978), Arthrobacter sp. 9AX (VXB93179.1 GI:17893988879, Streptomyces ambofaciens ATCC 23877 (AKZ55868.1 GI:917649199), Streptomyces formicae (ATL27941.1 GI:1259310060), Orrella dioscoreae (SOE50046.1 GI:1253556130), Nocardiopsis sp. JB363 (S1088002.1 GI:1143070537), Orrella dioscoreae (SBT27366.1 GI:1037119874), Streptomyces venezuelae (CUM41035.1 GI:932870024), Elizabethkingia anophelis NUHP1 (AIL46075.1 GI:675104782), Achromobacter xylosoxidans NBRC 15126=ATCC 27061 (AHC47452.1 GI:566051779), Desulfosarcina cetonica (VTR65358.1 GI:2039688767), Desulfosarcina cetonica (VTR65683.1 GI:2039688264), Desulfosarcina cetonica (VTR64064.1 GI:2039690162), Desulfosarcina cetonica (VTR64557.1 GI:2039689733), Desulfosarcina cetonica (VTR64494.1 GI:2039689672), Desulfosarcina cetonica (VTR65495.1 GI:2039688584), Desulfosarcina cetonica (VTR65880.1 GI:2039688461), Desulfosarcina cetonica (VTR65802.1 GI:2039688383), Desulfosarcina cetonica (VTR67337.1 GI:2039686714), Desulfosarcina cetonica (VTR67722.1 GI:2039686548), Desulfosarcina cetonica (VTR67458.1 GI:2039686284), Desulfosarcina cetonica (TR67453.1 GI:2039686279), Desulfosarcina cetonica (VTR68479.1 GI:2039685800), Desulfosarcina cetonica (VTR68453.1 GI:2039685774), Desulfosarcina cetonica (VTR69949.1 GI:2039683894), Desulfosarcina cetonica (VTR70394.1 GI:2039683642), Desulfosarcina cetonica (VTR70461.1 GI:2039683453), Desulfosarcina cetonica (VTR70443.1 GI:2039683435), Pimelobacter simplex (AIY15770.1 GI:723622294), Streptomyces sp. NL15-2K (CB49136.1 GI:1493708163), Aquitalea magnusonii (BBF86346.1 GI:1435241518), Micromonospora sp. B006 (AXO35036.1 GI:1450456452), Streptomyces venezuelae (ALO08593.1 GI:952470255), Streptomyces bottropensis ATCC 25435 (EMF50429.1 GI:456384851), Streptomyces venezuelae ATCC 10712 (CCA55858.1 GI:328882619), Drosophila melanogaster (CAA17683.1 GI:2924547), Desulfosarcina cetonica (VTR64151.1 GI:2039689961), Desulfosarcina cetonica (VTR64706.1 GI:2039689502), Desulfosarcina cetonica (VTR65910.1 GI:2039688142), Desulfosarcina cetonica (VTR65903.1 GI:2039688135), Desulfosarcina cetonica (VTR68069.1 GI:2039685916), Desulfosarcina cetonica (VTR69458.1 GI:2039684725), Desulfosarcina cetonica (VTR63766.1 GI:2039690355), Desulfosarcina cetonica (VTR64871.1 GI:2039689205), Desulfosarcina cetonica (VTR64990.1 GI:2039688983), Desulfosarcina cetonica (VTR65346.1 GI:2039688755), Desulfosarcina cetonica (VTR65983.1 GI:2039688006), Desulfosarcina cetonica (VTR66132.1 GI:2039687832), Desulfosarcina cetonica (VTR67865.1 GI:2039686223), Desulfosarcina cetonica (VTR67945.1 GI:2039686120), Desulfosarcina cetonica (VTR68557.1 GI:2039685499), Desulfosarcina cetonica (VTR69047.1 GI:2039685372), Desulfosarcina cetonica (VTR68952.1 GI:2039685277), Desulfosarcina cetonica (VTR69234.1 GI:2039684917), Desulfosarcina cetonica (VTR69164.1 GI:2039684847), Desulfosarcina cetonica (VTR69124.1 GI:2039684807), Desulfosarcina cetonica (VTR69451.1 GI:2039684718), Desulfosarcina cetonica (VTR69548.1 GI:2039684504), Desulfosarcina cetonica (VTR70126.1 GI:2039684068), Desulfosarcina cetonica (VTR70315.1 GI:2039683696), Desulfosarcina cetonica (VTR70566.1 GI:2039683558), Streptomyces malaysiensis (ATL84885.1 GI:1266921847), Streptomyces fulvissimus DSM 40593 (AGK77333.1 GI:485094150), Brevibacterium oceani (WP_181273085.1), Brevibacterium sp. HY170 (WP_231442296.1), Brevibacterium sp. FME17 (WP_193096435.1), Brevibacterium casei (WP_009376215.1), Brevibacterium pigmentatum (WP_167198399.1), Brevibacterium atlanticum (WP_166973259.1), Brevibacterium (WP_137827008.1), Brevibacterium sp. S22 (TGD29889.1), Brevibacterium epidermidis (WP_098731557.1), Brevibacterium linens (HJF75768.1), Brevibacterium sp. LS14 (WP_131248027.1), Brevibacterium sp. VCM10 (WP_025776856.1), Brevibacterium sp. UCMA 11752 (WP_235350998.1), Brevibacterium permense (WP_173151748.1), Brevibacterium aurantiacum (WP_125240720.1), Brevibacterium aurantiacum (WP_193078849.1), Brevibacterium aurantiacum (WP_096145960.1), Brevibacterium aurantiacum (WP_114384949.1), Brevibacterium siliguriense (WP_092011616.1), Brevibacterium aurantiacum (WP_096160424.1), Brevibacterium aurantiacum (WP_193086563.1), Brevibacterium sp. ‘Marine’ (WP_169252963.1), Brevibacterium aurantiacum (WP_135446718.1), Brevibacterium aurantiacum (WP_193084516.1), Brevibacterium marinum (WP_167950434.1), Brevibacterium aurantiacum (WP_101639105.1), Brevibacterium limosum (WP_166822893.1), Brevibacterium aurantiacum (WP_096167624.1), Brevibacterium aurantiacum (WP_143924263.1), Brevibacterium aurantiacum (WP_096178868.1), Brevibacterium aurantiacum (WP_096157397.1), Brevibacterium aurantiacum (WP_096163052.1), Brevibacterium aurantiacum (WP_009883550.1), Brevibacterium aurantiacum (WP_069600709.1), Brevibacterium aurantiacum (WP_101557621.1), Brevibacterium casei (WP_119296729.1), Brevibacterium sp. CFH 10365 (WP_152347364.1), Brevibacterium aurantiacum (WP_101598306.1), Brevibacterium casei (WP_082834881.1), Brevibacterium casei (WP_144588711.1), Agreia bicolorata (WP_078715081.1), Brevibacterium sp. SMBL_HHYL_HB1 (WP_212129586.1), Brevibacterium casei (KZE22294.1), Brevibacterium casei (WP_232623671.1), Brevibacterium casei (QQB16215.1), Agreia bicolorata (WP_044440798.1), Mycobacteroides abscessus subsp. Abscessus (SIH96297.1), Brevibacterium casei (WP_101624625.1), Brevibacterium casei (WP_190247010.1), Brevibacterium sp. S111 (WP_135540312.1), Brevibacterium sp. W7.2 (WP_211979250.1), Corynebacterium xerosis (SLM95104.1 GI:1188028261), or from a plant metagenome with accession number GO10018.1 GI:1613564736, VFR50243.1 GI:1591769475, VFR49444.1 GI:1591759418, VFR42273.1 GI:1591739264, VFR87867.1 GI:1591734832, VFR79172.1 GI:1591731713, VFR90674.1 GI:1591727121, VFR73697.1 GI:1591713709, VFR35670.1 GI:1591707861, VFR17966.1 GI:1591706236, VFR24793.1 GI:1591690017, or VFR74915.1 GI:1591683123.

In another example, E₃ may be desferrioxamine siderophore biosynthesis protein dfoC from Pantoea agglomerans (AWD37890.1), Pantoea agglomerans (WP_187507962.1), Pantoea agglomerans (HAT00136.1), Pantoea agglomerans (WP_010247060.1), Pantoea agglomerans (WP_201500586.1), Pantoea agglomerans (WP_124890596.1), Pantoea agglomerans (WP_187496373.1), Pantoea agglomerans (WP_163852072.1), Pantoea agglomerans (WP_154210121.1), Pantoea agglomerans (WP_033760513.1), Pantoea agglomerans (WP_187512465.1), Pantoea agglomerans (WP_207253898.1), Pantoea agglomerans (WP_187495053.1), Pantoea agglomerans i(WP_060679208.1), Pantoea sp. EKM10T (WP_167430078.1), Pantoea agglomerans (WP_187500286.1), Pantoea agglomerans (WP_187492242.1), Pantoea agglomerans (WP_163763414.1), Pantoea agglomerans (WP_122459787.1), Pantoea agglomerans (WP_061902164.1), Pantoea vagans (WP_061703165.1), Pantoea agglomerans (MBS7708200.1), Pantoea (WP_159226815.1), Pantoea agglomerans (WP_187513802.1), Pantoea vagans (WP_050679331.1), Pantoea agglomerans (WP_187484792.1), Pantoea agglomerans (WP_163799890.1), Pantoea sp. OVA07A (WP_223565934.1), Pantoea agglomerans (WP_191917404.1), Pantoea agglomerans (WP_033773381.1), Pantoea agglomerans (WP_064690802.1), Pantoea agglomerans (WP_111534208.1), Pantoea agglomerans (WP_223564415.1), Pantoea agglomerans (WP_129063349.1), Pantoea agglomerans (WP_191920297.1), Pantoea agglomerans (WP_163641337.1), Pantoea agglomerans (WP_022624353.1), Pantoea agglomerans (WP_187492906.1), Pantoea agglomerans (WP_157353121.1), Pantoea sp. (RZK07467.1), Pantoea agglomerans (WP_191914122.1), Pantoea agglomerans (WP_045140091.1), Pantoea sp. EKM20T (WP_167433056.1), Pantoea agglomerans (WP_187501430.1), Pantoea (WP_167423161.1), Pantoea agglomerans (WP_191921680.1), Pantoea agglomerans (WP_163658684.1), Pantoea agglomerans (WP_172608645.1), Pantoea agglomerans (WP_192073070.1), Pantoea sp. CFSAN033090 (WP_052270704.1), Pantoea agglomerans (WP_187511066.1), Pantoea agglomerans (WP_158149880.1), Pantoea agglomerans (WP_132499089.1), Pantoea agglomerans (MBT8497800.1), Curtobacterium plantarum (WP_122889392.1), Pantoea agglomerans (WP_192033741.1), Pantoea agglomerans (WP_033768945.1), Pantoea agglomerans (WP_187515506.1), Pantoea agglomerans (WP_182505410.1), Pantoea agglomerans (WP_182500640.1), Pantoea agglomerans (WP_158132336.1), Pantoea agglomerans (WP_010670384.1), Pantoea agglomerans (WP_187506786.1), Pantoea agglomerans (WP_179256483.1), Pantoea agglomerans (WP_115765004.1), Pantoea agglomerans (WP_191922816.1), Pantoea agglomerans (WP_191914536.1), Pantoea agglomerans (AWD37885.1), Pantoea agglomerans (WP_033780267.1), Pantoea agglomerans (WP_062758877.1), Pantoea agglomerans (WP_115388310.1), Pantoea agglomerans (WP_089414760.1), Pantoea agglomerans (WP_191918109.1), Pantoea agglomerans (WP_137228077.1), Pantoea agglomerans (WP_039389954.1), Pantoea agglomerans (WP_191924048.1), Pantoea agglomerans (WP_191920726.1), Pantoea agglomerans (WP_163845109.1), Pantoea sp. paga (WP_143990819.1), Pantoea agglomerans (WP_031590726.1), Pantoea agglomerans (WP_143789436.1), Pantoea vagans (WP_083069696.1), Pantoea agglomerans (WP 033787056.1), Pantoea agglomerans (WP 208003520.1), Pantoea agglomerans (WP_235765353.1), Pantoea pleuroti (WP_182686862.1), Pantoea agglomerans (WP_069026761.1), Pantoea agglomerans (WP_163638696.1), Pantoea agglomerans (WP_208442935.1), Pantoea agglomerans (WP_207091826.1), Pantoea agglomerans (WP_064702866.1), Pantoea agglomerans (WP_086906737.1), Pantoea agglomerans (KEY40395.1), Pantoea sp. OV426 (WP_090965308.1), Pantoea (WP_150037408.1), Pantoea eucalypti (AWD37908.1), Pantoea (WP_150011559.1), Pantoea sp. WMus005 (WP_179898291.1), Pantoea eucalypti (AWD37911.1), Pantoea vagans (WP_161736340.1), Erwinia amylovora (WP_004160308.1), Erwinia amylovora (QJQ67970.1 GI:1839296356), Erwinia amylovora (QJQ64271.1 GI:1839292579), Erwinia amylovora (QJQ60469.1 GI:1839288766), Erwinia amylovora (QJQ56770.1 GI:1839285065), Erwinia amylovora (QJQ53072.1 GI:1839281365), Xenorhabdus cabanillasii JM26 (PHM78217.1 GI:1269095937), Xenorhabdus cabanillasii JM26 (PHM78208.1 GI:1269095928), Xenorhabdus hominickii (PHM56127.1 GI:1269072847), Xenorhabdus mauleonii (PHM37952.1 GI:1269054189), Xenorhabdus mauleonii (PHM37944.1 GI:1269054181), Xenorhabdus budapestensis (PHM29821.1 GI:1269045843), Xenorhabdus budapestensis (PHM29812.1 GI:1269045834), Erwinia amylovora CFBP1430 (CBA23308.1 GI:291555140), Erwinia pyrifoliae DSM 12163 (CAY75871.1 GI:283479955), Erwinia amylovora LA637 (CDK23310.1 GI:566688504), Erwinia amylovora LA636 (CDK19939.1 GI:565455063), Erwinia amylovora LA635 (CDK16572.1 GI:565423714), Erwinia piriflorinigrans CFBP 5888 (CCG88689.1 GI:560105768), Erwinia amylovora MR1 (CCP08595.1 GI:478726734), Erwinia amylovora Ea644 (CCP04532.1 GI:478723237), Erwinia amylovora UPN527 (CCP00585.1 GI:478719346), Erwinia amylovora NBRC 12687=CFBP 1232 (00095265.1 GI:478715307), Erwinia amylovora 01SFR-BO (00091470.1 GI:478711918), Erwinia amylovora CFBP 2585 (00087679.1 GI:478708144), Erwinia amylovora Ea266 (00083917.1 GI:478703932), Erwinia amylovora Ea356 (00080113.1 GI:478700098), Erwinia amylovora ACW56400 (EKV52685.1 GI:426274945), or Erwinia amylovora ATCC BAA-2158 (CBX82136.1 GI:312173882).

In particular, E₃ is selected from the group consisting of DesC from Streptomyces coelicolor, DesC from Streptomyces pilosus, S. violaceorube, N-terminal domains of the DesC proteins from Corynebacterium xerosis, Pantoea agglomerans, or Erwinia amylovora. More in particular, the E₃ is selected from the group consisting of Streptomyces coelicolor, Streptomyces violaceorube, Streptomyces pilosus (WP_189555972.1), Corynebacterium xerosis (SLM95104.1 GI:1188028261), Pantoea agglomerans (AWD37890.1) and Erwinia amylovora (WP_004160308.1). Even more in particular, E₃ comprises at least 70% sequence identity relative to SEQ ID NO:5 (E₃ₐ), SEQ ID NO:17 (E_{3b}), SEQ ID NO:27 (E_{3c}), N-terminal domain of SEQ ID N046 (E_{3d}), or SEQ ID N0:40 (E₃ₑ) or SEQ ID NO:34 (E_{3f}).

The cell according to any aspect of the present invention, is further genetically modified to or comprises a further genetic modification that increases expression relative to a wild-type cell of at least one E₄ enzyme selected from the group consisting of:
desferrioxamine synthetase (EC 6.3.-.-) (E₄ᵢ) capable of converting N5-aminopentyl-N-(hydroxyl)-succinamic acid to desferrioxamine B or H;
desferrioxamine synthetase (EC 6.3.-.-) (E₄ᵢᵢ) capable of converting N5-aminopentyl-N-(hydroxyl)-succinamic acid to at least one desferrioxamine;
bisucaberin synthetase (EC 6.3.-.-) (E₄ᵢᵢᵢ) capable of converting N5-aminopentyl-N-(hydroxyl)-succinamic acid to bisucaberin B; and
bisucaberin synthetase (EC 6.3.-.-) (E₄ᵢᵥ) capable of converting N5-aminopentyl-N-(hydroxyl)-succinamic acid to bisucaberin.

In particular, E₄ᵢ, E₄ᵢᵢ, E₄ᵢᵢᵢ or E₄ᵢᵥ, may also be called desferrioxamine E biosynthesis protein (DesD) which is usually from the siderophore synthetase superfamily, group C, siderophore synthetase component, ligase. E₄ᵢ, E₄ᵢᵢ, E₄ᵢᵢᵢ or E₄ᵢᵥ may also be called lucA/lucC family siderophore biosynthesis protein. In particular, E₄ᵢ, E₄ᵢᵢ, E₄ᵢᵢᵢ or E₄ᵢᵥ, may be DesD from Streptomyces coelicolor (NUV53721.1), Streptomyces sp. 15 (WP_199217166.1), Streptomyces sp. PAM3C (WP_216716553.1), Streptomyces sp. ZS0098 (WP_122217405.1), Streptomyces vinaceus (MQL65620.1), Streptomyces sp. RK31 (WP_210906076.1), Streptomyces sp. E₁N211 (WP_114873828.1), Streptomyces variabilis (WP_189366531.1), Streptomyces sp. HNS054 (WP_048457761.1), Actinospica acidiphil (NEA78436.1), Streptomyces sp. E2N171 (WP_121720278.1), Streptomyces sp. Z38 (WP_156699444.1), Streptomyces sp. 14(2020) (WP_199206418.1), Streptomyces sp. BSE7-9 (WP_199215200.1), Streptomyces sp. SMS_SU21 (WP_102641628.1), Streptomyces sp. MNU103 (WP_230228103.1), Streptomyces matensis (GGT60866.1), Streptomyces griseorubens (GGQ93410.1), Streptomyces althioticus (GGQ52613.1), Actinospica acidiphila (WP_203550771.1), Streptomyces sp. 4F (ALV50330.1), Actinospica acidiphila (WP_163087759.1), Streptomyces griseorubens (WP_037639707.1), Streptomyces sp. di50b (SCD87662.1), Streptomyces (WP_167741844.1), Streptomyces sp. F-7 (WP_093767213.1), Streptomyces albogriseolus (GHC05289.1), Streptomyces cellulosae (GHE36661.1), Streptomyces werraensis (GHE82670.1), Streptomyces sp. DH20 (WP_228916771.1), Streptomyces werraensis (WP_225641487.1), Streptomyces werraensis (WP_225629337.1), Streptomyces tendae (WP_150156578.1), Streptomyces pseudogriseolus (WP_225654118.1), Streptomyces sp. NRRL F-5527 (WP_031060221.1), Streptomyces sp. T12 (WP_145827641.1), Streptomyces sp. GESEQ-13 (WP_210633990.1), Streptomyces gancidicus (WP_006131549.1), Streptomyces sp. NRRL S-1314 (WP_031017402.1), Streptomyces (WP_215209976.1), Streptomyces pseudogriseolus group 8WP_189406281.19, Streptomyces (WP_028959208.1), Streptomyces sp. S-9 (WP_203350072.1), Streptomyces sp. McG8 (WP_215188574.1), Streptomyces (WP_019525182.1), Streptomyces sp. DH-12 (WP_106414112.1), Streptomyces marokkonensis (WP_149548375.1), Streptomyces malachitofuscus (WP_190164277.1), Streptomyces (WP_142195204.1), Streptomyces calvus (WP_142232870.1), Streptomyces sp. SM1 (WP_103541520.1), Streptomyces toyocaensis (WP_037931697.1), Streptomyces sp. CHD11 (WP_215205174.1), Streptomyces (WP_030825118.1), Streptomyces sp. NRRL S-37 (WP_030869328.1), Streptomyces sp. AC558_RSS880 (WP_217127651.1), Streptomyces sp. DH5 (WP_228966782.1), Streptomyces pilosus (WP_189555973.1), Streptomyces pilosus (WP_189595561.1), Streptomyces griseoflavus (WP_190095096.1), Streptomyces sp. NRRL WC-3626 (WP_030217188.1), Streptomyces sp. 13-12-16 (WP_085570643.1), Streptomyces capillispiralis (WP_145869101.1), Streptomyces griseoflavus (WP_004931111.1), Streptomyces sp. CB02400 (WP_073931736.1), Streptomyces sp. SLBN-134 (WP_142170640.1), Streptomyces (WP_125506338.1), Streptomyces sp. NA02536 (WP_176117313.1), Streptomyces fungicidicus (WP_121546082.1), Streptomyces leeuwenhoekii (WP_048573206.1), Streptomyces leeuwenhoekii (WP_029382123.1), Streptomyces albaduncus (WP_184760469.1), Streptomyces viridochromogenes (WP_048584025.1), Streptomyces viridochromogenes (WP_053200122.1), Streptomyces (WP_215154693.1), Streptomyces viridochromogenes (WP_004001039.1), Streptomyces sp. ISL-14 (MBT2673677.1), Streptomyces cyaneogriseus (WP_044381715.1), Streptomyces griseomycini (WP_193473351.1), Streptomyces curacoi (WP_062155921.1), Streptomyces sp. uw30 (WP_147999450.1), Streptomyces caeruleatus (WP_062720821.1), Streptomyces harenosi (WP_164393243.1), Streptomyces griseostramineus (WP_184825094.1), Streptomyces bicolor (WP_031478546.1), Streptomyces regali (WP_062703122.1), Streptomyces sp. cf386 (WP_093908265.1), Streptomyces azureus (WP_059420174.1), Streptomyces chartreusis (WP_150502416.1), Streptomyces swartbergensis (WP_086603881.1), Streptomyces sp. CB02414 (WP_073723308.1), Streptomyces chartreusis (WP_176577001.1), Streptomyces sp. KS_5 (WP_030944363.1), Streptomyces sp. Tu102 (WP_214335617.1), Streptomyces sp. (NUT26959.1), Streptomyces cyaneochromogenes (WP_126392412.1), Streptomyces spinoverrucosus (WP_196462534.1), Streptomyces sp. HGB0020 (WP_016434996.1), Streptomyces coeruleorubidus (WP_150481013.1), Streptomyces sp. TRM S81-3 (WP_188181662.1), Streptomyces formicae (ATL27942.1 GI:1259310061), Micrococcus luteus Mu201 (SJN33943.1 GI:1145375606), Tenacibaculum mesophilum (BAX07670.1 GI:1172208181), Tenacibaculum mesophilum (BAX07668.1 GI:1172208179), Gammaproteobacteria bacterium (BAL45578.1 GI:371940279), Nocardiopsis sp. JB363 (S1088003.1 GI:1143070538), Enterobacteriaceae bacterium bta3-1 (AJR02043.1 GI:764043809), Streptomyces venezuelae (CUM41034.1 GI:932870023), Elizabethkingia anophelis NUHP1 (AIL46076.1 GI:675104783), Streptomyces sp. PAMC 26508 (AGJ55096.1 GI:478746516), Halomonas sp. TD01 (CAH1042919.1 GI:2174847834), Corynebacterium xerosis (SLM95104.1 GI:1188028261), Streptomyces sp. CS113 (WP_087809199.1 GI:1205101816), Streptomyces anthocyanicus (WP_189283040.1 GI:1906517697), Streptomyces (WP_161377959.1 GI:1801827430), Streptomyces (WP_121702379.1 GI:1495472518), Streptomyces sp. LBUM 1480 (QTU54121.1 GI:2025348499), Streptomyces griseofuscus (QNT95273.1 GI:1906918764), Streptomyces (WP_106517219.1 GI:1370674867), Streptomyces anthocyanicus (WP_191849566.1 GI:1912958734), Streptomyces tendae (WP_189742138.1 GI:1907050264), Streptomyces (WP_164410815.1 GI:1817780094), Streptomyces lincolnensis (QMV07193.1 GI:1886115140), Streptomyces coelicolor (QKN66532.1 GI:1851833442), Streptomyces tendae (WP_164458103.1 GI:1817832929), Streptomyces rubrogriseus (WP_164277235.1 GI:1817618112), Streptomyces sp. SID10362 (WP_165285357.1 GI:1819611045), Streptomyces (WP_030400073.1 GI:663404431), Streptomyces (WP_011028585.1 GI:499338877), Streptomyces tendae (WP_159327807.1 GI:1797887667), Streptomyces coelicolor A3(2) (QFI42889.1 GI:1759037058), Streptomyces (WP_093456207.1 GI:1225447207), Streptomyces sp. LRa12 (WP_136207792.1 GI:1621656934), Streptomyces sp. E5N91 (WP_121709362.1 GI:1495480291), Streptomyces rubrogriseus (WP_109032792.1 GI:1389010967), Streptomyces sp. MH60 (WP_104632151.1 GI:1351098821), Streptomyces diastaticus (WP_102929444.1 GI:1332972289), Streptomyces sp. M1013 (WP_076974458.1 GI:1141434617), Streptomyces canus (WP_059300524.1 GI:976140927), Streptomyces violaceoruber (WP_030866491.1 GI:664338305), Streptomyces (WP 003976015.1 GI:490073840), Streptomyces ipomoeae (TQE33528.1 GI:1697190356), Streptomyces ipomoeae (TQE25637.1 GI:1697182207), Streptomyces sporangiiformans (TPQ23018.1 GI:1693045467), Streptomyces sp. Akac8 (THC55740.1 GI:1616616223), Streptomyces sp. LRa12 (THA97810.1 GI:1616076702), Streptomyces sp. H23 (WP_134652861.1 GI:1604860203), Streptomyces griseoviridis (AZS87080.1 GI:1547213271), Achromobacter xylosoxidans NBRC 15126=ATCC 27061 (AHC47453.1 GI:566051780), Streptomyces lividans 1326 (EOY47845.1 GI:509518532), Streptomyces collinus (UJA16738.1 GI:2179595515), Streptomyces collinus (UJA08397.1 GI:2179587162), Streptomyces hyderabadensis (WP_226024665.1 GI:2112773893), Streptomyces sp. S10(2018) (WP_127893570.1 GI:1553510828), Streptomyces sp. NL15-2K (GCB49135.1 GI:1493708162), Streptomyces scabiei 87.22 (CBG72816.1 GI:260649701), Micromonospora sp. B006 (AX035037.1 GI:1450456453), Streptomyces venezuelae (ALO08594.1 GI:952470256), Brachybacterium sp. SW0106-09 (GAP78407.1 GI:926973312), Pseudoalteromonas sp. SW0106-04 (GAP73945.1 GI:924441201), Elizabethkingia anopheles (KMU63886.1 GI:874590592), or Streptomyces venezuelae ATCC 10712 (CCA55859.1 GI:328882620).

In another example, E₄ᵢ, E₄ᵢᵢ, E₄ᵢᵢᵢ or E₄ᵢᵥ may also be called Chain A or B, desferrioxamine E biosynthesis protein DesD. In this example, the DesD may be the sequence with accession number 6XRC_B GI:1950842083 or 6XRC_A GI:1950842082 or 6NL2_B GI:179970866 or 6NL2_A GI:1799708661. The DesD may also be from Actinokineospora spheciospongiae (EWC61612.1 GI:583002104), Pimelobacter simplex (IY15768.1 GI:723622292), Aquitalea magnusonii (BBF86345.1 GI:1435241517), or Salinisphaera sp. LB1 (AWN17872.1 GI:1393627878).

In yet another example, E₄ᵢ, E₄ᵢᵢ, E₄ᵢᵢᵢ or E₄ᵢᵥ may also be called desferrioxamine E biosynthesis protein DesC at siderophore synthetase small component, acetyltransferase. The DesC may be from a plant metagenome with accession number VGO10018.1 GI:1613564736, VFR50243.1 GI:1591769475, VFR49444.1 GI:1591759418, VFR42273.1 GI:1591739264, VFR87867.1 GI:1591734832, VFR79172.1 GI:1591731713, VFR90674.1 GI:1591727121, VFR73697.1 GI:1591713709, VFR35670.1 GI:1591707861, VFR17966.1 GI:1591706236, VFR24793.1 GI:1591690017, or VFR74915.1 GI:1591683123, or from Streptomyces formicae (ATL27941.1 GI:1259310060), Orrella dioscoreae (SOE50046.1 GI:1253556130), Nocardiopsis sp. JB363 (S1088002.1 GI:1143070537), Orrella dioscoreae (SBT27366.1 GI:1037119874), Streptomyces venezuelae (CUM41035.1 GI:932870024), Elizabethkingia anophelis NUHP1 (AIL46075.1 GI:675104782), Achromobacter xylosoxidans NBRC 15126=ATCC 27061 (AHC47452.1 GI:566051779), Streptomyces lividans 1326 (EOY47844.1 GI:509518531), Streptomyces sp. PAMC 26508 (AGJ55095.1 GI:478746515), Pimelobacter simplex (AIY15770.1 GI:723622294), Streptomyces sp. NL15-2K (GCB49136.1 GI:1493708163), or Aquitalea magnusonii (BBF86346.1 GI:1435241518).

In a further example, E₄ᵢ, E₄ᵢᵢ, E₄ᵢᵢᵢ or E₄ᵢᵥ may also be called Chain D, desferrioxamine siderophore biosynthesis protein dfoC with accession number 5070_D GI:1351638365 or Chain C, desferrioxamine siderophore biosynthesis protein dfoC with accession number 5070_C GI:1351638364 or Chain B, desferrioxamine siderophore biosynthesis protein dfoC with accession number 5070_B GI:1351638363 or Chain A, desferrioxamine siderophore biosynthesis protein dfoC with accession number 5070_A GI:1351638362 or desferrioxamine siderophore biosynthesis protein dfoC from Pantoea agglomerans (AWD37890.1), Pantoea agglomerans (WP_187507962.1), Pantoea agglomerans (HAT00136.1), Pantoea agglomerans (WP_010247060.1), Pantoea agglomerans (WP_201500586.1), Pantoea agglomerans (WP_124890596.1), Pantoea agglomerans (WP_187496373.1), Pantoea agglomerans (WP_163852072.1), Pantoea agglomerans (WP_154210121.1), Pantoea agglomerans (WP_033760513.1), Pantoea agglomerans (WP_187512465.1), Pantoea agglomerans (WP_207253898.1), Pantoea agglomerans (WP_187495053.1), Pantoea agglomerans i(WP_060679208.1), Pantoea sp. EKM10T (WP_167430078.1), Pantoea agglomerans (WP_187500286.1), Pantoea agglomerans (WP_187492242.1), Pantoea agglomerans (WP_163763414.1), Pantoea agglomerans (WP_122459787.1), Pantoea agglomerans (WP_061902164.1), Pantoea vagans (WP_061703165.1), Pantoea agglomerans (MBS7708200.1), Pantoea (WP_159226815.1), Pantoea agglomerans (WP_187513802.1), Pantoea vagans (WP_050679331.1), Pantoea agglomerans (WP_187484792.1), Pantoea agglomerans (WP_163799890.1), Pantoea sp. OVA07A (WP_223565934.1), Pantoea agglomerans (WP_191917404.1), Pantoea agglomerans (WP_033773381.1), Pantoea agglomerans (WP_064690802.1), Pantoea agglomerans (WP_111534208.1), Pantoea agglomerans (WP_223564415.1), Pantoea agglomerans (WP_129063349.1), Pantoea agglomerans (WP_191920297.1), Pantoea agglomerans (WP_163641337.1), Pantoea agglomerans (WP_022624353.1), Pantoea agglomerans (WP_187492906.1), Pantoea agglomerans (WP_157353121.1), Pantoea sp. (RZK07467.1), Pantoea agglomerans (WP_191914122.1), Pantoea agglomerans (WP_045140091.1), Pantoea sp. EKM20T (WP_167433056.1), Pantoea agglomerans (WP_187501430.1), Pantoea (WP_167423161.1), Pantoea agglomerans (WP_191921680.1), Pantoea agglomerans (WP_163658684.1), Pantoea agglomerans (WP_172608645.1), Pantoea agglomerans (WP_192073070.1), Pantoea sp. CFSAN033090 (WP_052270704.1), Pantoea agglomerans (WP_187511066.1), Pantoea agglomerans (WP_158149880.1), Pantoea agglomerans (WP_132499089.1), Pantoea agglomerans (MBT8497800.1), Curtobacterium plantarum (WP_122889392.1), Pantoea agglomerans (WP_192033741.1), Pantoea agglomerans (WP_033768945.1), Pantoea agglomerans (WP_187515506.1), Pantoea agglomerans (WP_182505410.1), Pantoea agglomerans (WP_182500640.1), Pantoea agglomerans (WP_158132336.1), Pantoea agglomerans (WP_010670384.1), Pantoea agglomerans (WP_187506786.1), Pantoea agglomerans (WP_179256483.1), Pantoea agglomerans (WP_115765004.1), Pantoea agglomerans (WP_191922816.1), Pantoea agglomerans (WP_191914536.1), Pantoea agglomerans (AWD37885.1), Pantoea agglomerans (WP_033780267.1), Pantoea agglomerans (WP_062758877.1), Pantoea agglomerans (WP_115388310.1), Pantoea agglomerans (WP_089414760.1), Pantoea agglomerans (WP_191918109.1), Pantoea agglomerans (WP_137228077.1), Pantoea agglomerans (WP_039389954.1), Pantoea agglomerans (WP_191924048.1), Pantoea agglomerans (WP_191920726.1), Pantoea agglomerans (WP_163845109.1), Pantoea sp. paga (WP_143990819.1), Pantoea agglomerans (WP_031590726.1), Pantoea agglomerans (WP_143789436.1), Pantoea vagans (WP_083069696.1), Pantoea agglomerans (WP_033787056.1), Pantoea agglomerans (WP 208003520.1), Pantoea agglomerans (WP 235765353.1), Pantoea pleuroti (WP_182686862.1), Pantoea agglomerans (WP_069026761.1), Pantoea agglomerans (WP_163638696.1), Pantoea agglomerans (WP_208442935.1), Pantoea agglomerans (WP_207091826.1), Pantoea agglomerans (WP_064702866.1), Pantoea agglomerans (WP_086906737.1), Pantoea agglomerans (KEY40395.1), Pantoea sp. OV426 (WP_090965308.1), Pantoea (WP_150037408.1), Pantoea eucalypti (AWD37908.1), Pantoea (WP_150011559.1), Pantoea sp. WMus005 (WP_179898291.1), Pantoea eucalypti (AWD37911.1), Pantoea vagans (WP_161736340.1), Erwinia amylovora (QJQ67970.1 GI:1839296356), Erwinia amylovora (QJQ64271.1 GI:1839292579), Erwinia amylovora (QJQ60469.1 GI:1839288766), Erwinia amylovora (QJQ56770.1 GI:1839285065), Erwinia amylovora (QJQ53072.1 GI:1839281365), Erwinia amylovora (WP_004160308.1), Erwinia amylovora CFBP1430 (5O7O_A), Erwinia amylovora (QJQ53072.1), Erwinia amylovora NBRC 12687=CFBP 1232 (GAJ89919.1), Erwinia amylovora (WP_168428660.1), Erwinia amylovora (WP_099350829.1), Erwinia amylovora (WP_168384929.1), Erwinia amylovora (WP_168404295.1), Erwinia amylovora MR1 (CCP08595.1), Erwinia amylovora (WP_004171023.1), Erwinia amylovora (WP_168402725.1), Erwinia amylovora (WP_168394396.1), Erwinia sp. Ejp617 (ADP10409.1), Erwinia sp. Ejp617 (WP_041474304.1), Erwinia pyrifoliae (WP_012669441.1), Erwinia piriflorinigrans (WP_023656445.1), Erwinia tasmaniensis (WP_012442727.1), Xenorhabdus cabanillasii JM26 (PHM78217.1 GI:1269095937), Xenorhabdus cabanillasii JM26 (PHM78208.1 GI:1269095928), Xenorhabdus hominickii (PHM56127.1 GI:1269072847), Xenorhabdus mauleonii (PHM37952.1 GI:1269054189), Xenorhabdus mauleonii (PHM37944.1 GI:1269054181), Xenorhabdus budapestensis (PHM29821.1 GI:1269045843), Xenorhabdus budapestensis (PHM29812.1 GI:1269045834), Erwinia amylovora CFBP1430 (CBA23308.1 GI:291555140), Erwinia pyrifoliae DSM 12163 (CAY75871.1 GI:283479955), Erwinia amylovora LA637 (CDK23310.1 GI:566688504), Erwinia amylovora LA636 (CDK19939.1 GI:565455063), Erwinia amylovora LA635 (CDK16572.1 GI:565423714), Erwinia piriflorinigrans CFBP 5888 (CCG88689.1 GI:560105768), Erwinia amylovora MR1 (CCP08595.1 GI:478726734), Erwinia amylovora Ea644 (CCP04532.1 GI:478723237), Erwinia amylovora UPN527 (CCP00585.1 GI:478719346), Erwinia amylovora NBRC 12687=CFBP 1232 (00095265.1 GI:478715307), Erwinia amylovora 01SFR-BO (00091470.1 GI:478711918), Erwinia amylovora CFBP 2585 (00087679.1 GI:478708144), Erwinia amylovora Ea266 (00083917.1 GI:478703932), Erwinia amylovora Ea356 (00080113.1 GI:478700098), Erwinia amylovora ACW56400 (EKV52685.1 GI:426274945), or Erwinia amylovora ATCC BAA-2158 (CBX82136.1 GI:312173882).

In particular, E₄ᵢᵢ, or E₄ᵢᵥ is selected from the group consisting of DesD from Streptomyces coelicolor, DesD from Streptomyces violaceoruber, DesD from Streptomyces pilosus, lucA/lucC from Tenacibaculum mesophilum, C-terminal domains of the DesC proteins from Corynebacterium xerosis, C-terminal domains of the DfoC proteins from Erwinia amylovora or Pantoea agglomerans. More in particular, the E₄ᵢᵢ, or E₄ᵢᵥ is selected from the group consisting of Streptomyces coelicolor (NUV53721.1), Streptomyces violaceoruber, Streptomyces pilosus (WP_189555973.1), Erwinia amylovora (WP_004160308.1), Pantoea agglomerans (AWD37890.1) and Corynebacterium xerosis (SLM95104.1 GI:1188028261), and Even more in particular, the E₄ or E₄ comprises at least 70% sequence identity relative to SEQ ID NO:6 (E₄ₐ), SEQ ID NO:18 (E_{4b}), SEQ ID NO:28 (E_{4c}), C-terminal domain of SEQ ID NO:34 (E_{4d}), SEQ ID NO:40 (E₄ₑ) or SEQ ID NO:46 (E_{4f}).

The accession numbers stated in connection with the present invention mentioned throughout this specification correspond to the NCBI ProteinBank database entries with the date 07.02.2022; as a rule, the version number of the entry is identified here by “numerals” such as for example “0.1”.

In one example, E₄ᵢ, E₄ᵢᵢ, E₄ᵢᵢᵢ and E₄ᵢᵥ are the same enzyme in the cell and therefore, a mixture of bisucaberin, bisucaberin B and desferrioxamine E, H, X1 and X2 may be produced. The amount of each compound produced may vary in each reaction depending on the enzyme from the list of E₄ᵢ, E₄ᵢᵢ, E₄ᵢᵢᵢ and E₄ᵢᵥ used, and/or the conditions in which the reaction is carried out. In one example, when E₃ is fused with E₄ (E₄ᵢᵢ, and E₄ᵢᵥ) more desferrioxamine E compared to bisucaberin may be produced as shown in Fujita M J, Mol. Biosyst., 8, 482-485 (2012) and Fujita M J, Biosci. Biotechnol. Biochem., 77 (12), 2467-2472, 2013.

In particular, the cell is further genetically modified or has a further genetic modification to increase activity relative to a wild-type cell of at least one E₄ enzyme selected from the group consisting of:
desferrioxamine synthetase (EC 6.3.-.-) (E₄ᵢ) capable of converting N5-aminopentyl-N-(hydroxyl)-succinamic acid to desferrioxamine B or H; and
bisucaberin synthetase (EC 6.3.-.-) (E₄ᵢᵢᵢ) capable of converting N5-aminopentyl-N-(hydroxyl)-succinamic acid to bisucaberin B
   to produce at least one compound of Formula II:

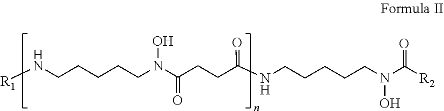

n=1-3
R₁═H or COCH₃ or CH₂CH₂COX with X═OH or O—
R₂═CH₃ or CH₂CH₂COX with X═OH or O—.

In particular, n is 2 or 3.

The compound having structural Formula II according to any aspect of the present invention may be a desferrioxamine and/or an analog thereof. In particular, the compound may be desferrioxamine B with formula II:

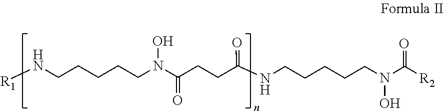

where
n=2, R₁═H and R₂═CH₃.

More in particular, desferrioxamine B has the following formula.

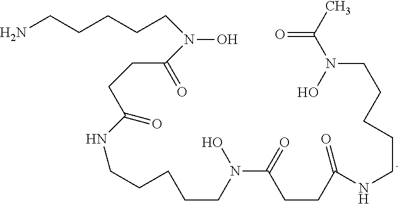 [SMILES: CC(=O)N(O)CCCCCNC(=O)CCC(=O)N(O)CCCCCNC(=O)CCC(=O)N(O)CCCCCN]

In one example, the compound may be desferrioxamine H with formula II:

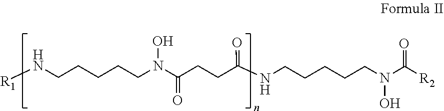

where
n=2, R₁═H and R₂═CH₂CH₂COX with X═OH or O—.

An analog, a structural analog, also known as a chemical analog of desferrioxamine, has a structure that falls within the Formula II and is similar to desferrioxamine, but differs from desferrioxamine in respect to a certain component. For example, the analog of desferrioxamine may differ in one or more atoms, functional groups, or substructures, which are replaced with other atoms, groups, or substructures. Structural analogs are often isoelectronic.

Examples of lysine decarboxylase (E₁), cadaverine N5-monooxygenase (E₂), N5-Aminopentyl-N-(hydroxy)-succinamic acid synthase (E₃), desferrioxamine/bisucaberin synthetase (E₄) and any other enzymes mentioned according to any aspect of the present invention, also include proteins having the same amino acid sequences as those described above except that one or several amino acids are substituted, deleted, inserted and/or added, as long as their functions are maintained. The term “several” herein means normally about 1 to 7, particularly about 1 to 5, more particularly about 1 to 2. Each of the of lysine decarboxylase (E₁), cadaverine N5-monooxygenase (E₂), N5-Aminopentyl-N-(hydroxy)-succinamic acid synthase (E₃), desferrioxamine/bisucaberin synthetase (E₄) and any other enzymes mentioned according to any aspect of the present invention may be a protein having an amino acid sequence with a sequence identity of normally not less than 70, 75, 80, 85%, particularly not less than 90%, more particularly not less than 95% to the original amino acid sequence, as long as its functions is maintained.

The substitution(s), deletion(s), insertion(s) and/or addition(s) in the amino acid sequence described above is/are particularly a conservative substitution(s). Examples of conservative substitution of the original amino acid for another amino acid include substitution of Ala for Ser or Thr; substitution of Arg for Gln, H is or Lys; substitution of Asn for Glu, Gln, Lys, H is or Asp; substitution of Asp for Asn, Glu or Gln; substitution of Cys for Ser or Ala; substitution of Gln for Asn, Glu, Lys, H is, Asp or Arg; substitution of Glu for Asn, Gln, Lys or Asp; substitution of Gly for Pro; substitution of H is for Asn, Lys, Gln, Arg or Tyr; substitution of Ile for Leu, Met, Val or Phe; substitution of Leu for Ile, Met, Val or Phe; substitution of Lys for Asn, Glu, Gln, His or Arg; substitution of Met for Ile, Leu, Val or Phe; substitution of Phe for Trp, Tyr, Met, Ile or Leu; substitution of Ser for Thr or Ala; substitution of Thr for Ser or Ala; substitution of Trp for Phe or Tyr; substitution of Tyr for His, Phe or Trp; and substitution of Val for Met, Ile or Leu.

In particular, the cell according to any aspect of the present invention, may be genetically modified or may comprise a genetic modification to comprise an increased expression relative to its wild-type cell of at least two other enzymes selected from the group consisting of E₁, E₂, and E₃. In one example, the cell has increased activity of E₁, and E₂ or E₁, and E₃ or E₂, and E₃. More in particular, the cell according to any aspect of the present invention may be genetically modified or comprise a genetic modification to comprise an increased expression or activity relative to its wild-type cell of enzymes E₁, E₂, and E₃. Even more in particular, the cell according to any aspect of the present invention may be genetically modified or comprise a genetic modification to comprise an increased activity relative to its wild-type cell of E₄, E₁, and E₂ or E₄, E₁, and E₃ or E₄, E₂, and E₃.

Even more in particular, the cell according to any aspect of the present invention is genetically modified or comprises a genetic modification to comprise an increased expression relative to its wild-type cell of all four enzymes E₁, E₂, E₃ and E₄.

The cell according to any aspect of the present invention is further genetically modified or comprises a further genetic modification to increase production of L-lysine and/or cadaverine. In particular, the cell has increased intracellular production of L-lysine and/or cadaverine. More in particular, the further genetic modification in the cell according to any aspect of the present invention results in increased production of lysine and includes an increase or decrease in expression relative to the wild-type cell of at least one of the following enzymes E₆-E₁₉. More in particular, the further genetic modification according to any aspect of the present invention includes an increase in expression of at least one of the following enzymes:
pyruvate carboxylase (EC 6.4.1.1) (E₆),
aspartate amino transferase (EC 2.6.1.1) (E₇)
aspartate kinase, particularly feedback resistant aspartate kinase (EC 2.7.2.4) (E₈),
aspartate semialdehyde dehydrogenase (EC 1.2.1.11) (E₉),
dihydrodipicolinate synthase (EC 4.3.3.7) (E₁₀),
dihydrodipicolinate reductase (EC 30 1.17.1.8) (E₁₁),
diaminopimelate dehydrogenase (EC 1.4.1.16) (E₁₂),
diaminopimelate epimerase (EC 5.1.1.7) (E₁₃),
diaminopimelate decarboxylase (EC 4.1.1.20) (E₁₄),
N-succinyl-aminoketopimelate aminotranferase (EC 2.6.1.17) (E₁₇),
2,3,4,5-tetrahydropyridine-2,6-dicarboxylate N-succinyltransferase (EC 2.3.1.117) (E₁₈), and/or
Succinyl-diaminopimelate desuccinylase (EC 3.5.1.18) (E₁₉);
   and/or a decrease in expression of at least one of the following enzymes:
phosphoenolpyruvate carboxykinase (EC 4.1.1.32) (E₁₅), and/or
homoserine dehydrogenase (EC 1.1.1.3) (E₁₆).

In one example, E₆ may be encoded by gene pyc P458S of Corynebacterium glutamicum disclosed at least in WO1999018228 or EP2107128. In one example, E₇ may be encoded by gene aspB of Corynebacterium glutamicum disclosed at least in EP0219027 or WO2008033001. In one example, E₈ may be a variant T3111 and may be encoded by gene lysC of Corynebacterium glutamicum disclosed at least in U.S. Pat. No. 6,893,848. In one example, E₉ may be encoded by gene asd of Corynebacterium glutamicum disclosed at least in EP0387527 or WO2008033001. In one example, E₁₀ may be encoded by gene dapA of Corynebacterium glutamicum disclosed at least in EP0197335. In one example, E₁₁ may be encoded by gene dapB of Corynebacterium glutamicum disclosed at least in U.S. Pat. No. 8,637,295 or EP0841395. In one example, E₁₂ may be encoded by gene ddh of Corynebacterium glutamicum described at least in EP0811682. In one example, E₁₃ may be encoded by gene dapF of Corynebacterium glutamicum described at least in U.S. Pat. No. 6,670,156. In one example, E₁₄ may be encoded by gene lysA of Corynebacterium glutamicum described at least in EP0811682. In one example, E₁₅ may be encoded by gene pck of Corynebacterium glutamicum. In one example, E₁₆ may be encoded by gene homV59A of Corynebacterium glutamicum. In one example, E₁₇ may be encoded by gene dapC of Corynebacterium glutamicum. In one example, E₁₈ may be encoded by gene dapD of Corynebacterium glutamicum. In one example, E₁₉ may be encoded by gene dapE of Corynebacterium glutamicum.

In another example, the cell according to any aspect of the present invention may be genetically modified to increase the expression of at least 2, 3, 4, 5, 6, 7, 8, 9, 10, 11, 12, 13, or 14 enzymes selected from the group consisting of E₆ to E₁₉.

In yet another example, the cell according to any aspect of the present invention may be genetically modified to increase the expression of at least pyruvate carboxylase (EC 6.4.1.1) (E₆), and aspartate kinase (EC 2.7.2.4) (E₈) as disclosed at least in JP6219481B2 to increase lysine production.

In a further example, the cell according to any aspect of the present invention may be genetically modified to increase the expression of at least dihydrodipicolinate synthase (EC 4.3.3.7) (E₁₀), aspartate kinase, (EC 2.7.2.4) (E₈), dihydrodipicolinate reductase (EC 30 1.17.1.8) (E₁₁), phosphoenolpyruvate carboxykinase (EC 4.1.1.32) (E₁₆), and aspartate semialdehyde dehydrogenase (EC 1.2.1.11) (E₉) as disclosed in U.S. Pat. No. 8,062,869B2.

In one example, the cell according to any aspect of the present invention may be genetically modified to increase the expression of at least aspartate semialdehyde dehydrogenase (EC 1.2.1.11) (E₉), dihydrodipicolinate synthase (EC 4.3.3.7) (E₁₀) and dihydrodipicolinate reductase (EC 30 1.17.1.8) (E₁₁) as disclosed at least in JP5486029B2. The cell may further be genetically modified to increase the expression of diaminopimelate dehydrogenase (EC 1.4.1.16) (E₁₂), and/or diaminopimelate decarboxylase (EC 4.1.1.20) (E₁₅).

In a further example, the cell according to any aspect of the present invention may be genetically modified to increase the expression of pyruvate carboxylase (EC 6.4.1.1) (E₆), aspartate kinase (EC 2.7.2.4) (E₈), aspartate semialdehyde dehydrogenase (EC 1.2.1.11) (E₉), dihydrodipicolinate synthase (EC 4.3.3.7) (E₁₀), dihydrodipicolinate reductase (EC 30 1.17.1.8) (E₁₁), diaminopimelate dehydrogenase (EC 1.4.1.16) (E₁₂), and/or diaminopimelate decarboxylase (EC 4.1.1.20) (E₁₄) and to decrease the expression of phosphoenolpyruvate carboxykinase (EC 4.1.1.32) (E₁₅), and/or homoserine dehydrogenase (EC 1.1.1.3) (E₁₆). Examples of sequences of E₆-E₁₉ and means of increasing and decreasing the expression of the relevant enzymes is shown provided at least in U.S. Pat. No. 8,637,295, US2011039313, EP1725672 and EP1320593.

Further, the examples use strain C. glutamicum DM1933 and the construction of which is at least disclosed in Blomberg et al., 2009 (doi:10.1128/AEM.01844-08). This method may then be used to produce any cell that may have increased lysine production compared to the wild type cell.

According to another aspect of the present invention, there is provided a method of producing at least one compound having structural Formula I from at least one simple carbon source:

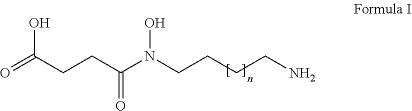

where n is 2.
(a) contacting the cell according to any aspect of the present invention with at least one simple carbon source, wherein the simple carbon source is selected from the group consisting of glucose, sucrose, xylose, arabinose, mannose and glycerol.

According to another aspect of the present invention, there is provided a use of the cell according to any aspect of the present invention for producing at least one compound having structural Formula I from at least one simple carbon source:

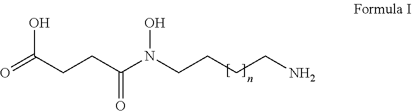

where n is 2, and wherein the simple carbon source is selected from the group consisting of glucose, sucrose, xylose, arabinose, mannose and glycerol.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Construction of a C. glutamicum Expression Vector for the Streptomyces coelicolor N-Succinyl-N-Hydroxycadaverine Biosynthesis Genes desBCD_Sco

For the heterologous expression of the desB gene (SEQ ID NO: 1), desC gene (SEQ ID NO: 2) and desD gene (SEQ ID NO: 3) from Streptomyces coelicolor CAI-140 the plasmid pXMJ19{Ptac}[desBCD_Sco]{T} was constructed. The synthetic operon consisting of desB_Sco encoding a lysine N(6)-hydroxylase/L-ornithine N(5)-oxygenase family protein (DesB, EC 1.14.13.-, SEQ ID NO: 4), desC_Sco encoding an acetyltransferase (DesC, EC 2.3.-.-, SEQ ID NO: 5) and desD_Sco encoding a lucA/lucC family siderophore biosynthesis protein (DesD, EC 6.3.-.-, SEQ ID NO: 6), respectively, was cloned under the control of the IPTG inducible promoter Ptac into the E. coli/C. glutamicum shuttle vector pXMJ19. Downstream of the synthetic operon a terminator sequence is located. The complete synthetic operon including the terminator sequence (3732 bp, SEQ ID NO: 7) was ordered for gene synthesis from Eurofins Genomics Germany GmbH (Ebersberg, Germany). The E. coli/C. glutamicum shuttle vector pXMJ19 carries a pUC origin of replication for E. coli and a pBL1 origin of replication for the replication in C. glutamicum ATCC 13032. For cloning the synthetic operon was cut with the restriction enzyme HindIII and ligated into pXMJ19 cut with the same enzyme. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}[desBCD_Sco]{T}(SEQ ID NO: 8, See Table 2 below).

### Example 2

Construction of a C. glutamicum Expression Vector for the Streptomyces coelicolor N-Succinyl-N-Hydroxycadaverine Biosynthesis Genes desBCD_Sco Codon-Optimized for Expression in C. glutamicum

For the heterologous expression of the desB gene (SEQ ID NO: 1), desC gene (SEQ ID NO: 2) and desD gene (SEQ ID NO: 3) from Streptomyces coelicolor CA-140 the plasmid pXMJ19{Ptac}{RBSopt}[desBCD_Sco(co_Cg)]{T} was constructed. The synthetic operon consisting of desB_Sco encoding a lysine N(6)-hydroxylase/L-ornithine N(5)-oxygenase family protein (DesB, EC 1.14.13.-, SEQ ID NO: 4), desC_Sco encoding an acetyltransferase (DesC, EC 2.3.-.-, SEQ ID NO: 5) and desD_Sco encoding a lucA/lucC family siderophore biosynthesis protein (DesD, EC 6.3.-.-, SEQ ID NO: 6), respectively, was cloned under the control of the IPTG inducible promoter Ptac into the E. coli/C. glutamicum shuttle vector pXMJ19. Upstream of the operon an optimized ribosome binding site (RBS) for C. glutamicum was added and downstream of the synthetic operon a terminator sequence is located. The complete synthetic operon including the RBS and the terminator sequence (3779 bp, SEQ ID NO: 9) was ordered for gene synthesis from Eurofins Genomics Germany GmbH (Ebersberg, Germany) and the DNA sequence of the gene fragment was codon-optimized for expression in C. glutamicum ATCC 13032. The E. coli/C. glutamicum shuttle vector pXMJ19 carries a pUC origin of replication for E. coli and a pBL1 origin of replication for the replication in C. glutamicum ATCC 13032. For cloning the synthetic operon was cut with the restriction enzyme HindIII and ligated into pXMJ19 cut with the same enzyme. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}{RBSopt}[desBCD_Sco(co_Cg)]{T}(SEQ ID NO: 10, see Table 2 below).

### Example 3

Construction of a C. glutamicum Expression Vector for the Streptomyces violaceoruber N-Succinyl-N-Hydroxycadaverine Biosynthesis Genes desABCD_Svi

For the heterologous expression of the desA gene (SEQ ID NO: 11), desB gene (SEQ ID NO: 12), desC gene (SEQ ID NO: 13) and desD gene (SEQ ID NO: 14) from Streptomyces violaceoruber A3(2) the plasmid pXMJ19{Ptac}[desABCD_Svi] was constructed. The complete operon consisting of desA encoding a lysine decarboxylase (DesA, EC 4.1.1.18, SEQ ID NO: 15, desB_Sco encoding a lysine N(6)-hydroxylase/L-ornithine N(5)-oxygenase family protein (DesB, EC 1.14.13.-, SEQ ID NO: 16), desC_Sco encoding an acetyltransferase (DesC, EC 2.3.-.-, SEQ ID NO: 17) and desD_Sco encoding a lucA/lucC family siderophore biosynthesis protein (DesD, EC 6.3.-.-, SEQ ID NO: 18), respectively, was cloned under the control of the IPTG inducible promoter Ptac into the E. coli/C. glutamicum shuttle vector pXMJ19. The complete operon was amplified via PCR using the primer pair MW22_001fw/MW22_002rv (SEQ ID NO: 56, SEQ ID NO: 57, see Table 1 below) and genomic DNA from Streptomyces violaceoruber A3(2) as template. The E. coli/C. glutamicum shuttle vector pXMJ19 carries a pUC origin of replication for E. coli and a pBL1 origin of replication for the replication in C. glutamicum ATCC 13032. The PCR product (5120 bp, SEQ ID NO: 19) was cloned into the vector pXMJ19 using the restriction site HindIII and NEBuilder® HiFi DNA Assembly Cloning Kit from New England BioLabs Inc., Ipswich, USA, Cat. No. E5520. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}[desABCD_Svi](SEQ ID NO: 20, see Table 2 below).

TABLE 1
Primer list

| Name | 5′-3′ | SEQ IDs |
|---|---|---|
| MW22_001fw | ACAGGAAACAGAATTAATTAAGCTTCCCCACCGGAGGA | SEQ ID NO: 56 |
| | CCCCCCCATG | |
| MW22_002rv | AGTCGACCTGCAGGCATGCAAGCTTCCGGTTGGCTACC | SEQ ID NO: 57 |
| | GGCCTGCCAGG | |
| MW22_006fw | ACAGGAAACAGAATTAATTAAGCTTTGCAGGAAGGAGA | SEQ ID NO: 58 |
| | TATAGATATGCGCTCGCACCTGCTCAACGACACCACCG | |
| | CGGAGCACTACCGCCGCTCCGTGAC | |
| MW22_005rv | AGTCGACCTGCAGGCATGCAAGCTTTTCGTGGGTCAGA | SEQ ID NO: 59 |
| | ACTCCGCGAGGGGGTTC | |
| MW_21_80_fw | ACAGCTATGACATGATTACGGACGGCGAATTCCTCCTC | SEQ ID NO: 60 |
| | GTTG | |
| MW_21_81_rv | GGCGCGCCTCCTGACATCCTGCTTGCAAAG | SEQ ID NO: 61 |
| MW_21_82_fw | GCAGGATGTCAGGAGGCGCGCCTCCACAGTTTTCCCGG | SEQ ID NO: 62 |
| | TTCATCTTG | |
| MW_21_83_rv | ATCCCCGGGTACCGAGCTCGAATTCATCGTCTTCCTTG | SEQ ID NO: 63 |
| | GTCTGCAGTTAG | |
| MW_21_93_fw | GCAAGCAGGATGTCAGGAGGACCGGTGTTTTAG | SEQ ID NO: 64 |
| MW_21_94_rv | AACCGGGAAAACTGTGGAGGCGGAT | SEQ ID NO: 65 |
| MW22_013fw | TCCTAGAGCCAGTCCGCGATCGCCTCCGCTTCGACGGT | SEQ ID NO: 66 |
| | CAC | |
| MW22_014rv | ATTCGAGCTCGGTACCCGGGTCAGGCGGAGGCGCCGCG | SEQ ID NO: 67 |
| | AGCTG | |

### Example 4

Construction of a C. glutamicum Expression Vector for the Streptomyces pilosus N-Succinyl-N-Hydroxycadaverine Biosynthesis Genes desABCD_Spi

For the heterologous expression of the desA gene (SEQ ID NO: 21), desB gene (SEQ ID NO: 22), desC gene (SEQ ID NO: 23) and desD gene (SEQ ID NO: 24) from Streptomyces pilosus ATCC19797 the plasmid pXMJ19{Ptac}[desABCD_Spi] was constructed. The complete operon consisting of desA encoding a lysine decarboxylase (DesA, EC 4.1.1.18, SEQ ID NO: 25, desB_Sco encoding a lysine N(6)-hydroxylase/L-ornithine N(5)-oxygenase family protein (DesB, EC 1.14.13.-, SEQ ID NO: 26), desC_Sco encoding an acetyltransferase (DesC, EC 2.3.-.-, SEQ ID NO: 27) and desD_Sco encoding a lucA/lucC family siderophore biosynthesis protein (DesD, EC 6.3.-.-, SEQ ID NO: 28), respectively, was cloned under the control of the IPTG inducible promoter Ptac into the E. coli/C. glutamicum shuttle vector pXMJ19. Upstream of the operon an optimized ribosome binding site (RBS) for C. glutamicum was included via primer sequence. The complete operon was amplified via PCR using the primer pair MW22_006fw/MW22_005rv (SEQ ID NO: 58, SEQ ID NO: 59, see Table 1) and genomic DNA from Streptomyces pilosus ATCC19797 as template. The E. coli/C. glutamicum shuttle vector pXMJ19 carries a pUC origin of replication for E. coli and a pBL1 origin of replication for the replication in C. glutamicum ATCC 13032. The PCR product (5102 bp, SEQ ID NO: 29) was cloned into the vector pXMJ19 using the restriction site HindIII and NEBuilder® HiFi DNA Assembly Cloning Kit from New England BioLabs Inc., Ipswich, USA, Cat. No. E5520. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}{RBSopt}[desABCD_Spi](SEQ ID NO: 30, see Table 2 below).

### Example 5

Construction of a C. glutamicum Expression Vector for the Erwinia amylovora N-Succinyl-N-Hydroxycadaverine Biosynthesis Genes dfoAC_Eam Codon-Optimized for Expression in C. glutamicum

For the heterologous expression of the dfoA gene (SEQ ID NO: 31) and dfoC gene (SEQ ID NO: 32) from Erwinia amylovora CFBP1430 the plasmid pXMJ19{Ptac}[dfoAC_Eam(co_Cg)]{T} was constructed. The synthetic operon consisting of dfoA_Eam encoding a lysine N(6)-hydroxylase/L-ornithine N(5)-oxygenase family protein (DfoA, EC 1.14.13.-, SEQ ID NO: 33) and dfoC_Eam encoding an GNAT family N-acetyltransferase (DfoC, EC 2.3.-.-, SEQ ID NO: 34) was cloned under the control of the IPTG inducible promoter Ptac into the E. coli/C. glutamicum shuttle vector pXMJ19. Downstream of the synthetic operon a terminator sequence is located. The complete synthetic operon including the terminator sequence (3830 bp, SEQ ID NO: 35) was ordered for gene synthesis from Eurofins Genomics Germany GmbH (Ebersberg, Germany) and the DNA sequence of the gene fragment was codon-optimized for expression in C. glutamicum ATCC 13032. The E. coli/C. glutamicum shuttle vector pXMJ19 carries a pUC origin of replication for E. coli and a pBL1 origin of replication for the replication in C. glutamicum ATCC 13032. For cloning the synthetic operon was cut with the restriction enzyme HindIII and ligated into pXMJ19 cut with the same enzyme. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}[dfoAC_Eam(co_Cg)]{T}(SEQ ID NO: 36, see Table 2 below).

### Example 6

Construction of a C. glutamicum Expression Vector for the Pantoea agglomerans N-Succinyl-N-Hydroxycadaverine Biosynthesis Genes dfoAC_Pag

For the heterologous expression of the dfoA gene (SEQ ID NO: 37) and dfoC gene (SEQ ID NO: 38) from Pantoea agglomerans strain DC432 the plasmid pXMJ19{Ptac}[dfoAC_Pag]{T} was constructed. The operon consisting of dfoA_Pag encoding a lysine N(6)-hydroxylase/L-ornithine N(5)-oxygenase family protein (DfoA, EC 1.14.13.-, SEQ ID NO: 39) and dfoC_Pag encoding a GNAT family N-acetyltransferase (DfoC, EC 2.3.-.-, SEQ ID NO: 40) was cloned under the control of the IPTG inducible promoter Ptac into the E. coli/C. glutamicum shuttle vector pXMJ19. Downstream of the synthetic operon a terminator sequence is located. The operon including the terminator sequence (3842 bp, SEQ ID NO: 41) was ordered for gene synthesis from Eurofins Genomics Germany GmbH (Ebersberg, Germany). The E. coli/C. glutamicum shuttle vector pXMJ19 carries a pUC origin of replication for E. coli and a pBL1 origin of replication for the replication in C. glutamicum ATCC 13032. For cloning the synthetic operon was cut with the restriction enzyme HindIII and ligated into pXMJ19 cut with the same enzyme. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}[dfoAC_Pag]{T}(SEQ ID NO: 42, see Table 2 below).

### Example 7

Construction of a C. glutamicum Expression Vector for the Corynebacterium xerosis N-Succinyl-N-Hydroxycadaverine Biosynthesis Genes desBC_Cx

For the heterologous expression of the desB gene (SEQ ID NO: 43) and desC gene (SEQ ID NO: 44) from Corynebacterium xerosis the plasmid pXMJ19{Ptac}[desBC_Cx]{T} was constructed. The operon consisting of desB_Cx encoding a siderophore biosynthesis protein, monooxygenase (DesB, EC 1.14.13.-, SEQ ID NO: 45) and desC_Cx encoding a desferrioxamine biosynthesis protein/siderophore synthetase superfamily, group C (DesC, EC 2.3.-.-, SEQ ID NO: 46) was cloned under the control of the IPTG inducible promoter Ptac into the E. coli/C. glutamicum shuttle vector pXMJ19. Downstream of the synthetic operon a terminator sequence is located. The operon including the terminator sequence (3922 bp, SEQ ID NO: 47) was ordered for gene synthesis from Eurofins Genomics Germany GmbH (Ebersberg, Germany). The E. coli/C. glutamicum shuttle vector pXMJ19 carries a pUC origin of replication for E. coli and a pBL1 origin of replication for the replication in C. glutamicum ATCC 13032. For cloning the synthetic operon was cut with the restriction enzyme HindIII and ligated into pXMJ19 cut with the same enzyme. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}[desBC_Cx]{T}(SEQ ID NO: 48, see Table 2 below).

### Example 8

Construction of a C. glutamicum Expression Vector for the Streptomyces coelicolor N-Succinyl-N-Hydroxycadaverine Biosynthesis Genes desBC_Sco Codon-Optimized for Expression in C. glutamicum

For the heterologous expression of the desB gene (SEQ ID NO: 1) and desC gene (SEQ ID NO: 2) from Streptomyces coelicolor CAI-140 the plasmid pXMJ19{Ptac}{RBSopt}[desBC_Sco(co_Cg)]{T} was constructed. The synthetic operon consisting of desB_Sco encoding a lysine N(6)-hydroxylase/L-ornithine N(5)-oxygenase family protein (DesB, EC 1.14.13.-, SEQ ID NO: 4) and desC_Sco encoding an acetyltransferase was cloned under the control of the IPTG inducible promoter Ptac into the E. coli/C. glutamicum shuttle vector pXMJ19. Upstream of the operon an optimized ribosome binding site (RBS) for C. glutamicum was added. For cloning the plasmid pXMJ19{Ptac}{RBSopt}[desBCD_Sco(co_Cg)]{T}(SEQ ID NO: 10, see Table 2 below and Example 2 above) was cut with AsiSI/BamHI so that part of the desB, desC and desD gene were removed. To restore desB and desC the genes were amplified via PCR using the primer pair MW22_013fw/MW22_014rv (SEQ ID NO: 66, SEQ ID NO: 67, see Table 1) using pXMJ19{Ptac}{RBSopt}[desBCD_Sco(co_Cg)]{T} as a template. The E. coli/C. glutamicum shuttle vector pXMJ19 carries a pUC origin of replication for E. coli and a pBL1 origin of replication for the replication in C. glutamicum ATCC 13032. The PCR product (831 bp, SEQ ID NO: 68) was cloned into the vector pXMJ19 using NEBuilder® HiFi DNA Assembly Cloning Kit from New England BioLabs Inc., Ipswich, USA, Cat. No. E₅₅₂₀. The ligation product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target genes was checked by restriction analysis and the authenticity of the introduced DNA fragment was verified by DNA sequencing. The resulting expression vector was named pXMJ19{Ptac}{RBSopt}[desABC_Spi] (SEQ ID NO: 69, see Table 2 below).

TABLE 2
List of C. glutamicum/E. coli expression plasmids

| Plasmid | Cloned gene |
|---|---|
| pXMJ19{Ptac}[desBCD_Sco]{T} | desBCD_Sco |
| pXMJ19{Ptac}{RBSopt}[desBCD_Sco(co_Cg)}{T} | desBCD_Sco(co_Cg) |
| pXMJ19{Ptac}[desABCD_Svi] | desABCD_Svi |
| pXMJ19{Ptac}{RBSopt}[desABCD_Spi] | desABCD_Spi |
| pXMJ19{Ptac}[dfoAC_Eam(co_Cg)]{T} | dfoAC_Eam |
| pXMJ19{Ptac}[dfoAC_Pag]{T} | dfoAC_Pag |
| pXMJ19{Ptac}[desBC_Cx]{T} | desBC_Cx |
| pXMJ19{Ptac}{RBSopt}[desBC_Sco(co_Cg)]{T} | desBC_Sco(co_Cg) |

### Example 9

Construction of a C. glutamicum Based Cadaverine Producer by Introduction of L-Lysine Decarboxylase ldcC from E. coli into the L-Lysine Producer Strain C. glutamicum DM1933 which Produces More Lysine than the Wild-Type Cell

For construction of a C. glutamicum based cadaverine producer the E. coli ldcC gene (SEQ ID NO: 49) encoding a L-lysine decarboxylase (LdcC, EC 4.1.1.18, SEQ ID NO: 50) was integrated into the genome of the lysine producer C. glutamicum DM1933. The detailed construction of DM1933 is described in Blomberg et al., 2009 (doi:10.1128/AEM.01844-08). For the integration of the ldcC gene the plasmid pK18mobsacB KI {Ptuf}[ldcC_Ec(co_Cg)] was constructed. The ldcC gene was integrated into the intergenic region between ORF NCgI0013 and ORF NCgI0014 and was cloned under the control of the constitutive C. glutamicum promoter P_{tuf}. The {Ptuf}[ldcC_Ec(co_Cg)] fusion product (SEQ ID NO: 51) was ordered for gene synthesis from Eurofins Genomics Germany GmbH (Ebersberg, Germany).

In the first cloning step the two flanking regions of the chromosomal none-coding region between NCgI0013 and NCgI0014 were amplified by PCR using the primer pairs MW_21_80/MW_21_81 (SEQ ID NO: 60, SEQ ID NO: 61) and MW_21_82/MW_21_83 (SEQ ID NO: 62, SEQ ID NO: 63, see Table 1 above), resulting in fragments HomA (1046 bp, SEQ ID NO: 52) and HomB (1028 bp, SEQ ID NO: 53). The two fragments were cloned into the vector pK18mobsacB (Schäfer et al., 1994, DOI: 10.1016/0378-1119(94)90324-7) using the restriction site EcoRI and NEBuilder® HiFi DNA Assembly Cloning Kit from New England BioLabs Inc., Ipswich, USA, Cat. No. E5520. Additionally, an AscI restriction site was introduced between HomA and HomB via primers MW_21_81/MW_21_82. The assembled product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target gene was checked by restriction analysis and the authenticity of the introduced DNA fragment was verified by DNA sequencing. The resulting expression vector was named pK18mobsacB[KI NCgI0013 locus](SEQ ID NO: 54).

In the second cloning step the {Ptuf}[ldcC_Ec(co_Cg)] fusion product (SEQ ID NO: 51, 2433 bp) was amplified via PCR using the primer pair MW_21_93/MW_21_94 (SEQ ID NO: 64, SEQ ID NO: 65) and cloned into the vector pK18mobsacB[KI NCgI0013 locus](SEQ ID NO: 54) using the restriction sites AscI and NEBuilder® HiFi DNA Assembly Cloning Kit from New England BioLabs Inc., Ipswich, USA, Cat. No. E5520. The assembled product was transformed into 10-beta electrocomponent E. coli cells (New England BioLabs Inc., Ipswich, USA, Cat. No. C3020K). Procedure of cloning and transformation were carried out according to manufacturer's manual. The correct insertion of the target gene was checked by restriction analysis and the authenticity of the introduced DNA fragment was verified by DNA sequencing. The resulting knock-in plasmid was named pK18mobsacB KI {Ptuf}[ldcC_Ec(co_Cg)](SEQ ID NO: 55).

This plasmid was transformed into C. glutamicum DM1933 via elctroporation. By application of the method described by Schäfer et al. 1994 (DOI: 10.1016/0378-1119(94)90324-7), the gene Idc_Ec(co_Cg) under the control of the promoter Ptu was integrated into the chromosome of C. glutamicum DM1933 via homologous recombination (double crossover), resulting in C. glutamicum DM1933 int.NCGI0013/0014::{Ptuf}[ldcC_Ec(coCg)].

### Example 10

Construction of a C. glutamicum Based N-Succinyl-N-Hydroxycadaverine Producer

For the construction of a C. glutamicum based N-succinyl-N-hydroxycadaverine producer, the plasmids described in Examples 2-4, 6, 7 and 8 and listed in Table 2 were transformed into C. glutamicum DM1933 int.NCGI0013/0014::{Ptuf}[ldcC_Ec(coCg)] by means of electroporation. The cells were plated onto LB-agar plates supplemented with chloramphenicol (7.5 mg/L). Transformants were checked for the presence of the correct plasmid by plasmid preparation and analytic restriction analysis. The resulting strains are listed in Table 3 below.

TABLE 3
List of C. glutamicum based desferrioxamine producer strains

| Plasmid | Recipient strain | Resulting strain |
|---|---|---|
| pXMJ19{Ptac}{RBSopt}[desBCD_{—} | C. glutamicum DM1933 | C. glutamicum DM1933 |
| Sco(co_Cg)]{T} | int.NCGI0013/0014::{Ptuf}[IdcC_Ec(coCg)] | int.NCGI0013/0014::{Ptuf}[IdcC_Ec(coCg)] |
| | | pXMJ19{Ptac}{RBSopt}[desBCD_Sco(co_Cg)]{T} |
| pXMJ19{Ptac}[desABCD_Svi] | C. glutamicum DM1933 | C. glutamicum DM1933 |
| | int.NCGI0013/0014::{Ptuf}[IdcC_Ec(coCg)] | int.NCGI0013/0014::{Ptuf}[IdcC_Ec(coCg)] |
| | | pXMJ19{Ptac}[desABCD_Svi] |
| pXMJ19{Ptac}{RBSopt}[desABCD_Spi] | C. glutamicum DM1933 | C. glutamicum DM1933 |
| | int.NCGI0013/0014::{Ptuf}[IdcC_Ec(coCg)] | int.NCGI0013/0014::{Ptuf}[IdcC_Ec(coCg)] |
| | | pXMJ19{Ptac}{RBSopt}[desABCD_Spi] |
| pXMJ19{Ptac}[dfoAC_Pag]{T} | C. glutamicum DM1933 | C. glutamicum DM1933 |
| | int.NCGI0013/0014::{Ptuf}[IdcC_Ec(coCg)] | int.NCGI0013/0014::{Ptuf}[IdcC_Ec(coCg)] |
| | | pXMJ19{Ptac}[dfoAC_Pag]{T} |
| pXMJ19{Ptac}[desBC_Cx]{T} | C. glutamicum DM1933 | C. glutamicum DM1933 |
| | int.NCGI0013/0014::{Ptuf}[IdcC_Ec(coCg)] | int.NCGI0013/0014::{Ptuf}[IdcC_Ec(coCg)] |
| | | pXMJ19{Ptac}[desBC_Cx]{T} |
| pXMJ19{Ptac}{RBSopt}[desBC_{—} | C. glutamicum DM1933 | C. glutamicum DM1933 |
| Sco(co_Cg)]{T} | int.NCGI0013/0014::{Ptuf}[IdcC_Ec(coCg)] | int.NCGI0013/0014::{Ptuf}[IdcC_Ec(coCg)] |
| | | pXMJ19{Ptac}{RBSopt}[desBC_Sco(co_Cg)]{T} |

### Example 11

Construction of a E. coli-Based N-Succinyl-N-Hydroxycadaverine Producer

For the construction of a E. coli based N-succinyl-N-hydroxycadaverine producer, the plasmids described in Examples 1, 2, 5 and 6 and listed in Table 2 were transformed into E. coli W3110 by means of electroporation. The cells were plated onto LB-agar plates supplemented with chloramphenicol (20 mg/L). Transformants were checked for the presence of the correct plasmid by plasmid preparation and analytic restriction analysis. The resulting strains are listed in Table 4.

TABLE 4
List of E. coli based desferrioxamine producer strains

| Plasmid | Recipient strain | Resulting strain |
|---|---|---|
| pXMJ19{Ptac}[desBCD_Sco]{T} | E. coli W3110 | E. coli W3110 |
| | | pXMJ19{Ptac}[desBCD_Sco]{T} |
| pXMJ19{Ptac}{RBSopt}[desBCD_Sco(co_Cg)]{T} | E. coli W3110 | E. coli W3110 |
| | | pXMJ19{Ptac}{RBSopt}[desBCD_Sco(co_Cg)]{T} |
| pXMJ19{Ptac}[dfoAC_Eam(co_Cg)]{T} | E. coli W3110 | E. coli W3110 |
| | | pXMJ19{Ptac}[dfoAC_Eam(co_Cg)]{T} |
| pXMJ19{Ptac}[dfoAC_Pag]{T} | E. coli W3110 | E. coli W3110 |
| | | pXMJ19{Ptac}[dfoAC_Pag]{T} |

### Example 12

Production of N-Succinyl-N-Hydroxycadaverine with C. glutamicum Derivatives

To produce N-succinyl-N-hydroxycadaverine 10 ml BHI medium (GranuCult™ BHI (Brain Heart Infusion) broth, Merck, Darmstadt, Germany, Cat-No: 1.10493.0500) supplemented with chloramphenicol (7.5 mg/L) in 100 ml baffled shake flasks were inoculated with 0.1 ml of a stock culture and incubated for 16 h at 30° C. and 200 rpm. The pre-culture was harvested by centrifugation (10 min, 4000 g, 4° C.) and the pellet was washed twice with 10 ml 0.9% (w/v) NaCl. For the main culture, a FlowerPlate with pH and dissolved oxygen optodes (48 well MTP, flower, Beckman Coulter Life Sciences, Baesweiler, Germany, Cat.-No: M2P-MTP-48-BOH1) containing 0.7 ml CGXII medium (15 g/L glucose, 20 g/L (NH₄)₂SO₄, 5 g/L urea, 1 g/L K₂HPO₄, 1 g/L KH₂PO₄, 0.25 g/L MgSO₄×7H₂O, 42 g/L MOPS, 13.2 mg/L CaCl₂), 0.2 mg/L biotin, 30 mg/L protocatechuic acid, trace element solution: 10 g/L FeSO₄×7H₂O, 10 g/L MnSO₄×H₂O, 1 g/L ZnSO₄×7H₂O, 0.2 g/L CuSO₄, 20 mg/L NiCl₂×6H₂O, pH 7) supplemented with chloramphenicol (7.5 mg/L) in each well was inoculated with the preculture to reach a start OD₆₀₀ of 0.5. The main culture was incubated for 24 h at 30° C. and 1400 rpm and a relative humidity (85%) in a BioLector I system (Beckman Coulter Life Sciences, Baesweiler, Germany). At the beginning of exponential phase, the expression of the target genes was induced with 0.5 mM IPTG. At the end of cultivation, the cells were harvested, and supernatants were sterile-filtered with an 0.2 μm PVDF filter and stored at −20° C. before analysis. N-succinyl-N-hydroxycadaverine concentration of all strains was analyzed via LC-UV-MS (see Example 14). In the supernatants of all strains N-succinyl-N-hydroxycadaverine could be detected.

### Example 13

Production of N-Succinyl-N-Hydroxycadaverine with E. coli Derivatives

To produce N-succinyl-N-hydroxycadaverine 10 ml LB medium (Carl Roth, Karlsruhe, Germany, Cat-No: X968.1) supplemented with chloramphenicol (20 mg/L) in 100 ml baffled shake flasks were inoculated with 0.1 ml of a stock culture and incubated for 16 h at 37° C. and 200 rpm. For the main culture, a FlowerPlate with pH and dissolved oxygen optodes (48 well MTP, flower, Beckman Coulter Life Sciences, Baesweiler, Germany, Cat-No: M2P-MTP-48-BOH1) containing 0.7 ml LB medium (Carl Roth, Karlsruhe, Germany, Cat-No: X968.1), buffered with 100 mM MOPS, pH 7.2 and supplemented with chloramphenicol (20 mg/L) in each well was inoculated with the preculture to reach a start OD₆₀₀ of 0.1. The main culture was incubated for 24 h at 37° C. and 1400 rpm and a relative humidity (85%) in a BioLector I system (Beckman Coulter Life Sciences, Baesweiler, Germany). At the beginning of exponential phase, the expression of the target genes was induced with 0.5 mM IPTG. At the end of cultivation, the cells were harvested, and supernatants were sterile-filtered with an 0.2 μm PVDF filter and stored at −20° C. before analysis. N-succinyl-N-hydroxycadaverine concentration of all strains was analyzed via LC-UV-MS (see Example 14). In the supernatant of all strains N-succinyl-N-hydroxycadaverine could be detected.

### Example 14

### HPLC-Based Quantification of N-Succinyl-N-Hydroxycadaverine

Quantification of N-succinyl-N-hydroxycadaverine was carried out by means of HPLC. Before analysis samples were centrifuged for 5 min at 16100 g and filtrated using a 0.22 μm PVDF filter. 20 μl of the filtrated supernatant were mixed with 80 μl ferric ammonium sulfate solution and filled into a HPLC vial. Samples were stored at −20° C. before measurement.

For the detection and quantification of N-succinyl-N-hydroxycadaverine a DAD detector (198 and 430 nm) was used. The measurement was carried out by means of Agilent Technologies 1200 Series (Santa Clara, Calif., USA) and a XB-C18 column (100 Å, 4.6×100 mm, 2.6 μm, Phenomenex Kinetex). The injection volume was 5 μl and the run time was 25 min at a flow rate of 0.8 ml/min. Mobile phase A: 1 L pure water, 1 ml formic acid, mobile phase B: 1 L acetonitrile, 1 ml formic acid. The column temperature was 40° C. The identification of N-succinyl-N-hydroxycadaverine was done using retention time and corresponding mass spectrum.

### Gradient:

| t [min] | [%] A | [%] B | Flow [ml/min] | Max pressure [bar] |
|---|---|---|---|---|
| 0.0 | 95 | 5 | 0.8 | 400 |
| 5.0 | 95 | 5 | 0.8 | 400 |
| 13.0 | 70 | 30 | 0.8 | 400 |
| 15.0 | 2 | 98 | 0.8 | 400 |
| 20.0 | 2 | 98 | 0.8 | 400 |
| 20.01 | 95 | 5 | 0.8 | 400 |
| 25.0 | 95 | 5 | 0.8 | 400 |

For analytes with a concentration below the limit of quantification (LOQ), the identification was performed by means of HPLC/ESI-MS-MS. The Multiple reaction monitoring mode (MRM) of a Triple Quadrupol Mass Spectrometer (Agilent 6410B, Santa Clara, Calif., USA) was used for these measurements.

1. A recombinant microbial cell for producing at least one compound of Formula I from at least one simple carbon source: 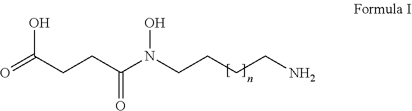
where n is 2; and
wherein the simple carbon source is selected from the group consisting of glucose, sucrose, xylose, arabinose, mannose, glycerol and combinations thereof and wherein the cell comprises a further genetic modification to increase production of L-lysine from at least one of the simple carbon sources.

2. The cell according to claim 1, wherein the cell comprises a genetic modification to increase activity relative to its wild-type cell of at least two enzymes selected from E₁, E₂, and E₃, wherein:
E₁ is a lysine decarboxylase (EC: 4.1.1.18) that converts lysine to cadaverine;
E₂ is a cadaverine N5-monooxygenase (EC 1.14.13.-) that converts cadaverine to N5-hydroxy-cadaverine; and
E₃ is a N5-Aminopentyl-N-(hydroxy)-succinamic acid synthase (EC: 2.3.-.-) that converts N5-hydroxy-cadaverine and succinyl-coenzyme A to N5-aminopentyl-N-(hydroxy)-succinamic acid or N-hydroxy-N-succinyl putrescine.

3. The cell according to claim 2, wherein the cell comprises a genetic modification to increase the activity relative to its wild-type cell of the enzymes E₂ and E₃.

4. The cell according to claim 2, wherein the cell comprises a genetic modification to increase the activity relative to its wild-type cell of the three enzymes E₁, E₂, and E₃.

5. The cell according to claim 2, wherein the genetic modification is
a) at least one promoter which is operably linked to gene(s) encoding the enzymes E₁, E₂, and/or E₃ introduced in a suitable chromosome of the cell, or
b) at least one expression vector to increase the copy number of gene(s) encoding the enzymes E₁, E₂, and/or E₃ in the cell, or
c) combination of (a) and (b)
to increase the expression of the enzymes E₁, E₂, and/or E₃.

6. The cell according to claim 1, wherein the further genetic modification in the cell is
(i) an increase in activity relative to the wild-type cell of at least one of the following enzymes:
pyruvate carboxylase (EC 6.4.1.1) (E₆),
aspartate amino transferase (EC 2.6.1.1) (E₇),
aspartate kinase, particularly feedback resistant aspartate kinase (EC 2.7.2.4) (E₈),
aspartate semialdehyde dehydrogenase (EC 1.2.1.11) (E₉),
dihydrodipicolinate synthase (EC 4.3.3.7) (E₁₀),
dihydrodipicolinate reductase (EC 30 1.17.1.8) (E₁₁),
diaminopimelate dehydrogenase (EC 1.4.1.16) (E₁₂),
diaminopimelate epimerase (EC 5.1.1.7) (E₁₃),
diaminopimelate decarboxylase (EC 4.1.1.20) (E₁₄)
N-succinyl-aminoketopimelate aminotranferase (EC 2.6.1.17) (E₁₇),
2,3,4,5-tetrahydropyridine-2,6-dicarboxylate N-succinyltransferase (EC 2.3.1.117) (E₁₈), and/or
succinyl-diaminopimelate desuccinylase (EC 3.5.1.18) (E₁₉);
and/or
(ii) a decrease in activity relative to the wild-type cell of at least one of the following enzymes:
phosphoenolpyruvate carboxykinase (EC 4.1.1.32) (E₁₅), and/or
homoserine dehydrogenase (EC 1.1.1.3) (E₁₆).

7. The cell according to claim 1, wherein the further genetic modification is:
a) at least one promoter which is operably linked to a gene encoding any one of the enzymes E₆-E₁₄, and E₁₇-E₁₉ in the suitable chromosome of the cell, or
b) at least one expression vector to increase the copy number of gene(s) encoding any one of the enzymes E₆-E₁₄, and E₁₇-E₁₉ in the cell, or
c) combination of (a) and (b)
to increase the activity of any one of the enzymes E₆-E₁₄, and E₁₇-E₁₉ in the cell and/or
d) a foreign DNA in the gene encoding at least one of enzymes E₁₅ and E₁₆;
e) deletion of at least one part of the gene encoding at least one of enzymes E₁₅ and E₁₆;
f) at least one point mutation, RNA interference (siRNA), antisense RNA in the gene and/or regulatory sequences of the gene encoding at least one of enzymes E₁₅ and E₁₆; or
g) combinations of (d), (e) and (f)
to decrease the activity of at least one of the enzymes E₁₅ and E₁₆ in the cell.

8. The cell according to claim 2, wherein
E₁ comprises at least 70% sequence identity relative to SEQ ID NO:15 (E₁ₐ), SEQ ID NO:25 (E_{1b}) or SEQ ID NO:50 (E_{1c});
E₂ comprises at least 70% sequence identity relative to SEQ ID NO:4 (E₂ₐ), SEQ ID NO:16 (E_{2b}), SEQ ID NO:26 (E_{2c}), SEQ ID NO:33 (E_{2d}), SEQ ID NO:39 (E₂ₑ) or SEQ ID NO:45 (E_{2f}); and
E₃ comprises at least 70% sequence identity relative to SEQ ID NO:5 (E₃ₐ), SEQ ID NO:17 (E_{3b}), SEQ ID NO:27 (E_{3c}), N-terminal domain of SEQ ID NO:46 (E_{3d}), or SEQ ID NO:40 (E₃ₑ) or SEQ ID NO:34 (E_{3f}).

9. The cell according to claim 1, wherein the cell is selected from the group consisting of Aspergillus sp., Corynebacterium sp., Brevibacterium sp., Bacillus sp., Acinetobacter sp., Alcaligenes sp., Lactobacillus sp., Paracoccus sp., Lactococcus sp., Candida sp., Pichia sp., Hansenula sp., Kluyveromyces sp., Saccharomyces sp., Escherichia sp., Zymomonas sp., Yarrowia sp., Methylobacterium sp., Ralstonia sp., Pseudomonas sp., Rhodospirillum sp., Rhodobacter sp., Burkholderia sp., Clostridium sp., and Cupriavidus sp.

10. The cell according to claim 1, wherein the cell comprises a further genetic modification to increase expression relative to a wild-type cell of at least one E₄ enzyme selected from the group consisting of:
desferrioxamine synthetase (EC 6.3.-.-) (E₄ᵢ) capable of converting N5-aminopentyl-N-(hydroxyl)-succinamic acid to desferrioxamine B or H;
desferrioxamine synthetase (EC 6.3.-.-) (E₄ᵢᵢ) capable of converting N5-aminopentyl-N-(hydroxyl)-succinamic acid to at least one desferrioxamine;
bisucaberin synthetase (EC 6.3.-.-) (E₄ᵢᵢᵢ) capable of converting N5-aminopentyl-N-(hydroxyl)-succinamic acid to bisucaberin B; and
bisucaberin synthetase (EC 6.3.-.-) (E₄ᵢᵥ) capable of converting N5-aminopentyl-N-(hydroxyl)-succinamic acid to bisucaberin.

11. The cell according to claim 1, wherein the cell comprises a further genetic modification to increase expression relative to a wild-type cell of at least one E₄ enzyme selected from the group consisting of:
desferrioxamine synthetase (EC 6.3.-.-) (E₄ᵢ) capable of converting N5-aminopentyl-N-(hydroxyl)-succinamic acid to desferrioxamine B or H; and
bisucaberin synthetase (EC 6.3.-.-) (E₄ᵢᵢᵢ) capable of converting N5-aminopentyl-N-(hydroxyl)-succinamic acid to bisucaberin B
to produce at least one compound of Formula II: 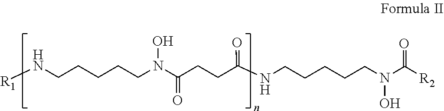
n=1-3
R₁═H or COCH₃ or CH₂CH₂COX with X═OH or O—
R₂═CH₃ or CH₂CH₂COX with X═OH or O—.

12. A method of producing at least one compound of Formula I 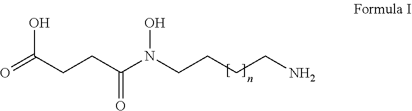
where n is 2
from at least one simple carbon source, the method comprising:
(a) contacting the cell according to claim 1 with at least one simple carbon source, wherein the simple carbon source is selected from the group consisting of glucose, sucrose, xylose, arabinose, mannose and glycerol.

13. The method according to claim 12, wherein the activity of the enzyme is increased in the cell by a method selected from the group consisting of
a) introducing at least one promoter which is operably linked to the gene encoding the enzymes into the chromosome of the cell,
b) increasing copy number of the gene encoding the enzyme by introducing at least one expression vector into the cell, and
c) combinations thereof.

14. Use of the cell according to claim 1 for producing at least one compound of Formula I from at least one simple carbon source 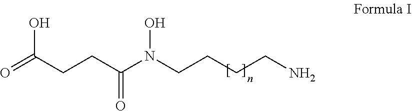
where n is 2 and wherein the simple carbon source is selected from the group consisting of glucose, sucrose, xylose, arabinose, mannose and glycerol.
